(19) United States
(12) Patent Application Publication
(10) Pub. No.: US 2019/0256838 A1
(43) Pub. Date: Aug. 22, 2019

(54) METHODS OF PRODUCING ASTAXANTHIN OR PRECURSORS THEREOF

(71) Applicant: DEINOVE, GRABELS (FR)

(72) Inventors: Sandra CASTANG, TEYRAN (FR); Philippe BAUCHART, GRABELS (FR); Alice KINDERMANS, GARRIGUES (FR); Nicolas CHABOT, TOULOUSE (FR); Jean-Paul LEONETTI, CASTELNAU LE LEZ (FR)

(21) Appl. No.: 16/307,983

(22) PCT Filed: Jun. 9, 2017
(86) PCT No.: PCT/EP2017/064134

§ 371 Date: Dec. 7, 2018

(30) Foreign Application Priority Data
Jun. 9, 2016 (EP) 16173768.9

(51) Int. Cl. C12N 9/90; C12N 9/04; C12P 23/00

(52) U.S. Cl. CPC C12N 9/90; C12Y 505/01019; C12P 23/00; C12N 9/0006; C12Y 114/13129

(57) ABSTRACT

The present invention relates to a recombinant Deinococcus bacterium comprising a heterologous biosynthetic pathway converting lycopene to astaxanthin or a precursor thereof, and its use for producing astaxanthin or a precursor thereof. Shown is the production of carotene, zeaxanthin, canthaxanthin and astaxanthin using recombinant Deinococcus expressing genes encoding for lycopene cylase (EC 5.5.1.19), beta-carotene hydroxylase (EC1.14.13.129) and beta-carotene ketolase, namely carotenoid-4,4-beta-ionone ring oxygenase (EC 1.14.11.B16).

### FIELD OF THE INVENTION

The present invention relates to the field of microbiology and in particular to the field of biosynthetic pathway engineering. More specifically, the present invention relates to the field of production of astaxanthin or a precursor thereof, using genetically modified bacteria.

### BACKGROUND OF THE INVENTION

Carotenoids are a class of natural pigments that are synthesized by all photosynthetic organisms and in some heterotrophic growing bacteria and fungi. Because animals are unable to synthetize de novo these molecules, carotenoids have been widely used commercially as food supplements, animal feed additives or nutraceuticals. They have also found various applications as colorants or for cosmetic and pharmaceutical purposes.

One of these molecules, namely astaxanthin, was found to exhibit various biological activities such as antioxidant, anti-lipid peroxidation, anti-inflammation, anti-diabetic, anti-aging or anti-cancer activities, as well as beneficial effects to prevent cardiovascular diseases, to preserve immune-system defences from free radicals damages or as neuroprotective agent.

Astaxanthin may be derived from natural sources or may be produced through chemical synthesis. Natural sources of astaxanthin include algae, yeast, salmon, trout, krill, shrimp, crayfish, and microorganism sources. On a commercial basis, astaxanthin is derived mainly from Haematococcus pluvialis microalgae and Xanthophyllomyces dendrorhous (also called Phaffia rhodozyma) red yeast but also from Paracoccus carotinifaciens bacterium and through chemical synthesis. Metabolic engineering of microorganisms offers an alternative and promising approach for the economical production of large amount of carotenoids. In this respect, many of the genes involved in carotenoid biosynthesis have been heterologously expressed in a variety of host cells such as Escherichia coli (Ruther et al., Appl Microbiol Biotechnol. 1997, 48(2):162-7), Candida utilis (Miura et al., Appl Environ Microbiol. 1998, 64(4):1226-9), or photosynthetic bacteria such as Rhodobacter sphaeroides (Hunter et al., J bacteriol. 1994, 176: 3692-3697) or Rhodovulum sulfidophilum (Mukoyama et al., FEMS Microbiol Lett. 2006, 265: 69-75).

Although engineering microorganisms to produce carotenoids such as astaxanthin has shown great promise, high yields of production require not only optimization of the isoprenoid precursor pool, but also the correct expression and cooperation of enzymes involved in the conversion of lycopene or β-carotene to the carotenoid of interest, such as astaxanthin, in order to maximize the production of said carotenoid and minimize the accumulation of undesired intermediates.

To date, genetically modified bacteria do not allow a cost-competitive industrial production process, in particular from cheap carbon sources such as cellulosic biomass. Consequently, there is still a strong need for a much improved process achieving industrially relevant productivity of carotenoids, and in particular astaxanthin, from renewable carbon sources.

### SUMMARY OF THE INVENTION

Based on their solid knowledge of Deinococcus metabolism and genetics, the inventors demonstrated that Deinococcus bacteria can be genetically modified to produce substantial amounts of astaxanthin or valuable precursors such as canthaxanthin or zeaxanthin. They further demonstrated that, using the recombinant bacterium of the invention, the production can be carried out at a temperature greater than 40° C., in particular between 42° C. and 48° C., thereby reducing the risk of microbial contamination and decreasing the costs of subsequent steps.

Accordingly, in a first aspect, the present invention relates to a recombinant Deinococcus bacterium comprising a heterologous nucleic acid sequence encoding a polypeptide exhibiting lycopene cyclase activity. This recombinant bacterium may further comprise a heterologous nucleic acid sequence encoding a polypeptide exhibiting beta-carotene hydroxylase activity and/or a nucleic acid sequence encoding a polypeptide exhibiting beta-carotene ketolase activity.

The polypeptide exhibiting lycopene cyclase activity may be selected from the group consisting of lycopene cyclases from organisms belonging to the genera Pantoea, Marchantia, Haematococcus, Deinococcus, Porphyrobacter, Franconibacter, Phormidium, Siccibacter, Erythrobacter, Rhizobium and Parvularcula, preferably from Pantoea, Deinococcus, Porphyrobacter, Franconibacter, Phormidium, Siccibacter, Erythrobacter, Rhizobium and Parvularcula. The polypeptide exhibiting lycopene cyclase activity may also be selected from the group consisting of lycopene cyclases from organisms belonging to the genera Pantoea, Marchantia, Haematococcus and Deinococcus.

In particular, the polypeptide exhibiting lycopene cyclase activity may be selected from the group consisting of lycopene cyclases from Pantoea ananatis, Marchantia polymorpha, Haematococcus pluvialis, Deinococcus deserti, Pantoea agglomerans, Porphyrobacter cryptus, Franconibacter pulveris, Phormidium tenue, Siccibacter colletis, Erythrobacter vulgaris, Rhizobium sp. Leaf321 and Parvularcula oceani, preferably from Pantoea ananatis, Deinococcus deserti, Pantoea agglomerans, Porphyrobacter cryptus, Franconibacter pulveris, Phormidium tenue, Siccibacter colletis, Erythrobacter vulgaris, Rhizobium sp. Leaf321 and Parvularcula oceani. Alternatively, the polypeptide exhibiting lycopene cyclase activity may be selected from the group consisting of lycopene cyclases from Pantoea ananatis, Marchantia polymorpha, Haematococcus pluvialis, Deinococcus deserti, Pantoea agglomerans.

In particular, the polypeptide exhibiting lycopene cyclase activity is selected from the group consisting of lycopene cyclases of SEQ ID NO: 1, 3, 5, 7, 9, 75, 77, 79, 81, 83, 85 and 87, and polypeptides exhibiting lycopene cyclase activity and having at least 60%, preferably at least 80% or 90%, identity to SEQ ID NO: 1, 3, 5, 7, 9, 75, 77, 79, 81, 83, 85 and 87. Preferably, the polypeptide exhibiting lycopene cyclase activity is selected from the group consisting of lycopene cyclases of SEQ ID NO: 1, 7, 9, 75, 77, 79, 81, 83, 85 and 87, and polypeptides exhibiting lycopene cyclase activity and having at least 60%, preferably at least 80% or 90%, identity to SEQ ID NO: 1, 7, 9, 75, 77, 79, 81, 83, 85 or 87. Alternatively, the polypeptide exhibiting lycopene cyclase activity is selected from the group consisting of lycopene cyclases of SEQ ID NO: 1, 3, 5, 7 and 9, and polypeptides exhibiting lycopene cyclase activity and having at least 60%, preferably at least 80% or 90%, identity to SEQ ID NO: 1, 3, 5, 7 or 9. More preferably, the polypeptide exhibiting lycopene cyclase activity is selected from the group consisting of lycopene cyclases of SEQ ID NO: 1 and 7, and polypeptides exhibiting lycopene cyclase activity and having at least 60%, preferably at least 80% or 90%, identity to SEQ ID NO: 1 or 7.

The polypeptide exhibiting beta-carotene hydroxylase activity may be selected from the group consisting of beta-carotene hydroxylases from organisms belonging to the genera Pantoea, Marchantia, Haematococcus, Tagetes, Brevundimonas, Paracoccus, Franconibacter, Siccibacter and Cronobacter, preferably from Pantoea, Franconibacter, Siccibacter and Cronobacter. Alternatively, the polypeptide exhibiting beta-carotene hydroxylase activity may be selected from the group consisting of beta-carotene hydroxylases from organisms belonging to the genera Pantoea, Marchantia, Haematococcus, Tagetes, Brevundimonas and Paracoccus.

Preferably, the polypeptide exhibiting beta-carotene hydroxylase activity may be selected from the group consisting of beta-carotene hydroxylases from Pantoea agglomerans, Pantoea stewartii, Pantoea ananatis, Marchantia polymorpha, Haematococcus pluvialis, Tagetes erecta, Brevundimonas sp. SD212, Paracoccus sp. N81106, Franconibacter pulveris, Siccibacter colletis and Cronobacter malonaticus, preferably from Pantoea agglomerans, Franconibacter pulveris, Siccibacter colletis and Cronobacter malonaticus. Alternatively, the polypeptide exhibiting beta-carotene hydroxylase activity may be selected from the group consisting of beta-carotene hydroxylases from Pantoea agglomerans, Pantoea stewartii, Pantoea ananatis, Marchantia polymorpha, Haematococcus pluvialis, Tagetes erecta, Brevundimonas sp. SD212 and Paracoccus sp. N81106. Preferably, the polypeptide exhibiting beta-carotene hydroxylase activity is selected from the group consisting of beta-carotene hydroxylases of SEQ ID NO: 11, 13, 15, 17, 19, 21, 23, 25, 89, 91 and 93, and polypeptides exhibiting beta-carotene hydroxylase activity and having at least 60%, preferably at least 80% or 90%, identity to SEQ ID NO: 11, 13, 15, 17, 19, 21, 23, 25, 89, 91 or 93. Alternatively, the polypeptide exhibiting beta-carotene hydroxylase activity is selected from the group consisting of beta-carotene hydroxylases of SEQ ID NO: 11, 13, 15, 17, 19, 21, 23 and 25, and polypeptides exhibiting beta-carotene hydroxylase activity and having at least 60%, preferably at least 80% or 90%, identity to SEQ ID NO: 11, 13, 15, 17, 19, 21, 23 or 25.

More preferably, the polypeptide exhibiting beta-carotene hydroxylase activity is selected from the group consisting of beta-carotene hydroxylases of SEQ ID NO: 13, 89, 91 and 93, and polypeptides exhibiting beta-carotene hydroxylase activity and having at least 60%, preferably at least 80% or 90%, identity to SEQ ID NO: 13, 89, 91 or 93. In particular, the polypeptide exhibiting beta-carotene hydroxylase activity is selected from the group consisting of beta-carotene hydroxylase of SEQ ID NO: 13, and polypeptides exhibiting beta-carotene hydroxylase activity and having at least 60%, preferably at least 80% or 90%, identity to SEQ ID NO: 13.

The polypeptide exhibiting beta-carotene ketolase activity may be selected from the group consisting of beta-carotene ketolases from organisms belonging to the genera Deinococcus, Brevundimonas, Paracoccus, Haematococcus, Erythrobacter, Sphingomonas, Bradyrhizobium, Nostoc and Chlamydomonas, preferably from Deinococcus and Paracoccus, more preferably from Deinococcus.

Preferably, the polypeptide exhibiting beta-carotene ketolase activity may be selected from the group consisting of beta-carotene ketolases from Deinococcus geothermalis, Deinococcus murrayi, Deinococcus maricopensis, Brevundimonas sp. SD212, Paracoccus sp. PC1, Paracoccus sp. N81106, Paracoccus haeundaensis, Paracoccus sanguinis (Paracoccus sp. 39524), Paracoccus sphaerophysae, Haematococcus pluvialis, Erythrobacter litoralis, Sphingomonas sp. PB304, Sphingomonas sp. SRS2, Bradyrhizobium sp. ORS 278, Nostoc sp. PCC 7120 and Chlamydomonas reinhardtii, preferably from Deinococcus geothermalis, Brevundimonas sp. SD212, Paracoccus sp. PC1, Paracoccus sp. N81106, Paracoccus haeundaensis, Paracoccus sanguinis (Paracoccus sp. 39524), Paracoccus sphaerophysae, Haematococcus pluvialis, Erythrobacter litoralis, Sphingomonas sp. PB304, Sphingomonas sp. SRS2, Bradyrhizobium sp. ORS 278, Nostoc sp. PCC 7120 and Chlamydomonas reinhardtii, more preferably from Deinococcus geothermalis and Paracoccus sp. N81106, and even more preferably from Deinococcus geothermalis.

Preferably, the polypeptide exhibiting beta-carotene ketolase activity is selected from the group consisting of beta-carotene ketolase of SEQ ID NO: 27, 29, 31, 33, 35, 37, 39, 41, 43, 45, 47, 67, 69, 71, 73, 95 and 97, and polypeptides exhibiting beta-carotene ketolase activity and having at least 60%, preferably at least 80% or 90%, identity to SEQ ID NO: 27, 29, 31, 33, 35, 37, 39, 41, 43, 45, 47, 67, 69, 71, 73, 95 or 97.

More preferably, the polypeptide exhibiting beta-carotene ketolase activity is selected from the group consisting of beta-carotene ketolase of SEQ ID NO: 27, 29, 31, 33, 35, 37, 39, 41, 43, 45, 47, 67, 69, 71 and 73, and polypeptides exhibiting beta-carotene ketolase activity and having at least 60%, preferably at least 80% or 90%, identity to SEQ ID NO: 27, 29, 31, 33, 35, 37, 39, 41, 43, 45, 47, 67, 69, 71 and 73, even more preferably selected from the group consisting of beta-carotene ketolase of SEQ ID NO: 27, 29, 31 and 47, and polypeptides exhibiting beta-carotene ketolase activity and having at least 60%, preferably at least 80% or 90%, identity to SEQ ID NO: 27, 29, 31 or 47.

In particular, the polypeptide exhibiting beta-carotene ketolase activity is selected from the group consisting of beta-carotene ketolases of SEQ ID NO: 31, 47, 95 and 97, and polypeptides exhibiting beta-carotene ketolase activity and having at least 60%, preferably at least 80% or 90%, identity to SEQ ID NO: 31, 47, 95 or 97, preferably from the group consisting of beta-carotene ketolases of SEQ ID NO: 31 and 47, and polypeptides exhibiting beta-carotene ketolase activity and having at least 60%, preferably at least 80% or 90%, identity to SEQ ID NO: 31 or 47, more preferably from the group consisting of beta-carotene ketolase of SEQ ID NO: 47, and polypeptides exhibiting beta-carotene ketolase activity and having at least 60%, preferably at least 80% or 90%, identity to SEQ ID NO: 47.

In some preferred embodiments, the polypeptide exhibiting beta-carotene ketolase activity is a beta-carotene ketolase from a thermophilic Deinococcus bacterium, preferably from Deinococcus geothermalis, Deinococcus murrayi or Deinococcus maricopensis. In particular, the polypeptide exhibiting beta-carotene ketolase activity may be selected from the group consisting of beta-carotene ketolases of SEQ ID NO: 47, 95 and 97, and polypeptides exhibiting beta-carotene ketolase activity and having at least 60%, preferably at least 80% or 90%, identity to SEQ ID NO: 47, 95 or 97.

In the recombinant bacterium of the invention, the endogenous gene encoding LmbE like protein, the gene encoding carotenoid 3′,4′-desaturase (CrtD), the gene encoding glucosyltransferase (CruC), the gene encoding acyltransferase (CruD), the gene encoding C-1′,2′ hydratase (CruF) and/or the gene encoding endogenous CrtO-type ketolase may be inactivated.

The recombinant bacterium of the invention may further exhibit increased FPP synthase, DXP synthase, IPP isomerase, phytoene synthase and/or phytoene desaturase activities by comparison to the wild-type bacterium, preferably increased FPP synthase, DXP synthase, IPP isomerase, phytoene synthase and phytoene desaturase activities by comparison to the wild-type bacterium.

Preferably, the recombinant bacterium of the invention is a thermophilic Deinococcus, more preferably Deinococcus geothermalis, Deinococcus murrayi or Deinococcus maricopensis, even more preferably Deinococcus geothermalis.

Preferably, the recombinant bacterium of the invention is able to produce astaxanthin, or a precursor thereof, when cultured at a temperature greater than 40° C., in particular between 40° C. and 48° C., preferably between 42° C. and 48° C. More preferably, said recombinant is able to produce beta-carotene, zeaxanthin and/or canthaxantin when cultured at a temperature greater than 40° C., preferably between 40° C. and 48° C., more preferably between 42° C. and 48° C.

In a second aspect, the present invention also relates to a method of producing a compound selected from astaxanthin and a precursor thereof, comprising culturing a recombinant Deinococcus bacterium according to the invention, under conditions suitable to produce said compound, and optionally recovering said compound. In particular, the compound may be an astaxanthin precursor may be selected from the group consisting of β-carotene, echinenone, canthaxantin, adonirubin, 3′hydroxyechinenone, 3-hydroxyechinenone, β-cryptoxanthin, zeaxanthin and adonixanthin, preferably selected from the group consisting of β-carotene, zeaxanthin and canthaxantin.

Preferably, in the method of the invention, the recombinant Deinococcus bacterium of the invention is cultured at a temperature between 37° C. and 52° C., preferably between 40° C. and 50° C., more preferably between 40° C. and 48° C., and even more preferably between 42° C. and 48° C.

The present invention also relates to the use of a recombinant Deinococcus bacterium according to the invention to produce a compound selected from astaxanthin and a precursor thereof. Preferably, said compound is selected from the group consisting of β-carotene, echinenone, canthaxantin, adonirubin, 3′hydroxyechinenone, 3-hydroxyechinenone, β-cryptoxanthin, zeaxanthin, adonixanthin and astaxanthin, or a mixture thereof. More preferably, said compound is selected from the group consisting of β-carotene, echinenone, canthaxantin, adonirubin, 3′hydroxyechinenone, 3-hydroxyechinenone, β-cryptoxanthin, zeaxanthin and adonixanthin, and a mixture thereof. Even more preferably, said compound is selected from the group consisting of β-carotene, canthaxantin and zeaxanthin, and a mixture thereof.

The present invention also relates to a cell extract of a recombinant Deinococcus bacterium of the invention and its use to produce astaxanthin or a precursor thereof, or a mixture thereof.

The present invention also relates to a composition comprising a recombinant Deinococcus bacterium of the invention or an extract thereof and the use of said composition to produce astaxanthin or a precursor thereof, or a mixture thereof.

The present invention further relates to a reactor comprising a recombinant Deinococcus bacterium of the invention, or a cell extract thereof, and its use to produce astaxanthin, or a precursor thereof, or a mixture thereof.

The invention also relates to astaxanthin or a precursor thereof, preferably isolated or purified astaxanthin or precursor, obtained by a method of the invention.

### BRIEF DESCRIPTION OF THE DRAWINGS

FIG. 1: HPLC spectra of the wild-type D. geothermalis strain (A) and the recombinant D. geothermalis (strain A) having a disrupted carotenoid pathway and overexpressing FPPS (B).

FIG. 2: β-carotene production of a D. geothermalis strain expressing CrtY from Pantoea ananatis (strain B) and a D. geothermalis strain expressing CrtY from Deinococcus deserti (strain C). β-carotene production is expressed in mg/g of cell dry weight (CDW) or mg/g of glucose consumed.

FIG. 3: HPLC spectrum of a D. geothermalis strain expressing CrtY from Pantoea ananatis and CrtZ from Pantoea agglomerans (strain D).

FIG. 4: HPLC spectrum of a D. geothermalis strain expressing CrtY from Pantoea ananatis and CrtW from Brevundimonas sp. SD212 (strain E).

FIG. 5: HPLC spectrum of a D. geothermalis strain expressing CrtY from Pantoea ananatis and CrtW from Paracoccus sp. N81106 (strain F).

FIG. 6: HPLC spectrum of a D. geothermalis strain expressing CrtY from Pantoea ananatis and CrtO from D. geothermalis (strain G).

FIG. 7: HPLC spectrum of a D. geothermalis strain expressing CrtY from Pantoea ananatis, CrtZ from Pantoea agglomerans and CrtO from D. geothermalis (strain H).

FIG. 8: HPLC spectrum of a D. geothermalis strain expressing CrtY from Pantoea ananatis, CrtZ from Pantoea agglomerans and CrtW from Paracoccus sp. N81106 (strain I).

### DETAILED DESCRIPTION OF THE INVENTION

Deinococcus bacteria are non-pathogen bacteria that were firstly isolated in 1956 by Anderson and collaborators. These extremophile organisms have been proposed for use in industrial processes or reactions using biomass (see e.g., WO2009/063079; WO2010/094665 or WO2010/081899).

Based on their solid knowledge of Deinococcus metabolism and genetics, the inventors found that Deinococcus bacteria can be genetically modified to produce substantial amounts of astaxanthin or precursors thereof under conditions compatible with large scale production and using industrial substrates.

### Definitions

In the context of the invention, the term “Deinococcus” includes wild type or natural variant strains of Deinococcus, e.g., strains obtained through accelerated evolution, mutagenesis, by DNA-shuffling technologies, or recombinant strains obtained by insertion of eukaryotic, prokaryotic and/or synthetic nucleic acid(s). Deinococcus bacteria can designate any bacterium of the genus Deinococcus, such as without limitation. D. actinosclerus, D. aerius, D. aerolatus, D. aerophilus, D. aetherius, D. alpinitundrae, D. altitudinis, D. antarcticus, D. apachensis, D. aquaticus, D. aquaticus, D. aquatilis, D. aquiradiocola, D. caeni, D. carri, D. cellulosilyticus, D. citri, D. claudionis, D. daejeonensis, D. depolymerans, D. desertii, D. enclensis, D. ficus, D. frigens, D. geothermalis, D. gobiensis, D. grandis, D. guangriensis, D. guilhemensis, D. hohokamensis, D. hopiensis, D. humi, D. indicus, D. maricopensis, D. marmoris, D. metalli, D. metallilatus, D. misasensis, D. murrayi, D. navajonensis, D. papagonensis, D. peraridilitoris, D. phoenicis, D. pimensis, D. piscis, D. proteolyticus, D. puniceus, D. radiodurans, D. radiomollis, D. radiophilus, D. radiopugnans, D. radioresistens, D. radiotolerans, D. reticulitermitis, D. roseus, D. sahariens, D. saxicola, D. soli, D. sonorensis, D. swuensis, D. wulumuqiensis, D. xinjiangensis, D. xibeiensis and D. yavapaiensis bacterium, or any combinations thereof. Preferably, the term “Deinococcus” refers to D. geothermalis, D. murrayi, D. grandis, D. aquaticus, D. indicus, D. cellulosilyticus, D. depolymerans. More preferably, the term “Deinococcus” refers to D. geothermalis.

In some preferred embodiments, the term “Deinococcus” refers to a thermophilic Deinococcus, i.e. a Deinococcus which is able to grow at a temperature of more than 40° C., preferably between 40° C. and 50° C., more preferably between 42° C. and 48° C., and even more preferably at about 45° C. In particular, the thermophilic Deinococcus may be selected from the group consisting of D. murrayi, D. maricopensis and D. geothermalis. Preferably, the thermophilic Deinococcus is D. geothermalis.

As used herein, the term “Paracoccus” refers to α-proteobacteria (NCBI Taxonomy ID: 265) and not scale insects.

The term “recombinant bacterium” or “genetically modified bacterium” designates a bacterium that is not found in nature and which contains a modified genome as a result of either a deletion, insertion or modification of one or several genetic elements.

A “recombinant nucleic acid” designates a nucleic acid which has been engineered and is not found as such in wild type bacteria. In some particular embodiments, this term may refer to a gene operably linked to a promoter that is different from its naturally occurring promoter.

The term “gene” designates any nucleic acid encoding a protein. The term gene encompasses DNA, such as cDNA or gDNA, as well as RNA. The gene may be first prepared by e.g., recombinant, enzymatic and/or chemical techniques, and subsequently replicated in a host cell or an in vitro system. The gene typically comprises an open reading frame encoding a desired protein. The gene may contain additional sequences such as a transcription terminator or a signal peptide.

The term “operably linked” means a configuration in which a control sequence is placed at an appropriate position relative to a coding sequence, in such a way that the control sequence directs expression of the coding sequence.

The term “control sequences” means nucleic acid sequences necessary for expression of a gene. Control sequences may be native or heterologous. Well-known control sequences and currently used by the person skilled in the art will be preferred. Such control sequences include, but are not limited to, a leader, polyadenylation sequence, propeptide sequence, promoter, signal peptide sequence, and transcription terminator. Preferably, the control sequences include a promoter and a transcription terminator.

The term “expression cassette” denotes a nucleic acid construct comprising a coding region, i.e. a gene, and a regulatory region, i.e. comprising one or more control sequences, operably linked. Preferably, the control sequences are suitable for Deinococcus host cells.

As used herein, the term “expression vector” means a DNA or RNA molecule that comprises an expression cassette. Preferably, the expression vector is a linear or circular double stranded DNA molecule.

As used herein, the term “native” or “endogenous”, with respect to a bacterium, refers to a genetic element or a protein naturally present in said bacterium. The term “heterologous”, with respect to a bacterium, refers to a genetic element or a protein that is not naturally present in said bacterium.

The terms “overexpression” and “increase expression” as used herein, are used interchangeably and mean that the expression of a gene or an enzyme is increased compared to a non modified bacterium, e.g. the wild-type bacterium or the corresponding bacterium that has not been genetically modified in order to produce astaxanthine or a precursor thereof. Increased expression of an enzyme is usually obtained by increasing expression of the gene encoding said enzyme. In embodiments wherein the gene or the enzyme is not naturally present in the bacterium of the invention, i.e. heterologous gene or enzyme, the terms “overexpression” and “expression” may be used interchangeably. To increase the expression of a gene, the skilled person can used any known techniques such as increasing the copy number of the gene in the bacterium, using a promoter inducing a high level of expression of the gene, i.e. a strong promoter, using elements stabilizing the corresponding messenger RNA or modifying Ribosome Binding Site (RBS) sequences and sequences surrounding them. In particular, the overexpression may be obtained by increasing the copy number of the gene in the bacterium. One or several copies of the gene may be introduced into the genome by methods of recombination, known to the expert in the field, including gene replacement or multicopy insertion in IS sequences (see for example the international patent application WO 2015/092013). Preferably, an expression cassette comprising the gene, preferably placed under the control of a strong promoter, is integrated into the genome. Alternatively, the gene may be carried by an expression vector, preferably a plasmid, comprising an expression cassette with the gene of interest preferably placed under the control of a strong promoter. The expression vector may be present in the bacterium in one or several copies, depending on the nature of the origin of replication. The overexpression of the gene may also obtained by using a promoter inducing a high level of expression of the gene. For instance, the promoter of an endogenous gene may be replaced by a stronger promoter, i.e. a promoter inducing a higher level of expression. The promoters suitable to be used in the present invention are known by the skilled person and can be constitutive or inducible, and native or heterologous.

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5. In some embodiments, all the identity percentages mentioned in this application may be set to at least 80%, preferably to at least 90% identity, more preferably to at least 95% identity.

The terms “low stringency”, “medium stringency”, “medium/high stringency”, “high stringency” and “very high stringency” refer to conditions of hybridization. Suitable experimental conditions for determining hybridization between a nucleotide probe and a homologous DNA or RNA sequence involves presoaking of the filter containing the DNA fragments or RNA to hybridize in 5×SSC (Sodium chloride/Sodium citrate for 10 min, and prehybridization of the filter in a solution of 5×SSC, 5×Denhardt's solution, 0.5% SDS and 100 μg/ml of denatured sonicated salmon sperm DNA, followed by hybridization in the same solution containing a concentration of 10 ng/ml of a random-primed ³²P-dCTP-labeled (specific activity >1×10⁹ cpm/μg) probe for 12 hours at ca. 45° C. (Feinberg and Vogelstein, 1983). For various stringency conditions the filter is then washed twice for 30 minutes in 2×SSC, 0.5% SDS and at least 55° C. (low stringency), more preferably at least 60° C. (medium stringency), still more preferably at least 65° C. (medium/high stringency), even more preferably at least 70° C. (high stringency), and even more preferably at least 75° C. (very high stringency).

As used in this specification, the term “about” refers to a range of values ±10% of the specified value. For example, “about 20” includes ±10% of 20, or from 18 to 22. Preferably, the term “about” refers to a range of values ±5% of the specified value.

As used herein, the term “CrtY” or “lycopene cyclase” refers to a lycopene cyclase enzyme (EC 5.5.1.19) encoded by a crtY gene which converts lycopene to β-carotene.

The term “CrtZ” or “β-carotene hydroxylase” refers to a β-carotene hydroxylase enzyme (EC 1.14.13.129) encoded by a crtZ gene which catalyzes a hydroxylation reaction from β-carotene to zeaxanthin or from canthaxanthin to astaxanthin.

The term “beta-carotene ketolase” refers to a carotenoid ketolase that introduces keto groups to the β-ionone ring of the cyclic carotenoids such as β-carotene. This term may be used to refer to CrtW-type ketolases or to CrtO-type ketolases. The term “CrtW” refers to a CrtW-type ketolase or carotenoid 4,4′-beta-ionone ring oxygenase enzyme (EC 1.14.11.B16) which converts β-carotene to canthaxantin or zeaxanthine to astaxanthine. The term “CrtO” refers to a CrtO-type ketolase or beta-carotene monoketolase enzyme (EC 1.3.5.B4) which converts β-carotene to produce canthaxanthin or converts zeaxanthin to produce astaxanthin.

According to the organism, the nomenclature of the above identified enzymes and encoding genes may vary. However, for the sake of clarity, in the present specification, these terms are used independently from the origin of the enzymes or genes.

Heterologous Astaxanthin Biosynthetic Pathway

In a first aspect, the present invention relates to a recombinant Deinococcus bacterium comprising a heterologous nucleic acid sequence encoding a polypeptide exhibiting lycopene cyclase activity (CrtY), a heterologous nucleic acid sequence encoding a polypeptide exhibiting beta-carotene hydroxylase activity (CrtZ) and/or a nucleic acid sequence encoding a polypeptide exhibiting beta-carotene ketolase activity (CrtW/O). The invention indeed shows that such functional proteins may be expressed in Deinococcus bacteria in suitable amounts and without altering the bacterial viability or growth.

Accordingly, the present invention firstly relates to a recombinant Deinococcus bacterium comprising a heterologous nucleic acid sequence encoding a polypeptide exhibiting lycopene cyclase activity (CrtY). Preferably, said recombinant bacterium is able to produce beta-carotene when cultured at a temperature between 42° C. and 50° C., preferably between 42 and 48° C., more preferably at about 42° C., 45° C. or 48° C.

The polypeptide exhibiting lycopene cyclase activity may be any known lycopene cyclase, in particular selected from known bacterial, algal or plant lycopene cyclases, preferably from bacterial lycopene cyclases, more preferably from lycopene cyclases from Gram negative bacteria.

In particular, the polypeptide exhibiting lycopene cyclase activity may be selected from the group consisting of lycopene cyclases from organisms belonging to the genera Pantoea, Marchantia, Haematococcus, Deinococcus, Porphyrobacter, Franconibacter, Phormidium, Siccibacter, Erythrobacter, Rhizobium and Parvularcula, preferably from Pantoea, Deinococcus, Porphyrobacter, Franconibacter, Phormidium, Siccibacter, Erythrobacter, Rhizobium and Parvularcula. Alternatively, the polypeptide exhibiting lycopene cyclase activity may be selected from the group consisting of lycopene cyclases from organisms belonging to the genera Pantoea, Marchantia, Haematococcus and Deinococcus, preferably from organisms belonging to the genus Deinococcus or Pantoea.

Examples of species belonging to the Pantoea genus include, but are not limited to, Pantoea agglomerans, Pantoea ananatis, Pantoea stewartii, Pantoea citrea, Pantoea dispersa, Pantoea punctate, Pantoea terrea, Pantoea deleyi, Pantoea anthophila, Pantoea allii and Pantoea eucalypti. Preferably, the Pantoea bacterium is selected from the group consisting of Pantoea agglomerans, Pantoea ananatis and Pantoea stewartii, more preferably from Pantoea agglomerans and Pantoea ananatis, and even more preferably from Pantoea ananatis.

In an embodiment, the polypeptide exhibiting lycopene cyclase activity is selected from the group consisting of lycopene cyclases from Pantoea ananatis (NCBI Accession number: BAA14126; SEQ ID NO: 1), Marchantia polymorpha (NCBI Accession number: BAO27799; SEQ ID NO: 3), Haematococcus pluvialis (NCBI Accession number: AAO64977; SEQ ID NO: 5), Deinococcus deserti (NCBI Accession number: WP_012695258; SEQ ID NO: 7), Pantoea agglomerans (NCBI Accession number: AFZ89041; SEQ ID NO: 9), Porphyrobacter cryptus (NCBI Accession number: WP_027442625; SEQ ID NO: 75), Franconibacter pulveris (NCBI Accession number: WP_029590459.1; SEQ ID NO: 77), Phormidium tenue (NCBI Accession number: WP_073608653.1; SEQ ID NO: 79), Siccibacter colletis (NCBI Accession number: WP_031523884; SEQ ID NO: 81), Erythrobacter vulgaris (NCBI Accession number: WP_040965145.1; SEQ ID NO: 83), Rhizobium sp. Leaf321 (NCBI Accession number: WP_062583193.1; SEQ ID NO: 85) and Parvularcula oceani (NCBI Accession number: WP_031553302.1; SEQ ID NO: 87), preferably from Pantoea ananatis, Deinococcus deserti, Pantoea agglomerans, Porphyrobacter cryptus, Franconibacter pulveris, Phormidium tenue, Siccibacter colletis, Erythrobacter vulgaris, Rhizobium sp. Leaf321 and Parvularcula oceani. In an embodiment, the polypeptide exhibiting lycopene cyclase activity is selected from the group consisting of lycopene cyclases of SEQ ID NO: 1, 3, 5, 7 and 9. In a preferred embodiment, the polypeptide exhibiting lycopene cyclase activity is selected from the group consisting of lycopene cyclases of SEQ ID NO: 1, 7, 9, 75, 77, 79, 81, 83, 85 and 87. The polypeptide exhibiting lycopene cyclase activity may also be any polypeptide exhibiting lycopene cyclase activity and having at least 60%, preferably at least 65, 70, 75, 80, 85, 90, 95, 96, 97, 98 or 99%, identity to any lycopene cyclase listed above.

In a preferred embodiment, the polypeptide exhibiting lycopene cyclase activity is selected from the group consisting of

a) a polypeptide comprising, or consisting of, an amino acid sequence selected from the group consisting of SEQ ID NO: 1, 3, 5, 7, 9, 75, 77, 79, 81, 83, 85 and 87, preferably selected from the group consisting of SEQ ID NO: 1, 7, 9, 75, 77, 79, 81, 83, 85 and 87;

b) a polypeptide exhibiting lycopene cyclase activity and having an amino acid sequence having at least 60%, preferably at least 65, 70, 75, 80, 85, 90, 95, 96, 97, 98 or 99%, identity to SEQ ID NO: 1, 3, 5, 7, 9, 75, 77, 79, 81, 83, 85 and 87, preferably to SEQ ID NO: 1, 7, 9, 75, 77, 79, 81, 83, 85 and 87;

c) a polypeptide exhibiting lycopene cyclase activity encoded by a nucleotide sequence having at least 60%, preferably 65, 70, 75, 80, 85, 90, 95, 96, 97, 98 or 99%, identity to a nucleotide sequence selected from the group consisting of SEQ ID NO: 2, 4, 6, 8, 10, 76, 78, 80, 82, 84, 86 and 88, preferably selected from the group consisting of SEQ ID NO: 2, 8, 10, 76, 78, 80, 82, 84, 86 and 88;

d) a polypeptide exhibiting lycopene cyclase activity and which is encoded by a nucleic acid sequence which is capable of hybridizing under medium/high stringency, preferably high or very high, conditions with (i) a nucleotide sequence selected from the group consisting of SEQ ID NO: 2, 4, 6, 8, 10, 76, 78, 80, 82, 84, 86 and 88, preferably selected from the group consisting of SEQ ID NO: 2, 8, 10, 76, 78, 80, 82, 84, 86 and 88 (ii) its complementary strand, or (iii) a subsequence of (i) or (ii).

In a particular embodiment, the polypeptide exhibiting lycopene cyclase activity is selected from the group consisting of

a) a polypeptide comprising, or consisting of, an amino acid sequence selected from the group consisting of SEQ ID NO: 1, 3, 5, 7 and 9;

b) a polypeptide exhibiting lycopene cyclase activity and having an amino acid sequence having at least 60%, preferably at least 65, 70, 75, 80, 85, 90, 95, 96, 97, 98 or 99%, identity to SEQ ID NO: 1, 3, 5, 7 or 9;

c) a polypeptide exhibiting lycopene cyclase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 2, 4, 6, 8 and 10;

d) a polypeptide exhibiting lycopene cyclase activity and which is encoded by a nucleic acid sequence which is capable of hybridizing under medium/high stringency, preferably high or very high, conditions with (i) a nucleotide sequence selected from the group consisting of SEQ ID NO: 2, 4, 6, 8 and 10, (ii) its complementary strand, or (iii) a subsequence of (i) or (ii).

In a more particular embodiment, the polypeptide exhibiting lycopene cyclase activity is selected from the group consisting of polypeptides comprising, or consisting of, an amino acid sequence selected from the group consisting of SEQ ID NO: 1, 3, 5, 7, 9, 75, 77, 79, 81, 83, 85 and 87, preferably of SEQ ID NO: 1, 7, 9, 75, 77, 79, 81, 83, 85 and 87, and alternatively of SEQ ID NO: 1, 3, 5, 7 and 9, and polypeptides exhibiting lycopene cyclase activity and having at least 60%, preferably at least 65, 70, 75, 80, 85, 90, 95, 96, 97, 98 or 99%, identity to SEQ ID NO: 1, 3, 5, 7, 9, 75, 77, 79, 81, 83, 85 and 87, preferably to SEQ ID NO: 1, 7, 9, 75, 77, 79, 81, 83, 85 and 87, and alternatively to SEQ ID NO: 1, 3, 5, 7 or 9. Preferably, the polypeptide exhibiting lycopene cyclase activity is selected from the group consisting of polypeptides comprising, or consisting of, an amino acid sequence selected from the group consisting of SEQ ID NO: 1 and 7, and polypeptides exhibiting lycopene cyclase activity and having at least 60%, preferably at least 65, 70, 75, 80, 85, 90, 95, 96, 97, 98 or 99%, identity to SEQ ID NO: 1 or 7. More preferably, the polypeptide exhibiting lycopene cyclase activity is selected from the group consisting of a polypeptide comprising, or consisting of, an amino acid sequence of SEQ ID NO: 1, and polypeptides exhibiting lycopene cyclase activity and having at least 60%, preferably at least 65, 70, 75, 80, 85, 90, 95, 96, 97, 98 or 99%, identity to SEQ ID NO: 1. In a preferred embodiment, the polypeptide exhibiting lycopene cyclase activity comprises, or consists of, an amino acid sequence selected from the group consisting of SEQ ID NO: 1 and 7, preferably SEQ ID NO: 1.

In another preferred embodiment, the polypeptide exhibiting lycopene cyclase activity is selected from the group consisting of polypeptides comprising, or consisting of, an amino acid sequence selected from the group consisting of SEQ ID NO: 9, 75, 77 and 87, and polypeptides exhibiting lycopene cyclase activity and having at least 60%, preferably at least 65, 70, 75, 80, 85, 90, 95, 96, 97, 98 or 99%, identity to SEQ ID NO: 9, 75, 77 or 87. In particular, the polypeptide exhibiting lycopene cyclase activity is selected from the group consisting of a polypeptide comprising, or consisting of, an amino acid sequence of SEQ ID NO: 87, and polypeptides exhibiting lycopene cyclase activity and having at least 60%, preferably at least 65, 70, 75, 80, 85, 90, 95, 96, 97, 98 or 99%, identity to SEQ ID NO: 87.

The recombinant Deinococcus bacterium may further comprise a heterologous nucleic acid sequence encoding a polypeptide exhibiting beta-carotene hydroxylase activity (CrtZ). Preferably, said recombinant bacterium exhibits beta-carotene hydroxylase activity when cultured at a temperature between 42° C. and 50° C., preferably between 42 and 48° C., and more preferably at about 42° C., 45° C. or 48° C.

The polypeptide exhibiting beta-carotene hydroxylase activity may be any known beta-carotene hydroxylase, in particular selected from known bacterial, algal or plant beta-carotene hydroxylases, preferably from bacterial beta-carotene hydroxylases, more preferably from beta-carotene hydroxylases from Gram negative bacteria.

In particular, the polypeptide exhibiting beta-carotene hydroxylase activity may be selected from the group consisting of beta-carotene hydroxylases from organisms belonging to the genera Pantoea, Marchantia, Haematococcus, Tagetes, Brevundimonas, Paracoccus, Franconibacter, Siccibacter and Cronobacter, preferably from Pantoea, Franconibacter, Siccibacter and Cronobacter. Alternatively, the polypeptide exhibiting beta-carotene hydroxylase activity may be selected from the group consisting of beta-carotene hydroxylases from organisms belonging to the genera Pantoea, Marchantia, Haematococcus, Tagetes, Brevundimonas and Paracoccus, preferably from organisms belonging to the genus Pantoea or Paracoccus, more preferably from organisms belonging to the genus Pantoea.

In particular, the Pantoea bacterium may be selected from the group consisting of Pantoea agglomerans, Pantoea ananatis and Pantoea stewartii, more preferably from Pantoea agglomerans and Pantoea ananatis, and even more preferably is Pantoea agglomerans.

In an embodiment, the polypeptide exhibiting beta-carotene hydroxylase activity is selected from the group consisting of beta-carotene hydroxylases from Marchantia polymorpha (NCBI Accession number: BAR43283; SEQ ID NO: 11), Pantoea agglomerans (NCBI Accession number: AAA64983; SEQ ID NO: 13), Haematococcus pluvialis (NCBI Accession number: AAO53295; SEQ ID NO: 15), Brevundimonas sp. SD212 (NCBI Accession number: BAD99414; SEQ ID NO: 17), Pantoea stewartii (NCBI Accession number: AAN85601; SEQ ID NO: 19), Pantoea ananatis (NCBI Accession number: AER34891; SEQ ID NO: 21), Paracoccus sp. N81106 (also named Agrobacterium aurantiacum) (NCBI Accession number: BAA09592; SEQ ID NO: 23) Tagetes erecta (NCBI Accession number: AAG10430; SEQ ID NO: 25), Franconibacter pulveris (NCBI Accession number: WP_024559002.1; SEQ ID NO: 89), Siccibacter colletis (NCBI Accession number: WP_051640535.1; SEQ ID NO: 91) and Cronobacter malonaticus (NCBI Accession number: WP_032970720.1; SEQ ID NO: 93), preferably from Pantoea agglomerans, Franconibacter pulveris, Siccibacter colletis and Cronobacter malonaticus. The polypeptide exhibiting beta-carotene hydroxylase activity may also be any polypeptide exhibiting beta-carotene hydroxylase activity and having at least 60%, preferably at least 65, 70, 75, 80, 85, 90, 95, 96, 97, 98 or 99%, identity to any beta-carotene hydroxylase listed above.

In a preferred embodiment, the polypeptide exhibiting beta-carotene hydroxylase activity is selected from the group consisting of

a) a polypeptide comprising, or consisting of, an amino acid sequence selected from the group consisting of SEQ ID NO: 11, 13, 15, 17, 19, 21, 23, 25, 89, 91 and 93, preferably selected from the group consisting of SEQ ID NO: 13, 89, 91 and 93;

b) a polypeptide exhibiting beta-carotene hydroxylase activity and having an amino acid sequence having at least 60%, preferably at least 65, 70, 75, 80, 85, 90, 95, 96, 97, 98 or 99%, identity to SEQ ID NO: 11, 13, 15, 17, 19, 21, 23, 25, 89, 91 or 93, preferably to SEQ ID NO: 13, 89, 91 or 93;

c) a polypeptide exhibiting beta-carotene hydroxylase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 12, 14, 16, 18, 20, 22, 24, 26, 90, 92 and 94, preferably selected from the group consisting of SEQ ID NO: 14, 90, 92 and 94;

d) a polypeptide exhibiting beta-carotene hydroxylase activity and which is encoded by a nucleic acid sequence which is capable of hybridizing under medium/high stringency, preferably high or very high, conditions with (i) a nucleotide sequence selected from the group consisting of SEQ ID NO: 12, 14, 16, 18, 20, 22, 24, 26, 90, 92 and 94, preferably selected from the group consisting of SEQ ID NO: 14, 90, 92 and 94 (ii) its complementary strand, or (iii) a subsequence of (i) or (ii).

In a particular embodiment, the polypeptide exhibiting beta-carotene hydroxylase activity is selected from the group consisting of

a) a polypeptide comprising, or consisting of, an amino acid sequence selected from the group consisting of SEQ ID NO: 11, 13, 15, 17, 19, 21, 23 and 25;

b) a polypeptide exhibiting beta-carotene hydroxylase activity and having an amino acid sequence having at least 60%, preferably at least 65, 70, 75, 80, 85, 90, 95, 96, 97, 98 or 99%, identity to SEQ ID NO: 11, 13, 15, 17, 19, 21, 23 or 25;

c) a polypeptide exhibiting beta-carotene hydroxylase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 12, 14, 16, 18, 20, 22, 24 and 26;

d) a polypeptide exhibiting beta-carotene hydroxylase activity and which is encoded by a nucleic acid sequence which is capable of hybridizing under medium/high stringency, preferably high or very high, conditions with (i) a nucleotide sequence selected from the group consisting of SEQ ID NO: 12, 14, 16, 18, 20, 22, 24 and 26, (ii) its complementary strand, or (iii) a subsequence of (i) or (ii).

In a more particular embodiment, the polypeptide exhibiting beta-carotene hydroxylase activity is selected from the group consisting of polypeptides comprising, or consisting of, an amino acid sequence selected from the group consisting of SEQ ID NO: 11, 13, 15, 17, 19, 21, 23, 25, 89, 91 and 93, preferably selected from the group consisting of SEQ ID NO: 13, 89, 91 and 93, and alternatively from SEQ ID NO: 11, 13, 15, 17, 19, 21, 23 and 25, and polypeptides exhibiting beta-carotene hydroxylase activity and having at least 60%, preferably at least 65, 70, 75, 80, 85, 90, 95, 96, 97, 98 or 99%, identity to SEQ ID NO: 11, 13, 15, 17, 19, 21, 23, 25, 89, 91 or 93, preferably to SEQ ID NO: 13, 89, 91 or 93, and alternatively to SEQ ID NO: 11, 13, 15, 17, 19, 21, 23 or 25. Preferably, the polypeptide exhibiting beta-carotene hydroxylase activity is selected from the group consisting of polypeptides comprising, or consisting of, an amino acid sequence selected from the group consisting of SEQ ID NO: 11, 13 and 15, and polypeptides exhibiting beta-carotene hydroxylase activity and having at least 60%, preferably at least 65, 70, 75, 80, 85, 90, 95, 96, 97, 98 or 99%, identity to SEQ ID NO: 11, 13 or 15. More preferably, the polypeptide exhibiting beta-carotene hydroxylase activity is selected from the group consisting of a polypeptide comprising, or consisting of, an amino acid sequence of SEQ ID NO: 13, and polypeptides exhibiting beta-carotene hydroxylase activity and having at least 60%, preferably at least 65, 70, 75, 80, 85, 90, 95, 96, 97, 98 or 99%, identity to SEQ ID NO: 13. In a preferred embodiment, the polypeptide exhibiting beta-carotene hydroxylase activity comprises, or consists of, the amino acid sequence of SEQ ID NO: 13.

The recombinant Deinococcus bacterium may further comprise a nucleic acid sequence encoding a polypeptide exhibiting beta-carotene ketolase activity (CrtW or CrtO). Preferably, said recombinant bacterium exhibits beta-carotene ketolase activity when cultured at a temperature between 42° C. and 50° C., preferably between 42 and 48° C., and more preferably at about 42° C., 45° C. or 48° C.

In an embodiment, the nucleic acid sequence encoding a polypeptide exhibiting beta-carotene ketolase activity is an endogenous nucleic acid. Preferably, the polypeptide exhibiting beta-carotene ketolase activity is a CrtO-type ketolase. Examples of Deinococcus bacteria CrtO include, but are not limited to, crtO of D. geothermalis (NCBI accession number: ABF46602; SEQ ID NO: 47), D. radiodurans (NCBI accession number: AAF09686), D. deserti (NCBI accession number: ACO47789), Deinococcus 20 murrayi (NCBI accession number: WP_027460237.1; SEQ ID NO: 95) and Deinococcus maricopensis (NCBI accession number: WP_043817519.1; SEQ ID NO: 97). The gene encoding CrtO in the recombinant Deinococcus bacterium of the invention may be easily identified using routine methods, for example based on homology with the nucleic acid encoding any of the above identified CrtO enzymes.

In another embodiment, the nucleic acid sequence encoding a polypeptide exhibiting beta-carotene ketolase activity is a heterologous nucleic acid.

The polypeptide exhibiting beta-carotene ketolase activity may be any known beta-carotene ketolase, in particular selected from known bacterial or algal beta-carotene ketolases, preferably from bacterial beta-carotene ketolases, more preferably from beta-carotene ketolases from Gram negative bacteria.

In particular, the polypeptide exhibiting beta-carotene ketolase activity may be selected from the group consisting of beta-carotene ketolases from organisms belonging to the genera Deinococcus, Brevundimonas, Paracoccus, Haematococcus, Erythrobacter, Sphingomonas, Bradyrhizobium, Nostoc and Chlamydomonas, preferably from organisms belonging to the genera Deinococcus, Brevundimonas and Paracoccus.

In a particular embodiment, the polypeptide exhibiting beta-carotene ketolase activity is a CrtO-type ketolase from a Deinococcus bacterium, preferably from D. geothermalis, D. murrayi, D. maricopensis, D. radiodurans, D. deserti, D. grandis, D. aquaticus, D. depolymerans, more preferably from D. geothermalis, D. murrayi, D. radiodurans, D. deserti, D. grandis, D. aquaticus, D. depolymerans, and even more preferably from D. geothermalis.

In another particular embodiment, the polypeptide exhibiting beta-carotene ketolase activity is a CrtO-type ketolase from a thermophilic Deinococcus bacterium, preferably from D. geothermalis, D murrayi and D. maricopensis.

In an embodiment, the polypeptide exhibiting beta-carotene ketolase activity is selected from the group consisting of beta-carotene ketolases from Brevundimonas sp. SD212 (NCBI Accession number: BAD99406; SEQ ID NO: 27), Paracoccus sp. PC1 (Uniprot Accession number: Q44261; SEQ ID NO: 29), Agrobacterium aurantiacum or Paracoccus sp. N81106 (NCBI Accession number: 2111222A; SEQ ID NO: 31), Paracoccus haeundaensis (SEQ ID NO: 67), Paracoccus sanguinis (Paracoccus sp. 39524) (SEQ ID NO: 69), Paracoccus sphaerophysae (SEQ ID NO: 71), Haematococcus pluvialis (NCBI Accession number: CAA60478; SEQ ID NO: 33), Erythrobacter litoralis (NCBI Accession number: WP_011413632; SEQ ID NO: 35), Sphingomonas sp. PB304 (NCBI Accession number: AIG94831; SEQ ID NO: 37), Sphingomonas sp. SRS2 (SEQ ID NO: 73), Bradyrhizobium sp. ORS 278 (NCBI Accession number: AAF78203; SEQ ID NO: 39), Nostoc sp. PCC 7120 (NCBI Accession number: BAB74888; SEQ ID NO: 41), Chlamydomonas reinhardtii (Uniprot Accession number: Q4VKB4; SEQ ID NO: 43, or its truncated form SEQ ID NO: 45, Zhong et al. J Exp Bot. 2011 June; 62(10):3659-69), Deinococcus murrayi (NCBI Accession number: WP_027460237.1; SEQ ID NO: 95), Deinococcus maricopensis (NCBI Accession number: WP_043817519.1; SEQ ID NO: 97) and Deinococcus geothermalis (NCBI Accession number: ABF46602; SEQ ID NO: 47), preferably selected from the group consisting of beta-carotene ketolases from Brevundimonas sp. SD212 (SEQ ID NO: 27), Paracoccus sp. PC1 (SEQ ID NO: 29), Agrobacterium aurantiacum or Paracoccus sp. N81106 (SEQ ID NO: 31), Paracoccus haeundaensis (SEQ ID NO: 67), Paracoccus sanguinis (Paracoccus sp. 39524) (SEQ ID NO: 69), Paracoccus sphaerophysae (SEQ ID NO: 71), Haematococcus pluvialis (SEQ ID NO: 33), Erythrobacter litoralis (SEQ ID NO: 35), Sphingomonas sp. PB304 (SEQ ID NO: 37), Sphingomonas sp. SRS2 (SEQ ID NO: 73), Bradyrhizobium sp. ORS 278 (SEQ ID NO: 39), Nostoc sp. PCC 7120 (SEQ ID NO: 41), Chlamydomonas reinhardtii (SEQ ID NO: 43, or its truncated form SEQ ID NO: 45, Zhong et al. J Exp Bot. 2011 June; 62(10):3659-69) and Deinococcus geothermalis (SEQ ID NO: 47). Preferably, the polypeptide exhibiting beta-carotene ketolase activity is selected from the group consisting of beta-carotene ketolases from Paracoccus sp. N81106 and Deinococcus geothermalis, more preferably from Deinococcus geothermalis. The polypeptide exhibiting beta-carotene ketolase activity may also be any polypeptide exhibiting beta-carotene ketolase activity and having at least 60%, preferably at least 65, 70, 75, 80, 85, 90, 95, 96, 97, 98 or 99%, identity to any beta-carotene ketolase listed above. In a particular embodiment, the polypeptide exhibiting beta-carotene ketolase activity is selected from the group consisting of beta-carotene ketolases from Paracoccus sp. N81106, Deinococcus geothermalis, Deinococcus murrayi, and Deinococcus maricopensis, more preferably from Deinococcus geothermalis. The polypeptide exhibiting beta-carotene ketolase activity may also be any polypeptide exhibiting beta-carotene ketolase activity and having at least 60%, preferably at least 65, 70, 75, 80, 85, 90, 95, 96, 97, 98 or 99%, identity to any beta-carotene ketolase listed above.

In a particular embodiment, the polypeptide exhibiting beta-carotene ketolase activity is selected from the group consisting of

a) a polypeptide comprising, or consisting of, an amino acid sequence selected from the group consisting of SEQ ID NO: 27, 29, 31, 33, 35, 37, 39, 41, 43, 45, 47, 67, 69, 71, 73, 95 and 97;

b) a polypeptide exhibiting beta-carotene ketolase activity and having an amino acid sequence having at least 60%, preferably at least 65, 70, 75, 80, 85, 90, 95, 96, 97, 98 or 99%, identity to SEQ ID NO: 27, 29, 31, 33, 35, 37, 39, 41, 43, 45, 47, 67, 69, 71, 73, 95 or 97;

c) a polypeptide exhibiting beta-carotene ketolase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 28, 30, 32, 34, 36, 38, 40, 42, 44, 46, 48, 68, 70, 72, 74, 96 and 98;

d) a polypeptide exhibiting beta-carotene ketolase activity and which is encoded by a nucleic acid sequence which is capable of hybridizing under medium/high stringency, preferably high or very high, conditions with (i) a nucleotide sequence selected from the group consisting of SEQ ID NO: 28, 30, 32, 34, 36, 38, 40, 42, 44, 46, 48, 68, 70, 72, 74, 96 and 98 (ii) its complementary strand, or (iii) a subsequence of (i) or (ii).

In another particular embodiment, the polypeptide exhibiting beta-carotene ketolase activity is selected from the group consisting of

a) a polypeptide comprising, or consisting of, an amino acid sequence selected from the group consisting of SEQ ID NO: 27, 29, 31, 33, 35, 37, 39, 41, 43, 45, 47, 67, 69, 71 and 73;

b) a polypeptide exhibiting beta-carotene ketolase activity and having an amino acid sequence having at least 60%, preferably at least 65, 70, 75, 80, 85, 90, 95, 96, 97, 98 or 99%, identity to SEQ ID NO: 27, 29, 31, 33, 35, 37, 39, 41, 43, 45, 47, 67, 69, 71 and 73;

c) a polypeptide exhibiting beta-carotene ketolase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 28, 30, 32, 34, 36, 38, 40, 42, 44, 46, 48, 68, 70, 72 and 74;

d) a polypeptide exhibiting beta-carotene ketolase activity and which is encoded by a nucleic acid sequence which is capable of hybridizing under medium/high stringency, preferably high or very high, conditions with (i) a nucleotide sequence selected from the group consisting of SEQ ID NO: 28, 30, 32, 34, 36, 38, 40, 42, 44, 46, 48, 68, 70, 72 and 74, (ii) its complementary strand, or (iii) a subsequence of (i) or (ii).

In a more particular embodiment, the polypeptide exhibiting beta-carotene ketolase activity is selected from the group consisting of polypeptides comprising, or consisting of, an amino acid sequence selected from the group consisting of SEQ ID NO: 27, 29, 31, 33, 35, 37, 39, 41, 43, 45, 47, 67, 69, 71 and 73, and polypeptides exhibiting beta-carotene ketolase activity and having at least 60%, preferably at least 65, 70, 75, 80, 85, 90, 95, 96, 97, 98 or 99%, identity to SEQ ID NO: 27, 29, 31, 33, 35, 37, 39, 41, 43, 45, 47, 67, 69, 71 and 73. Preferably, the polypeptide exhibiting beta-carotene ketolase activity is selected from the group consisting of polypeptides comprising, or consisting of, an amino acid sequence selected from the group consisting of SEQ ID NO: 27, 29, 31 and 47, and polypeptides exhibiting beta-carotene ketolase activity and having at least 60%, preferably at least 65, 70, 75, 80, 85, 90, 95, 96, 97, 98 or 99%, identity to SEQ ID NO: 27, 29, 31 or 47. Preferably, the polypeptide exhibiting beta-carotene ketolase activity is selected from the group consisting of polypeptides comprising, or consisting of, an amino acid sequence selected from the group consisting of SEQ ID NO: 27, 31 and 47, and polypeptides exhibiting beta-carotene ketolase activity and having at least 60%, preferably at least 65, 70, 75, 80, 85, 90, 95, 96, 97, 98 or 99%, identity to SEQ ID NO: 27, 31 or 47. More preferably, the polypeptide exhibiting beta-carotene ketolase activity is selected from the group consisting of a polypeptide comprising, or consisting of, an amino acid sequence of SEQ ID NO: 47 or 31, and polypeptides exhibiting beta-carotene ketolase activity and having at least 60%, preferably at least 65, 70, 75, 80, 85, 90, 95, 96, 97, 98 or 99%, identity to SEQ ID NO: 47 or 31. Alternatively, the polypeptide exhibiting beta-carotene ketolase activity is selected from the group consisting of a polypeptide comprising, or consisting of, an amino acid sequence of SEQ ID NO: 31, 47, 95 or 97 and polypeptides exhibiting beta-carotene ketolase activity and having at least 60%, preferably at least 65, 70, 75, 80, 85, 90, 95, 96, 97, 98 or 99%, identity to SEQ ID NO: 31, 47, 95 or 97, preferably selected from the group consisting of a polypeptide comprising, or consisting of, an amino acid sequence of SEQ ID NO: 47, 95 or 97 and polypeptides exhibiting beta-carotene ketolase activity and having at least 60%, preferably at least 65, 70, 75, 80, 85, 90, 95, 96, 97, 98 or 99%, identity to SEQ ID NO: 47, 95 or 97.

In a preferred embodiment, the polypeptide exhibiting beta-carotene ketolase activity is selected from the group consisting of a polypeptide comprising, or consisting of, the amino acid sequence of SEQ ID NO: 47 and polypeptides exhibiting beta-carotene ketolase activity and having at least 60%, preferably at least 65, 70, 75, 80, 85, 90, 95, 96, 97, 98 or 99%, identity to SEQ ID NO: 47. Preferably, the polypeptide exhibiting beta-carotene ketolase activity comprises, or consists of, the amino acid sequence of SEQ ID NO: 47.

In embodiments wherein the nucleic acid sequence encoding a polypeptide exhibiting beta-carotene ketolase activity is an endogenous nucleic acid, the endogenous gene is preferably overexpressed.

As described below, in some embodiments, genes encoding LmbE like protein, carotenoid 3′,4′-desaturase (CrtD), glucosyltransferase (CruC), acyltransferase (CruD) and/or C-1′,2′ hydratase (CruF), are inactivated in order to increase the yield of astaxanthin and precursors thereof. In Deinococcus, these genes belong to an operon also comprising the CrtO gene. In order to facilitate genetic manipulation, the entire operon, i.e. the operon corresponding to the operon extending from gene DGEO_RS14350 (old locus tag: Dgeo_2305) to gene DGEO_RS14375 (old locus tag: Dgeo_2310) (NCBI Genbank: NC_008025.1), may be deleted. The CrtO gene may be then reinserted in the genome in the same locus or in another place. In this case, the CrtO gene is advantageously placed under the control of a stronger promoter than the native one. Alternatively, all genes of the operon, except the CrtO gene, may be inactivated, preferably deleted.

In an embodiment, the recombinant bacterium of the invention comprises a heterologous nucleic acid sequence encoding a polypeptide exhibiting lycopene cyclase activity (CrtY).

In another embodiment, the recombinant bacterium of the invention comprises a heterologous nucleic acid sequence encoding a polypeptide exhibiting lycopene cyclase activity (CrtY) and a heterologous nucleic acid sequence encoding a polypeptide exhibiting beta-carotene hydroxylase activity (CrtZ). In this embodiment, the recombinant bacterium of the invention may further comprise an endogenous gene encoding a polypeptide exhibiting beta-carotene ketolase activity, preferably a CrtO enzyme.

In another embodiment, the recombinant bacterium of the invention comprises a heterologous nucleic acid sequence encoding a polypeptide exhibiting lycopene cyclase activity (CrtY) and a nucleic acid sequence encoding a polypeptide exhibiting beta-carotene ketolase activity (CrtW or CrtO). Optionally, the recombinant bacterium of the invention may comprise a nucleic acid sequence encoding a CrtO-type ketolase and a nucleic acid sequence encoding a CrtW-type ketolase.

In a further embodiment, the recombinant bacterium of the invention comprises a heterologous nucleic acid sequence encoding a polypeptide exhibiting lycopene cyclase activity (CrtY), a heterologous nucleic acid sequence encoding a polypeptide exhibiting beta-carotene hydroxylase activity (CrtZ) and a nucleic acid sequence encoding a polypeptide exhibiting beta-carotene ketolase activity (CrtW or CrtO). Optionally, the recombinant bacterium of the invention may comprise a nucleic acid sequence encoding a CrtO-type ketolase and a nucleic acid sequence encoding a CrtW-type ketolase.

In a further embodiment, the recombinant bacterium of the invention comprises a heterologous nucleic acid sequence encoding a polypeptide exhibiting beta-carotene hydroxylase activity (CrtZ) and a nucleic acid sequence encoding a polypeptide exhibiting beta-carotene ketolase activity (CrtW or CrtO). Optionally, the recombinant bacterium of the invention may comprise a nucleic acid sequence encoding a CrtO-type ketolase and a nucleic acid sequence encoding a CrtW-type ketolase. In this embodiment, the recombinant bacterium comprises an endogenous nucleic acid sequence encoding a polypeptide exhibiting the lycopene cyclase activity (CrtY).

The nucleic acids may encode any lycopene cyclase, beta-carotene hydroxylase or beta-carotene ketolase as described above.

Preferably, in these embodiments,
the polypeptide exhibiting lycopene cyclase activity (CrtY) is selected from the group consisting of polypeptides comprising, or consisting of, an amino acid sequence selected from the group consisting of SEQ ID NO: 1 and 7, preferably SEQ ID NO: 1, and polypeptides exhibiting lycopene cyclase activity and having at least 60%, preferably at least 65, 70, 75, 80, 85, 90, 95, 96, 97, 98 or 99%, identity to SEQ ID NO: 1 or 7, preferably SEQ ID NO: 1; and/or
the polypeptide exhibiting beta-carotene hydroxylase activity (CrtZ) is selected from the group consisting of a polypeptide comprising, or consisting of, an amino acid sequence of SEQ ID NO: 13, and polypeptides exhibiting beta-carotene hydroxylase activity and having at least 60%, preferably at least 65, 70, 75, 80, 85, 90, 95, 96, 97, 98 or 99%, identity to SEQ ID NO: 13; and/or
the polypeptide exhibiting beta-carotene ketolase activity (CrtW or CrtO) is selected from the group consisting of polypeptides comprising, or consisting of, an amino acid sequence selected from the group consisting of SEQ ID NO: 27, 29, 31 and 47, preferably from the group consisting of SEQ ID NO: 27, 31 and 47, more preferably SEQ ID NO: 47, and polypeptides exhibiting beta-carotene ketolase activity and having at least 60%, preferably at least 65, 70, 75, 80, 85, 90, 95, 96, 97, 98 or 99%, identity to SEQ ID NO: 27, 29, 31 or 47, preferably from the group consisting of SEQ ID NO: 27, 31 and 47, more preferably SEQ ID NO: 47.

In other preferred embodiments,
the polypeptide exhibiting lycopene cyclase activity (CrtY) is selected from the group consisting of polypeptides comprising, or consisting of, an amino acid sequence selected from the group consisting of SEQ ID NO: 1, 7, 9, 75, 77, 79, 81, 83, 85 and 87, and polypeptides exhibiting lycopene cyclase activity and having at least 60%, preferably at least 65, 70, 75, 80, 85, 90, 95, 96, 97, 98 or 99%, identity to SEQ ID NO: 1, 7, 9, 75, 77, 79, 81, 83, 85 or 87; and/or
the polypeptide exhibiting beta-carotene hydroxylase activity (CrtZ) is selected from the group consisting of a polypeptide comprising, or consisting of, an amino acid sequence of SEQ ID NO: 13, 89, 91 and 93, and polypeptides exhibiting beta-carotene hydroxylase activity and having at least 60%, preferably at least 65, 70, 75, 80, 85, 90, 95, 96, 97, 98 or 99%, identity to SEQ ID NO: 13, 89, 91 or 93; and/or
the polypeptide exhibiting beta-carotene ketolase activity (CrtW or CrtO) is selected from the group consisting of polypeptides comprising, or consisting of, an amino acid sequence selected from the group consisting of SEQ ID NO: 31, 47, 95 and 97, preferably from the group consisting of SEQ ID NO: 31 and 47, and more preferably SEQ ID NO: 47, and polypeptides exhibiting beta-carotene ketolase activity and having at least 60%, preferably at least 65, 70, 75, 80, 85, 90, 95, 96, 97, 98 or 99%, identity to SEQ ID NO: 31, 47, 95 and 97, preferably to SEQ ID NO: 31 or 47, more preferably to SEQ ID NO: 47.

Nucleic acid sequences encoding lycopene cyclase, beta-carotene hydroxylase and beta-carotene ketolase may be comprised in one or several expression cassettes. Each expression cassette may comprise CrtY, CrtZ and/or CrtW/O genes. In particular, the recombinant Deinococcus bacterium of the invention may comprise an expression cassette comprising (i) CrtY, (ii) CrtZ, (iii) CrtW/O, (iv) CrtY and CrtZ, (v) CrtY and CrtW/O, (vi) CrtZ and CrtW/O, or (vii) CrtY, CrtZ and CrtW/O genes. In embodiments wherein the expression cassette comprise several genes, these genes may be expressed under the control of one or several promoters. In particular, each gene may be expressed under the control of a distinct promoter. These expression cassettes may be integrated into the genome of the bacterium or may be maintained in an episomal form into an expression vector. In embodiments wherein the expression cassette(s) is(are) maintained in an episomal form, the expression vector may be present in the bacterium in one or several copies, depending on the nature of the origin of replication.

Preferably, the expression cassette(s) is(are) integrated into the genome of the bacterium. One or several copies of genes encoding lycopene cyclase (CrtY), beta-carotene hydroxylase (CrtZ) and/or beta-carotene ketolase (CrtW/O) may be introduced into the genome by methods of recombination, known to the expert in the field, including gene replacement. In an embodiment, the same number of copies of each gene is introduced into the genome.

The expression cassette(s) may be integrated into the genome in order to inactivate target genes. In a particular embodiment, the expression cassette is integrated into the genome in order to inactivate LmbE like protein encoding gene, carotenoid 3′,4′-desaturase (CrtD) gene, glucosyltransferase (CruC) gene, acyltransferase (CruD) gene and/or C-1′,2′ hydratase (CruF) gene, and optionally carotene ketolase (CrtO) gene. In another embodiment, the expression cassette is integrated into the genome in order to inactivate the phosphotransacetylase (pta) gene. Targeted genes may be replaced or inactivated by the insertion of the cassette.

Alternatively, or in addition, the expression cassette(s) may be integrated into the genome in a non-coding sequence, e.g. an insertion sequence (IS) (see for example the international patent application WO 2015/092013).

Expression cassettes useful in the present invention comprise at least one nucleic acid sequence encoding lycopene cyclase, beta-carotene hydroxylase and/or beta-carotene ketolase, operably linked to one or more control sequences, typically a transcriptional promoter and a transcription terminator, that direct the expression of said gene(s).

The control sequence may include a promoter that is recognized by the host cell. The promoter contains transcriptional control sequences that mediate the expression of the enzyme. The promoter may be any polynucleotide that shows transcriptional activity in the Deinococcus bacterium. The promoter may be a native or heterologous promoter. Preferred promoters are native and Deinococcus promoter. In this regard, various promoters have been studied and used for gene expression in Deinococcus bacteria. Examples of suitable promoters include PtufA and PtufB promoters from the translation elongation factors Tu genes tufA (e.g., D. radiodurans: DR_0309) and tufB (e.g., D. radiodurans: DR_2050), the promoter of the resU gene located in pI3, the promoter region PgroESL of the groESL operon (Lecointe, et al. 2004. Mol Microbiol 53: 1721-1730; Meima et al. 2001. J Bacteriol 183: 3169-3175), or derivatives of such promoters. Preferably, the promoter is a strong constitutive promoter.

The control sequence may also be a transcription terminator, which is recognized by Deinococcus bacteria to terminate transcription. The terminator is operably linked to the 3′-terminus of the gene. Any terminator that is functional in Deinococcus bacteria may be used in the present invention such as, for example, the terminator term116 described in Lecointe et al (Lecointe, et al. 2004. Mol Microbiol 53: 1721-1730).

Optionally, the expression cassette may also comprise a selectable marker that permits easy selection of recombinant bacteria. Typically, the selectable marker is a gene encoding antibiotic resistance or conferring auxotrophy.

The Deinococcus host cell may be transformed, transfected or transduced in a transient or stable manner. The recombinant Deinococcus bacterium of the invention may be obtained by any method known by the skilled person, such as electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation or liposome-mediated transformation.

The term “recombinant Deinococcus bacterium” also encompasses the genetically modified host cell as well as any progeny that is not identical to the parent host cell, in particular due to mutations that occur during replication.

In preferred embodiments, the recombinant bacterium of the invention is able to produce astaxanthin, or a precursor thereof, preferably beta-carotene, zeaxanthin or canthaxanthin, when cultured at a temperature between 40° C. and 50° C., preferably between 42 and 48° C., and more preferably at about 42° C., 45° C. or 48° C. Preferably, said recombinant bacterium is a thermophilic Deinococcus, more preferably Deinococcus geothermalis.

Inactivation of Pathways Competitive to the Pathway Converting Lycopene to Astaxanthin or a Precursor Thereof

In some embodiments, one or several endogenous biosynthetic pathways converting lycopene to a carotenoid distinct from astaxanthin (or one of its precursors), in particular deinoxanthin, are blocked/inactivated or reduced to improve the flow of carbon towards astaxanthin and precursors thereof.

One or several endogenous genes involved in competitive pathways may be inactivated. Genes may be inactivated by any method known by the skilled person, for example by deletion of all or part of this gene, by introducing a nonsense codon or a mutation inducing a frameshift, or by insertion of an expression cassette, e.g. an expression cassette comprising CrtY, CrtY and/or CrtW/O genes. Alternatively, the expression of one or several endogenous genes involved in competitive pathways may be reduced. This reduction may be obtained, for example, by replacing endogenous promoters by weaker promoters, such as PlexA or PamyE promoters (Meima et al. 2001. J Bacteriol 183: 3169-3175). In preferred embodiments, targeted genes are inactivated, preferably by deleting all or part of said genes, for example by gene replacement.

Examples of said genes include, but are not limited to, the genes encoding LmbE like protein, carotenoid 3′,4′-desaturase (CrtD), glucosyltransferase (CruC), acyltransferase (CruD) and C-1′,2′ hydratase (CruF). Preferably, genes encoding CrtD, CruC, CruD and CruF are deleted or inactivated. Optionally, the gene encoding CrtO is also deleted, in particular when accumulation of 3-caroten or zeaxanthin is desired.

In Deinococcus bacteria, these genes belong to an operon. For example, in D. geothermalis bacteria, this operon extends from gene DGEO_RS14350 (old locus tag: Dgeo_2305) to gene DGEO_RS14375 (old locus tag: Dgeo_2310) (NCBI Genbank: NC_008025.1). In some embodiments, the entire operon may be inactivated. In some other embodiments, one or several genes of this operon may be inactivated.

The term “CrtD” refers to an enzyme carotenoid 3′,4′-desaturase (also named methoxyneurosporene dehydrogenase) encoded by crtD gene, that catalyses the C-3′,4′-desaturation of the monocyclic precursor of deinoxanthin. Examples of crtD include, but are not limited to, crtD of D. geothermalis (NCBI accession number: ABF46598), D. radiodurans (NCBI accession number: AAF11796) and D. deserti (NCBI accession number: ACO47784). The gene encoding CrtD in the recombinant Deinococcus bacterium of the invention may be easily identified using routine methods, for example based on homology with the nucleic acid encoding any of the above identified CrtD enzymes.

The term “CruC” refers to a carotenoid glucosyltransferase encoded by cruC gene, that catalyses glycosylation on the yr end of monocyclic carotenoids. Examples of CruC include, but are not limited to, CruC of D. geothermalis (NCBI accession number: ABF46599), D. radiodurans (NCBI accession number: NP_293815) and D. deserti (NCBI accession number: ACO47785). The gene encoding CruC in the recombinant Deinococcus bacterium of the invention may be easily identified using routine methods, for example based on homology with the nucleic acid encoding any of the above identified CruC enzymes.

The term “CruD” refers to a carotenoid acyltransferase encoded by cruD gene, that catalyses acylation on the yr end of monocyclic carotenoids. Examples of CruD include, but are not limited to, CruD of D. geothermalis (NCBI accession number: ABF46600), D. radiodurans (NCBI accession number: AAF09683) and D. deserti (NCBI accession number: ACO47787). The gene encoding CruD in the recombinant Deinococcus bacterium of the invention may be easily identified using routine methods, for example based on homology with the nucleic acid encoding any of the above identified CruD enzymes.

The term “CruF” refers to a carotenoid 1′,2′ hydratase encoded by cruF gene. Examples of CruF include, but are not limited to, CruF of D. geothermalis (NCBI accession number: ABF46601), D. radiodurans (NCBI accession number: NP_293817) and D. deserti (NCBI accession number: ACO47788). The gene encoding CruF in the recombinant Deinococcus bacterium of the invention may be easily identified using routine methods, for example based on homology with the nucleic acid encoding any of the above identified CruF enzymes.

Optionally, the CrtLm lycopene β-cyclase found in Deinococcus bacteria may also be inactivated. Indeed, this enzyme exhibits only monocyclization activity and convert lycopene to γ-carotene but not to β-carotene. Examples of CrtLm include, but are not limited to, CrtLm of D. geothermalis (NCBI accession number: ABF45159) and D. radiodurans (NCBI accession number: Q9RW68). The gene encoding CrtLm in the recombinant Deinococcus bacterium of the invention may be easily identified using routine methods, for example based on homology with the nucleic acid encoding any of the above identified CrtLm enzymes.

Enhancement of the Flow of Carbon Through the Biosynthetic Pathway of Astaxanthin or Precursors Thereof

To enhance the production of astaxanthin or a precursor thereof, the recombinant bacterium of the invention may also be genetically modified in order to increase the production of lycopene.

In particular, the recombinant bacterium of the invention may be genetically modified to increase the carbon flux to isopentenyl pyrophosphate (IPP) and dimethylallyl pyrophosphate (DMAPP), to increase the conversion of IPP and DMAPP to geranylgeranyl diphosphate (GGPP), and/or to increase to conversion of GGPP to lycopene.

The carbon flux to IPP and DMAPP may be increased by enhancing the 2-C-methyl-D-erythritol 4-phosphate/1-deoxy-D-xylulose 5-phosphate (MEP/DXP) pathway. As used herein, the term “MEP pathway” or “MEP/DXP pathway” refers to the biosynthetic pathway leading to the formation of IPP and DMAPP from the condensation of pyruvate and D-glyceraldehyde 3-phosphate to 1-deoxy-D-xylulose 5-phosphate (DXP). This pathway involves the following enzymes: 1-deoxy-D-xylulose 5-phosphate synthase (EC 2.2.1.7), 1-deoxy-D-xylulose 5-phosphate reductoisomerase (EC 1.1.1.267), 2-C-methyl-D-erythritol 4-phosphate cytidylyltransferase (EC 2.7.7.60), 4-diphosphocytidyl-2-C-methyl-D-erythritol kinase (EC 2.7.1.148), 2-C-methyl-D-erythritol 2,4-cyclodiphosphate synthase (EC 4.6.1.12), 4-hydroxy-3-methylbut-2-en-1-yl diphosphate synthase (EC 1.17.7.1), 4-hydroxy-3-methylbut-2-enyl diphosphate reductase (EC 1.17.1.2), and isopentenyl-diphosphate delta-isomerase (EC 5.3.3.2).

This pathway may be enhanced by any method known by the skilled person, for example by a method described in the patent application WO 2015/189428. In particular, this pathway may be enhanced by increasing at least one enzymatic activity selected from the group consisting of DXP synthase (DXS), DXP reductoisomerase (DXR), IspD, IspE, IspF, IspG, IspH and IPP isomerase activities (IDI), preferably by increasing DXP synthase and IPP isomerase activities.

In the present invention, an enzymatic activity (e.g. DXS, DXR, IspD, IspE, IspF, IspG, IspH, IDI, FPPS, phytoene synthase or phytoene desaturase activity) may be increased by overexpression of an endogenous gene or expression of a heterologous gene, and/or expression of an improved variant of the endogenous enzyme, i.e. an enzyme that possesses at least one mutation in its sequence, in comparison with the amino acid sequence of the wild-type enzyme, said mutation leading to an increase of its activity, an increased specific catalytic activity, an increased specificity for the substrate, an increased protein or RNA stability and/or an increased intracellular concentration of the enzyme, or leading to a feedback resistant mutant.

The term “DXS” or “DXP synthase” refers to the enzyme 1-deoxy-D-xylulose 5-phosphate synthase (EC 2.2.1.7) encoded by the dxs gene. The term “DXP reductoisomerase” or “DXR” refers to the enzyme 1-deoxy-D-xylulose 5-phosphate reductoisomerase (EC 1.1.1.267) encoded by the dxr gene. The term “IspD” refers to the enzyme 2-C-methyl-D-erythritol 4-phosphate cytidylyltransferase (EC 2.7.7.60) encoded by the ispD gene. The term “IspE” refers to the enzyme 4-diphosphocytidyl-2-C-methyl-D-erythritol kinase, (EC 2.7.1.148) encoded by the ispE gene. The term “IspF” refers to the enzyme 2-C-methyl-D-erythritol 2,4-cyclodiphosphate synthase (EC 4.6.1.12) encoded by the ispF gene. The term “IspG” refers to the enzyme 4-hydroxy-3-methylbut-2-en-1-yl diphosphate synthase (EC 1.17.7.1) encoded by the ispG gene. The term “IspH” refers to the enzyme 4-hydroxy-3-methylbut-2-enyl diphosphate reductase, also named hydroxymethylbutenyl pyrophosphate reductase, (EC 1.17.1.2) encoded by the ispH gene. The term “IDI” refers to the enzyme isopentenyl-diphosphate delta-isomerase (EC 5.3.3.2) encoded by the idi gene.

Preferably, at least one gene selected from the group consisting of dxs, dxr, ispD, ispE, ispF, ispG, ispH and idi genes, is overexpressed, more preferably dxs and/or idi genes, and even more preferably dxs and idi genes. These genes may be endogenous or heterologous. dxs, dxr, ispD, ispE, ispF, ispG, ispH and idi genes of the recombinant Deinococcus bacterium of the invention may be easily identified as described in the patent application WO 2015/189428.

Optionally, the recombinant bacterium of the invention may also express a variant of a Deinococcus DXP synthase which exhibits increased activity by comparison to the wild-type enzyme. Such improved DXP synthases are described in the international patent applications WO 2015/189428 and WO 2012/052171.

In addition, or alternatively, the lycopene production may also be improved by increasing the conversion of IPP and DMAPP to GGPP. Preferably, in the recombinant bacterium of the invention, the FPP synthase activity is increased by comparison to the wild-type bacterium.

As used herein, the term “FPPS”, “FDPS” or “FPP synthase” refers to an enzyme encoded by the fdps (or crtE) gene and exhibiting farnesyl diphosphate synthase activity (EC 2.5.1.10), dimethylallyltranstransferase activity (EC 2.5.1.1) and geranylgeranyl diphosphate synthase activity (EC 2.5.1.29).

Preferably, the FPP synthase activity is increased by overexpression of an endogenous gene or expression of a heterologous fdps gene. In particular, the recombinant Deinococcus bacterium of the invention may comprise a heterologous nucleic acid encoding a polypeptide exhibiting FPP synthase activity and/or may overexpress an endogenous nucleic acid encoding a polypeptide exhibiting FPP synthase activity.

The polypeptide exhibiting FPP synthase activity may be any known FPP synthase, preferably any FPP synthase from Deinococcus bacteria.

Examples of Deinococcus FPP synthases include, but are not limited to, FPP synthases from D. geothermalis (NCBI Accession number: ABF45913; SEQ ID NO: 49), D. radiodurans (NCBI Accession number: NP_295118; SEQ ID NO: 51) and D. deserti (NCBI Accession number: ACO46371; SEQ ID NO 53). The polypeptide exhibiting FPP synthase activity may also be any polypeptide exhibiting FPP synthase activity and having at least 60%, preferably 65, 70, 75, 80, 85, 90, 95, 96, 97, 98 or 99%, identity to any FPP synthase listed above.

In a particular embodiment, the polypeptide exhibiting FPP synthase activity is selected from the group consisting of

a) a polypeptide comprising, or consisting of, an amino acid sequence selected from the group consisting of SEQ ID NO: 49, 51 and 53, preferably SEQ ID NO: 49;

b) a polypeptide exhibiting FPP synthase activity and having an amino acid sequence having at least 60%, preferably at least 65, 70, 75, 80, 85, 90, 95, 96, 97, 98 or 99%, identity to SEQ ID NO: 49, 51 or 53, preferably SEQ ID NO: 49;

c) a polypeptide exhibiting FPP synthase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 50, 52 and 54, preferably SEQ ID NO: 50;

d) a polypeptide exhibiting FPP synthase activity and which is encoded by a nucleic acid sequence which is capable of hybridizing under medium/high stringency, preferably high or very high, conditions with (i) a nucleotide sequence selected from the group consisting of SEQ ID NO: 50, 52 and 54, preferably SEQ ID NO: 50, (ii) its complementary strand, or (iii) a subsequence of (i) or (ii).

In addition, or alternatively, the lycopene production may also be improved by increasing the conversion of GGPP to lycopene. Preferably, in the recombinant bacterium of the invention, phytoene synthase and/or phytoene desaturase activities are increased by comparison to the wild-type bacterium. More preferably, in the recombinant bacterium of the invention, phytoene synthase and phytoene desaturase activities are increased.

As used herein, the term “phytoene synthase” or “CrtB” refers to an enzyme encoded by the crtB gene and exhibiting phytoene synthase activity (EC 2.5.1.32). This enzyme catalyzes the conversion of geranylgeranyl diphosphate to phytoene.

As used herein, the term “phytoene desaturase”, “phytoene dehydrogenase”, “phytoene desaturase (lycopene forming)” or “CrtI” refers to an enzyme encoded by the crtI gene and exhibiting 4-step phytoene desaturase activity (EC 1.3.99.31). This enzyme catalyzes up to four desaturation steps (cf. EC 1.3.99.28, EC 1.3.99.29 and EC 1.3.99.30) to convert phytoene to lycopene.

Preferably, the phytoene synthase and/or desaturase activities are increased by overexpression of endogenous or heterologous crtB and/or crtI gene. In particular, the recombinant Deinococcus bacterium of the invention may comprise a heterologous nucleic acid encoding a polypeptide exhibiting phytoene synthase activity and/or a heterologous nucleic acid encoding a polypeptide exhibiting phytoene desaturase activity and/or may overexpress an endogenous nucleic acid encoding a polypeptide exhibiting phytoene synthase activity and/or an endogenous nucleic acid encoding a polypeptide exhibiting phytoene desaturase activity.

The polypeptide exhibiting phytoene synthase activity may be any known phytoene synthase, preferably any phytoene synthase from Deinococcus bacteria.

Examples of Deinococcus phytoene synthases (CrtB) include, but are not limited to, phytoene synthases from D. geothermalis (NCBI Accession number: ABF44825; SEQ ID NO: 55), D. radiodurans (NCBI Accession number: NP_294586; SEQ ID NO: 57) and D. deserti (NCBI Accession number: ACO47782; SEQ ID NO 59). The polypeptide exhibiting phytoene synthase activity may also be any polypeptide exhibiting phytoene synthase activity and having at least 60%, preferably at least 65, 70, 75, 80, 85, 90, 95, 96, 97, 98 or 99%, identity to any phytoene synthase listed above.

In a particular embodiment, the polypeptide exhibiting phytoene synthase activity is selected from the group consisting of

a) a polypeptide comprising, or consisting of, an amino acid sequence selected from the group consisting of SEQ ID NO: 55, 57 and 59, preferably SEQ ID NO: 55;

b) a polypeptide exhibiting phytoene synthase activity and having an amino acid sequence having at least 60%, preferably at least 65, 70, 75, 80, 85, 90, 95, 96, 97, 98 or 99%, identity to SEQ ID NO: 55, 57 or 59, preferably SEQ ID NO: 55;

c) a polypeptide exhibiting phytoene synthase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 56, 58 and 60, preferably SEQ ID NO: 56;

d) a polypeptide exhibiting phytoene synthase activity and which is encoded by a nucleic acid sequence which is capable of hybridizing under medium/high stringency, preferably high or very high, conditions with (i) a nucleotide sequence selected from the group consisting of SEQ ID NO: 56, 58 and 60, preferably SEQ ID NO: 56, (ii) its complementary strand, or (iii) a subsequence of (i) or (ii).

The polypeptide exhibiting phytoene desaturase activity may be any known phytoene desaturase, preferably any phytoene desaturase from Deinococcus bacteria.

Examples of Deinococcus phytoene desaturase (CrtI) include, but are not limited to, phytoene desaturase from D. geothermalis (NCBI Accession number: ABF44826; SEQ ID NO: 61), D. radiodurans (NCBI Accession number: NP_294585; SEQ ID NO: 63) and D. deserti (NCBI Accession number: ACO47783; SEQ ID NO 65). The polypeptide exhibiting phytoene desaturase activity may also be any polypeptide exhibiting phytoene desaturase activity and having at least 60%, preferably at least 65, 70, 75, 80, 85, 90, 95, 96, 97, 98 or 99%, identity to any phytoene desaturase listed above.

In a particular embodiment, the polypeptide exhibiting phytoene desaturase activity is selected from the group consisting of

a) a polypeptide comprising, or consisting of, an amino acid sequence selected from the group consisting of SEQ ID NO: 61, 63 and 65, preferably SEQ ID NO: 61;

b) a polypeptide exhibiting phytoene desaturase activity and having an amino acid sequence having at least 60%, preferably at least 65, 70, 75, 80, 85, 90, 95, 96, 97, 98 or 99%, identity to SEQ ID NO: 61, 63 or 65, preferably SEQ ID NO: 61;

c) a polypeptide exhibiting phytoene desaturase activity encoded by a nucleotide sequence having at least 60%, preferably at least 65, 70, 75, 80, 85, 90, 95, 96, 97, 98 or 99%, identity to a nucleotide sequence selected from the group consisting of SEQ ID NO: 62, 64 and 66, preferably SEQ ID NO: 62;

d) a polypeptide exhibiting phytoene desaturase activity and which is encoded by a nucleic acid sequence which is capable of hybridizing under medium/high stringency, preferably high or very high, conditions with (i) a nucleotide sequence selected from the group consisting of SEQ ID NO: 62, 64 and 66, preferably SEQ ID NO: 62, (ii) its complementary strand, or (iii) a subsequence of (i) or (ii).

In a particular embodiment, the recombinant bacterium of the invention is genetically modified in order to increase the production of lycopene, to increase the conversion of IPP and DMAPP to GGPP and to increase the conversion of GGPP to lycopene. Preferably, the recombinant bacterium exhibits increased FPP synthase, DXP synthase, IPP isomerase, phytoene synthase and/or phytoene desaturase activities by comparison to the wild-type bacterium. More preferably, the recombinant bacterium exhibits increased FPP synthase, DXP synthase, IPP isomerase, phytoene synthase and phytoene desaturase activities by comparison to the wild-type bacterium.

Heterologous Carotenoid 2,2′-β-Hydroxylases

The recombinant bacterium of the invention comprising at least a heterologous nucleic acid sequence encoding a polypeptide exhibiting lycopene cyclase activity and a heterologous nucleic acid sequence encoding a polypeptide exhibiting beta-carotene hydroxylase activity, may further comprise a heterologous nucleic acid sequence encoding a polypeptide exhibiting carotenoid 2,2′-β-hydroxylase activity. Optionally, the recombinant bacterium may be also genetically modified in order to increase the production of lycopene as described above.

As used herein, the term “carotenoid 2,2′-β-hydroxylase”, “2,2′-beta-ionone ring hydroxylase” or “CrtG” refers to an enzyme encoded by a crtG gene. This enzyme catalyzes the conversion of zeaxanthin to nostoxanthin.

Examples of carotenoid 2,2′-β-hydroxylases include, but are not limited to, carotenoid 2,2′-β-hydroxylases from Sphingomonas elodea (NCBI Accession number: AEP37351), Brevundimonas vesicularis (NCBI Accession number: ABC50107), Brevundimonas aurantiaca (NCBI Accession number: ABF50965), Brevundimonas sp. SD212 (NCBI Accession number: BAD99415) and Thermosynechococcus sp. NK55a (NCBI Accession number: AHB88556). The polypeptide exhibiting carotenoid 2,2′-β-hydroxylase activity may also be any polypeptide exhibiting carotenoid 2,2′-β-hydroxylase activity and having at least 60%, preferably at least 65, 70, 75, 80, 85, 90, 95, 96, 97, 98 or 99%, identity to any carotenoid 2,2′-β-hydroxylase listed above.

Cell Extracts

In another aspect, the present invention also relates to a cell extract of the recombinant Deinococcus bacterium of the invention. As used herein, the term “cell extract” refers to any fraction obtained from a host cell, such as a cell supernatant, a cell debris, cell walls, DNA or RNA extract, protein, enzymes or enzyme preparation or any preparation derived from host cells by chemical, physical and/or enzymatic treatment, which is essentially or mainly free of living cells. The invention further relates to the use of said cell extract to produce astaxanthin, or a precursor thereof, or nostoxanthin.

Methods of Production

In a further aspect, the present invention relates to a use of a recombinant Deinococcus bacterium of the invention for producing astaxanthin or a precursor thereof.

In particular, the present invention relates to a method of producing astaxanthin or a precursor thereof comprising (i) culturing a recombinant Deinococcus bacterium according to the invention under conditions suitable to produce astaxanthin or a precursor thereof and optionally (ii) recovering said astaxanthin or precursor thereof. The method may further comprise isolating or purifying said astaxanthin or precursor thereof.

As used herein, the term “precursor of astaxanthin” includes β-carotene, echinenone, canthaxantin, adonirubin, 3′hydroxyechinenone, 3-hydroxyechinenone, β-cryptoxanthin, zeaxanthin and adonixanthin. Preferably, this term refers to beta-carotene, zeaxanthin or canthaxanthin, more preferably to zeaxanthin or canthaxanthin.

In an embodiment, the present invention relates to a method of producing β-carotene, said method comprising (i) culturing a recombinant Deinococcus bacterium according to the invention and comprising a heterologous nucleic acid sequence encoding a polypeptide exhibiting CrtY activity under conditions suitable to produce β-carotene and optionally (ii) recovering said β-carotene. The method may further comprise isolating or purifying said β-carotene.

In another embodiment, the present invention relates to a method of producing canthaxantin comprising (i) culturing a recombinant Deinococcus bacterium according to the invention and comprising a heterologous nucleic acid sequence encoding a polypeptide exhibiting CrtY activity and a nucleic acid sequence encoding a polypeptide exhibiting beta-carotene ketolase activity (CrtW/O), under conditions suitable to produce canthaxanthin, and optionally (ii) recovering said canthaxanthin. The method may further comprise isolating or purifying said canthaxanthin.

In a further embodiment, the present invention relates to a method of producing zeaxanthin comprising (i) culturing a recombinant Deinococcus bacterium according to the invention and comprising a heterologous nucleic acid sequence encoding a polypeptide exhibiting CrtY activity and a heterologous nucleic acid sequence encoding a polypeptide exhibiting CrtZ activity under conditions suitable to produce zeaxanthin, and optionally (ii) recovering said zeaxanthin. The method may further comprise isolating or purifying said zeaxanthin.

In a preferred embodiment, the present invention relates to a method of producing astaxanthin comprising (i) culturing a recombinant Deinococcus bacterium according to the invention and comprising a heterologous nucleic acid sequence encoding a polypeptide exhibiting CrtY activity, a heterologous nucleic acid sequence encoding a polypeptide exhibiting CrtZ activity and a nucleic acid sequence encoding a polypeptide exhibiting beta-carotene ketolase (CrtW/O) activity under conditions suitable to produce astaxanthin, and optionally (ii) recovering said astaxanthin. The method may further comprise isolating or purifying said astaxanthin.

In a further aspect, the present invention also relates to a use of a recombinant Deinococcus bacterium of the invention for producing nostoxanthin. In particular, it relates to a method of producing nostoxanthin comprising (i) culturing a recombinant Deinococcus bacterium according to the invention and comprising a heterologous nucleic acid sequence encoding a polypeptide exhibiting CrtY activity, a heterologous nucleic acid sequence encoding a polypeptide exhibiting CrtZ activity and a heterologous nucleic acid sequence encoding a polypeptide exhibiting CrtG activity under conditions suitable to produce nostoxanthin, and optionally (ii) recovering said nostoxanthin. The method may further comprise isolating or purifying said nostoxanthin.

All embodiments described above for the recombinant Deinococcus of the invention are also contemplated in these aspects. In particular, the recombinant Deinococcus bacterium used in a method of production of the invention may be genetically modified in order to improve the flow of carbon towards astaxanthin and precursors thereof (e.g. by blocking or reducing one or several endogenous biosynthetic pathways converting lycopene to a carotenoid distinct from astaxanthin or precursors thereof) and/or to increase the production of lycopene as described above. In particular, the recombinant may exhibit increased FPP synthase, DXP synthase, IPP isomerase, phytoene synthase and/or phytoene desaturase activities by comparison to the wild-type bacterium.

Conditions suitable to produce astaxanthin or a precursor thereof, or nostoxanthin may be easily determined by the skilled person according to the recombinant Deinococcus bacterium used. In particular, the carbon source may be selected from the group consisting of C5 sugars such as xylose and arabinose, C6 sugars such as glucose, cellobiose, saccharose and starch.

Preferably, astaxanthin or a precursor thereof, or nostoxanthin is produced from renewable, biologically derived carbon sources such as cellulosic biomass. As used herein, the term “cellulosic biomass” refers to any biomass material, preferably vegetal biomass, comprising cellulose, hemicellulose and/or lignocellulose, preferably comprising cellulose and hemicellulose. Cellulosic biomass includes, but is not limited to, plant material such as forestry products, woody feedstock (softwoods and hardwoods), agricultural wastes and plant residues (such as corn stover, shorghum, sugarcane bagasse, grasses, rice straw, wheat straw, empty fruit bunch from oil palm and date palm, agave bagasse, from tequila industry), perennial grasses (switchgrass, miscanthus, canary grass, erianthus, napier grass, giant reed, and alfalfa); municipal solid waste (MSW), aquatic products such as algae and seaweed, wastepaper, leather, cotton, hemp, natural rubber products, and food processing by-products.

Preferably, if the cellulosic biomass comprises lignocellulose, this biomass is pre-treated before hydrolysis. This pretreatment is intended to open the bundles of lignocelluloses in order to access the polymer chains of cellulose and hemicellulose. Pretreatment methods are well known by the skilled person and may include physical pretreatments (e.g. high pressure steaming, extrusion, pyrolysis or irradiation), physicochemical and chemical pretreatments (e.g. ammonia fiber explosion, treatments with alkaline, acidic, solvent or oxidizing agents) and/or biological pretreatments.

Temperature conditions can also be adapted depending on the use of mesophilic or thermophilic Deinococcus bacteria. In a preferred embodiment, the Deinococcus bacteria is a thermophilic Deinococcus, such as for example D. geothermalis, D. maricopensis or D. murrayi, preferably D. geothermalis, and the culture of the recombinant Deinococcus bacterium under conditions suitable to produce astaxanthin or a precursor thereof, or nostoxanthin is performed at a temperature comprised between 30° C. and 55° C., preferably between 35° C. and 50° C., more preferably between 40° C. and 50° C., even more preferably between 42° C. and 48° C., e.g. at about 45° C. or 48° C. In another embodiment, the Deinococcus bacteria is a mesophilic Deinococcus, such as for example, D. grandis, D. aquaticus, D. indicus, D. cellulosilyticus or D. depolymerans, and the culture of the recombinant Deinococcus bacterium under conditions suitable to produce astaxanthin or a precursor thereof, or nostoxanthin is performed at a temperature comprised between 20° C. and 40° C., preferably between 28 and 35° C., more preferably at about 30° C.

Preferably, in the method of the invention, the recombinant Deinococcus bacterium, preferably a thermophilic Deinococcus, is cultured at a temperature between 37° C. and 60° C., between 37° C. and 55° C., or between 37 and 50° C., preferably between 40° C. and 48° C., more preferably between 42° C. and 48° C., and even more preferably between 42° C. and 45° C. or between 40° C. and 45° C. In some particular embodiments, the recombinant Deinococcus bacterium is cultured at a temperature of about 40° C., 42° C., 45° C. or 48° C.

The methods of the invention may be performed in a reactor, in particular a reactor of conversion of biomass. By “reactor” is meant a conventional fermentation tank or any apparatus or system for biomass conversion, typically selected from bioreactors, biofilters, rotary biological contactors, and other gaseous and/or liquid phase bioreactors. The apparatus which can be used according to the invention can be used continuously or in batch loads. Depending on the cells used, the method may be conducted under aerobiosis, anaerobiosis or microaerobiosis.

The present invention further relates to a reactor comprising a recombinant Deinococcus bacterium of the invention, or a cell extract thereof. Preferably, the reactor further comprises a carbon source, more preferably a biologically derived carbon source such as cellulosic biomass. The invention further relates to the use of said reactor to produce astaxanthin, or a precursor thereof, or nostoxanthin.

The invention also relates to the use of a recombinant Deinococcus bacterium of the invention to produce astaxanthin, and/or a precursor thereof, or nostoxanthin. Preferably, the invention relates to the use of a recombinant Deinococcus bacterium of the invention to produce beta-carotene, zeaxanthin and/or canthaxanthin.

The present invention also relates to a composition comprising a recombinant Deinococcus bacterium of the invention or an extract thereof and the use of said composition to produce astaxanthin or a precursor thereof, or nostoxanthin. Preferably, the composition further comprises a carbon source, more preferably a biologically derived carbon source such as cellulosic biomass.

The invention also relates to astaxanthin or a precursor thereof, or nostoxanthin, preferably isolated or purified astaxanthin, precursor or nostoxanthin, obtainable or obtained by a method of the invention.

All embodiments described above for the recombinant Deinococcus bacterium of the invention are also contemplated in these aspects.

Further aspects and advantages of the present invention will be described in the following examples, which should be regarded as illustrative and not limiting.

### Examples

### Example 1: Production of γ-Carotene

A Deinococcus geothermalis strain was genetically engineered to produce gamma-carotene. This recombinant strain was obtained by disrupting a part of the carotenoid pathway. Indeed, the operon containing genes encoding LmbE like protein, carotenoid 3′,4′-desaturase (CrtD), glucosyltransferase (CruC), acyltransferase (CruD), C-1′,2′ hydratase (CruF) and carotene ketolase (CrtO), i.e. the operon extending from gene DGEO_RS14350 (old locus tag: Dgeo_2305) to gene DGEO_RS14375 (old locus tag: Dgeo_2310) (cf. NCBI Genbank: NC_008025.1, Deinococcus geothermalis, complete genome), was knockout. Moreover, the endogenous farnesyl pyrophosphate synthase (FPPS, E.C. 2.5.1.1, 2.5.1.10, 2.5.1.29) gene was overexpressed by replacing the endogenous fdps gene (DGEO_RS10825 (old locus tag: Dgeo_1618; NCBI Genbank: ABF45913) with a cassette comprising said gene placed under the control of a constitutive promoter. The resulting constructs were checked by sequencing. This strain was named strain A.

To make seed cultures, individual colonies were picked to inoculate 25 ml of CMG2% medium (Peptone 2 g/L; Yeast Extract 5 g/L; Glucose 55 mM (20 g/L); MOPS acid 40 mM; NH₄Cl 20 mM; NaOH 10 mM; KOH 10 mM; CaCl₂.2H₂O 0.5 μM; Na₂SO₄.10H₂O 0.276 mM; MgCl₂.6H₂O 0.528 mM; (NH₄)₆(Mo₇)O₂₄.4H₂O 3 nM; H₃BO₃ 0.4 μM; CoCl₂.6H₂O 30 nM; CuSO₄.5H₂O 10 nM; MnCl₂ 0.25 μM; ZnSO₄.7H₂O 10 nM; D-Biotin 1 μg/L; Niacin (nicotinic acid) 1 μg/L; B6 vitamin 1 g/L; B vitamin; FeCl₃ 20 μM; Sodium Citrate.2H₂O 20 μM; K₂HPO₄ 5.7 mM) containing 2% glucose as the main carbon source, and cultured at 45° C. and 250 rpm overnight. Seed from log phase of growth was then inoculated into 25 ml of the same fresh medium at an initial optical density at 600 nm (OD600) of 0.4. This second seed culture was cultured at 45° C. and 250 rpm overnight. The cultures for gamma-carotene production were performed at 45° C. and 250 rpm for 24 h from log phase of growth inoculated into 25 ml of mineral define medium (NH₄)₂SO₄<100 mM; NaH₂PO₄.H₂O<10 mM; KCl<10 mM; Na₂SO₄<10 mM; Acide citrique<30 mM; MgCl₂.6H₂O<10 mM; CaCl₂.2H₂O<10 mM; ZnCl₂<50 mg/L; FeSO₄.7H₂O<50 mg/L; MnCl₂.4H₂O<50 mg/L; CuSO₄<50 mg/L; CoCl₂.6H₂O<50 mg/L; H₃BO₃<5 mg/L; MES<200 mM; (NH₄)₆Mo₇O₂₄.4H₂O<0.5 mM; Glucose<30 g/L (166 mM) at an initial optical density at 600 nm (OD600) of 0.4. After 24 h of culture 1 mL of culture was centrifuged and carotenoid extraction was done by mixing 1 mL of ethanol with pellet.

The ethanol phase was analyzed by HPLC (Column c18 poroshell agilent 150 mm*2.1 mm*2.7 um, mobile phase acetonitrile/methanol/ethyl acetate).

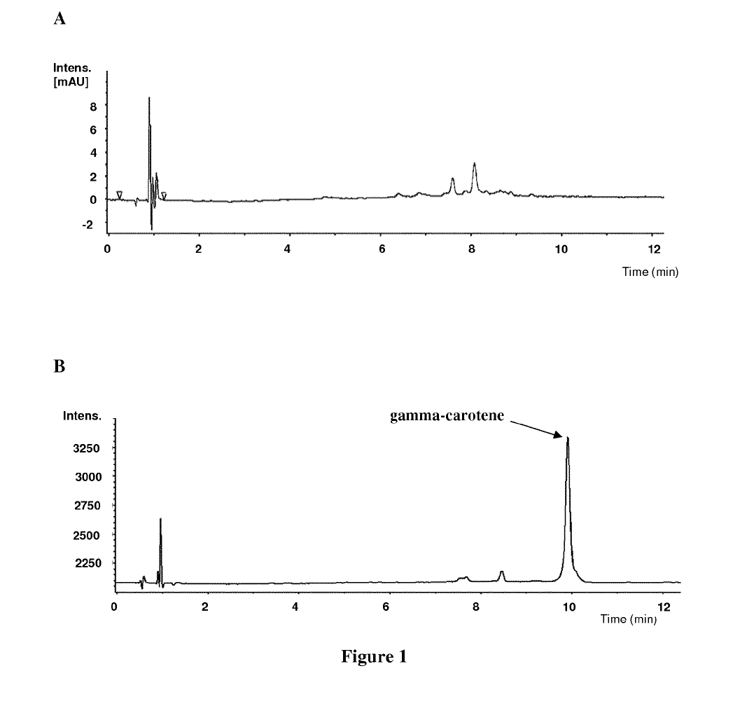

As shown in FIG. 1B, the recombinant D. geothermalis having a disrupted carotenoid pathway and overexpressing FPPS, produced and accumulated gamma carotene. On the contrary, no gamma carotene could be detected on the HPLC spectrum of the wild-type D. geothermalis strain (cf. FIG. 1A).

### Example 2: Production of β-Carotene

A Deinococcus geothermalis strain was genetically engineered to produce beta-carotene. This recombinant Deinococcus geothermalis strain was obtained by further modifying strain A of example 1. Indeed, the gene encoding the lycopene beta-cyclase (CrtY, cyclizing lycopene into beta-carotene) from Pantoea ananatis (SEQ ID NO: 1) or from Deinococcus deserti (SEQ ID NO: 7) was inserted into the chromosome (replacing the phosphotransacetylase (pta) gene (DGEO_RS02840 NCBI Genbank: NC_008025.1) or the carotenoid operon extending from gene DGEO_RS14350 to gene DGEO_RS14375) and placed under the control of a constitutive promoter. The resulting constructs were checked by sequencing. The strain expressing CrtY from Pantoea ananatis was named strain B and the strain expressing CrtY from Deinococcus deserti was named strain C.

To make seed cultures, individual colonies were picked to inoculate 25 ml of CMG2% medium (Peptone 2 g/L; Yeast Extract 5 g/L; Glucose 55 mM (20 g/L); MOPS acid 40 mM; NH₄Cl 20 mM; NaOH 10 mM; KOH 10 mM; CaCl₂.2H₂O 0.5 μM; Na₂SO₄.10H₂O 0.276 mM; MgCl₂.6H₂O 0.528 mM; (NH₄)₆(Mo₇)O₂₄.4H₂O 3 nM; H₃BO₃ 0.4 μM; CoCl₂.6H₂O 30 nM; CuSO₄.5H₂O 10 nM; MnCl₂ 0.25 PM; ZnSO₄.7H₂O 10 nM; D-Biotin 1 μg/L; Niacin (nicotinic acid) 1 μg/L; B6 vitamin 1 g/L; BI vitamin; FeCl₃ 20 μM; Sodium Citrate.2H₂O 20 μM; K₂HPO₄ 5.7 mM) containing 2% glucose as the main carbon source, and cultured at 45° C. and 250 rpm overnight. Seed from log phase of growth was then inoculated into 25 ml of the same fresh medium at an initial optical density at 600 nm (OD600) of 0.4. This second seed culture was cultured at 45° C. and 250 rpm overnight. The cultures for beta-carotene production were performed at 45° C. and 250 rpm for 24 h from log phase of growth inoculated into 25 ml of mineral define medium (NH₄)₂SO₄<100 mM; NaH₂PO₄.H₂O<10 mM; KCl<10 mM; Na₂SO₄<10 mM; Acide citrique<30 mM; MgCl₂.6H₂O<10 mM; CaCl₂.2H₂O<10 mM; ZnCl₂<50 mg/L; FeSO₄.7H₂O<50 mg/L; MnCl₂.4H₂O<50 mg/L; CuSO₄<50 mg/L; CoCl₂.6H₂O<50 mg/L; H₃BO₃<5 mg/L; MES<200 mM; (NH₄)₆Mo₇O₂₄.4H₂O<0.5 mM; Glucose<30 g/L (166 mM) at an initial optical density at 600 nm (OD600) of 0.4. After 24 h of culture, 1 mL of culture was centrifuged and carotenoid extraction was done by mixing 1 mL of ethanol with pellet. The ethanol phase was analyzed by absorbance at OD 455 nm and analyzed by HPLC (Column c18 poroshell agilent 150 mm*2.1 mm*2.7 um, mobile phase acetonitrile/methanol/ethyl acetate).

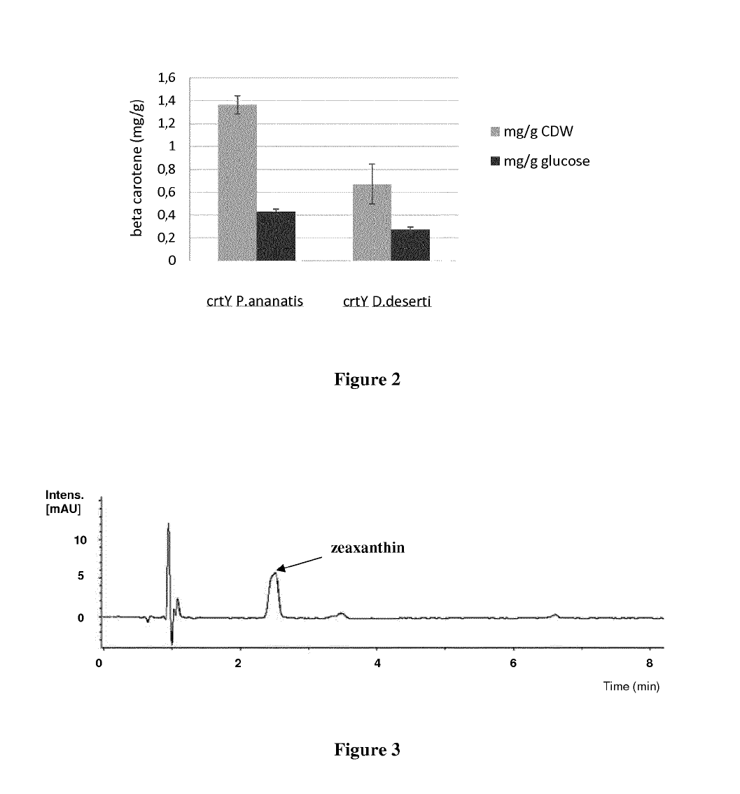

As shown in FIG. 2, beta-carotene production was obtained in strains B and C whereas no beta-carotene could be detected in the wild-type D. geothermalis strain or strain A.

### Example 3: Production of Zeaxanthin

A Deinococcus geothermalis strain was genetically engineered to produce zeaxanthin. This recombinant Deinococcus geothermalis strain was obtained by further modifying strain B of example 2. Indeed, the gene encoding the beta-carotene hydroxylase (CrtZ) from Pantoea agglomerans (SEQ ID NO: 13) was inserted into the chromosome replacing transposase (IS200/IS605) gene (DGEO_RS14195, (old locus tag: Dgeo_2273) NCBI Genbank: NC_008025.1) and was placed under the control of a constitutive promoter. The resulting constructs were checked by sequencing. This strain was named strain D.

To make seed cultures, individual colonies were picked to inoculate 25 ml of CMG2% medium (Peptone 2 g/L; Yeast Extract 5 g/L; Glucose 55 mM (20 g/L); MOPS acid 40 mM; NH₄Cl 20 mM; NaOH 10 mM; KOH 10 mM; CaCl₂.2H₂O 0.5 μM; Na₂SO₄.10H₂O 0.276 mM; MgCl₂.6H₂O 0.528 mM; (NH₄)₆(Mo₇)O₂₄.4H₂O 3 nM; H₃BO₃ 0.4 μM; CoCl₂.6H₂O 30 nM; CuSO₄.5H₂O 10 nM; MnCl₂ 0.25 μM; ZnSO₄.7H₂O 10 nM; D-Biotin 1 μg/L; Niacin (nicotinic acid) 1 μg/L; B6 vitamin 1 g/L; BI vitamin; FeCl₃ 20 μM; Sodium Citrate.2H₂O 20 μM; K₂HPO₄ 5.7 mM) containing 2% glucose as the main carbon source, and cultured at 45° C., 37° C. or 30° C. and 250 rpm overnight. Seed from log phase of growth was then inoculated into 25 ml of the same fresh medium at an initial optical density at 600 nm (OD600) of 0.4. This second seed culture was cultured at 45° C., 37° C. or 30° C. and 250 rpm overnight. The cultures for zeaxanthin production were performed at 45° C., 37° C. or 30° C. and 250 rpm for 24 h from log phase of growth inoculated into 25 ml of mineral define medium (NH₄)₂SO₄<100 mM; NaH₂PO₄.H₂O<10 mM; KCl<10 mM; Na₂SO₄<10 mM; Acide citrique<30 mM; MgCl₂.6H₂O<10 mM; CaCl₂.2H₂O<10 mM; ZnCl₂<50 mg/L; FeSO₄.7H₂O<50 mg/L; MnCl₂.4H₂O<50 mg/L; CuSO₄<50 mg/L; CoCl₂.6H₂O<50 mg/L; H₃BO₃<5 mg/L; MES<200 mM; (NH₄)₆Mo₇O₂₄.4H₂O<0.5 mM; Glucose<30 g/L (166 mM) at an initial optical density at 600 nm (OD600) of 0.4. After 24 h of culture 1 mL of culture was centrifuged and carotenoid extraction was done by mixing 1 mL of ethanol with pellet. The ethanol phase was analyzed by HPLC (Column c18 poroshell agilent 150 mm*2.1 mm*2.7 um, mobile phase acetonitrile/methanol/ethyl acetate).

As shown in FIG. 3, strain D expressing CrtZ from Pantoea agglomerans was able to produce zeaxanthin, contrary to the wild-type D. geothermalis strain or strains A to C. Furthermore, zeaxanthin was produced not only at 37° C. but also at 45° C. and 30° C. (data not shown).

### Example 4: Production of Canthaxanthin

A Deinococcus geothermalis strain was genetically engineered to produce canthaxanthin. This recombinant Deinococcus geothermalis strain was obtained by further modifying strain B of example 2. Indeed, the gene encoding the carotenoid 4,4′-beta-ionone ring oxygenase (CrtW) from Brevundimonas sp. SD212 (SEQ ID NO: 27) or from Paracoccus sp. N81106 (Agrobacterium aurantiacum) (SEQ ID NO: 31), or the beta-carotene ketolase (CrtO) from D. geothermalis (SEQ ID NO: 47) was inserted into chromosome, replacing transposase (IS200/IS605) genes, and was placed under the control of a constitutive promoter. The resulting constructs were checked by sequencing. The strain expressing CrtW from Brevundimonas sp. SD212 was named strain E, the strain expressing CrtW from Paracoccus sp. N81106 was named strain F and the strain expressing CrtO from D. geothermalis was named strain G.

To make seed cultures, individual colonies were picked to inoculate 25 ml of CMG2% medium (Peptone 2 g/L; Yeast Extract 5 g/L; Glucose 55 mM (20 g/L); MOPS acid 40 mM; NH₄Cl 20 mM; NaOH 10 mM; KOH 10 mM; CaCl₂.2H₂O 0.5 μM; Na₂SO₄.10H₂O 0.276 mM; MgCl₂.6H₂O 0.528 mM; (NH₄)₆(Mo₇)O₂₄.4H₂O 3 nM; H₃BO₃ 0.4 μM; CoCl₂.6H₂O 30 nM; CuSO₄.5H₂O 10 nM; MnCl₂ 0.25 μM; ZnSO₄.7H₂O 10 nM; D-Biotin 1 μg/L; Niacin (nicotinic acid) 1 μg/L; B6 vitamin 1 g/L; BI vitamin; FeCl₃ 20 μM; Sodium Citrate.2H₂O 20 μM; K₂HPO₄ 5.7 mM) containing 2% glucose as the main carbon source, and cultured at 45° C. (strain G) or 30° C. (strains E and F) and 250 rpm overnight. Seed from log phase of growth was then inoculated into 25 ml of the same fresh medium at an initial optical density at 600 nm (OD600) of 0.4. This second seed culture was cultured at 45° C. (strain G) or 30° C. (strains E and F) and 250 rpm overnight. The cultures for canthaxanthin production were performed at 45° C. (strain G) or 30° C. (strains E and F) and 250 rpm for 24 h from log phase of growth inoculated into 25 ml of mineral define medium (NH₄)₂SO₄<100 mM; NaH₂PO₄.H₂O<10 mM; KCl<10 mM; Na₂SO₄<10 mM; Acide citrique<30 mM; MgCl₂.6H₂O<10 mM; CaCl₂.2H₂O<10 mM; ZnCl₂<50 mg/L; FeSO₄.7H₂O<50 mg/L; MnCl₂.4H₂O<50 mg/L; CuSO₄<50 mg/L; CoCl₂.6H₂O<50 mg/L; H₃BO₃<5 mg/L; MES<200 mM; (NH₄)₆Mo₇O₂₄.4H₂O<0.5 mM; Glucose<30 g/L (166 mM) at an initial optical density at 600 nm (OD600) of 0.4. After 24 h of culture 1 mL of culture was centrifuged and carotenoid extraction was done by mixing 1 mL of ethanol with pellet. The ethanol phase was analyzed by HPLC (Column c18 poroshell agilent 150 mm*2.1 mm*2.7 um, mobile phase acetonitrile/methanol/ethyl acetate).

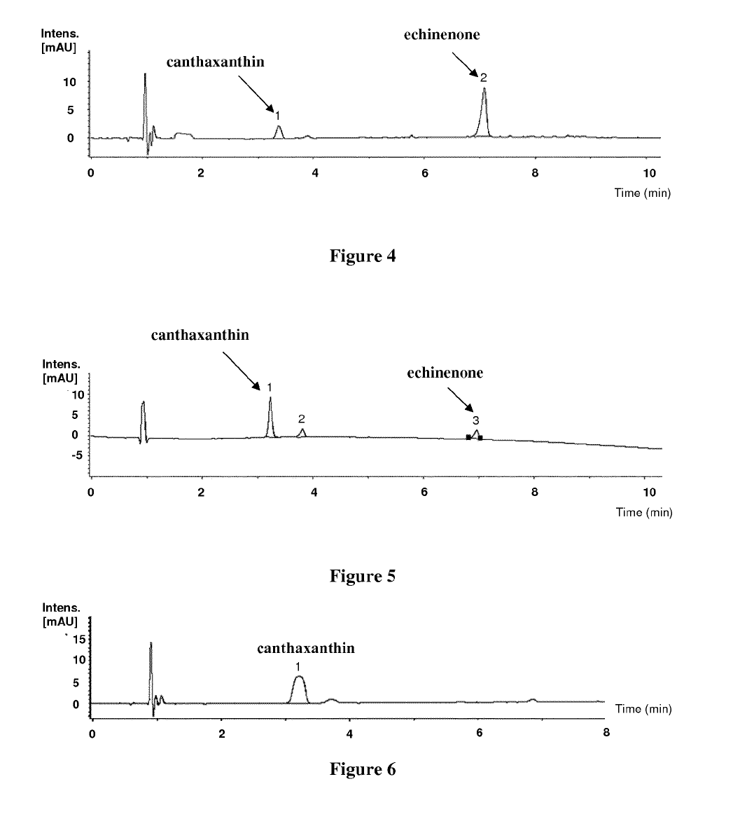

As shown in FIGS. 4 to 6, strains E, F and G expressing CrtW from Brevundimonas sp. SD212, CrtW from Paracoccus sp. N81106 and CrtO from D. geothermalis, respectively, were able to produce canthaxanthin, contrary to the wild-type D. geothermalis strain or strains A to D.

### Example 5: Production of Astaxanthin

A Deinococcus geothermalis strain was genetically engineered to produce astaxanthin. This recombinant Deinococcus geothermalis strain was obtained by further modifying strain D of example 3 producing zeaxanthin. Indeed, the gene encoding CrtO from D. geothermalis (SEQ ID NO: 47) or encoding CrtW from Paracoccus sp. N81106 (SEQ ID NO: 31), was inserted into the chromosome, replacing the transposase (IS200/IS605) gene or replacing the phosphotransacetylase (pta) gene (DGEO_RS02840 NCBI Genbank: NC_008025.1), and was placed under the control of a constitutive promoter. The resulting constructs were checked by sequencing. The strain expressing CrtY from P. ananatis, CrtZ from P. agglomerans and CrtO from D. geothermalis, was named strain H. The strain expressing CrtY from P. ananatis, CrtZ from P. agglomerans and CrtW from Paracoccus sp. N81106, was named strain I.

To make seed cultures, individual colonies were picked to inoculate 25 ml of CMG2% medium (Peptone 2 g/L; Yeast Extract 5 g/L; Glucose 55 mM (20 g/L); MOPS acid 40 mM; NH₄Cl 20 mM; NaOH 10 mM; KOH 10 mM; CaCl₂.2H₂O 0.5 μM; Na₂SO₄.10H₂O 0.276 mM; MgCl₂.6H₂O 0.528 mM; (NH₄)₆(Mo₇)O₂₄.4H₂O 3 nM; H₃BO₃ 0.4 μM; CoCl₂.6H₂O 30 nM; CuSO₄.5H₂O 10 nM; MnCl₂ 0.25 μM; ZnSO₄.7H₂O 10 nM; D-Biotin 1 μg/L; Niacin (nicotinic acid) 1 μg/L; B6 vitamin 1 μg/L; B1 vitamin; FeCl₃ 20 μM; Sodium Citrate.2H₂O 20 μM; K₂HPO₄ 5.7 mM) containing 2% glucose as the main carbon source, and cultured at 37° C. (strain H) or 30° C. (strain I) and 250 rpm overnight. Seed from log phase of growth was then inoculated into 25 ml of the same fresh medium at an initial optical density at 600 nm (OD600) of 0.4. This second seed culture was cultured at 37° C. (strain H) or 30° C. (strain I) and 250 rpm overnight. The cultures for astaxanthin production were performed at 37° C. (strain H) or 30° C. (strain I) and 250 rpm for 24 h from log phase of growth inoculated into 25 ml of mineral define medium (NH₄)₂SO₄<100 mM; NaH₂PO₄.H₂O<10 mM; KCl<10 mM; Na₂SO₄<10 mM; Acide citrique<30 mM; MgCl₂.6H₂O<10 mM; CaCl₂.2H₂O<10 mM; ZnCl₂<50 mg/L; FeSO₄.7H₂O<50 mg/L; MnCl₂.4H₂O<50 mg/L; CuSO₄<50 mg/L; CoCl₂.6H₂O<50 mg/L; H₃BO₃<5 mg/L; MES<200 mM; (NH₄)₆Mo₇O₂₄.4H₂O<0.5 mM; Glucose<30 g/L (166 mM) at an initial optical density at 600 nm (OD600) of 0.4. After 24 h of culture 1 mL of culture was centrifuged and carotenoid extraction was done by mixing 1 mL of methanol with pellet. The methanol phase was analyzed by HPLC (Column c18 poroshell agilent 150 mm*4.6 mm*2.7 um, mobile phase (85% MeOH, 5.5% acetonitrile, 5% dichloromethane, 4.5% H₂O)).

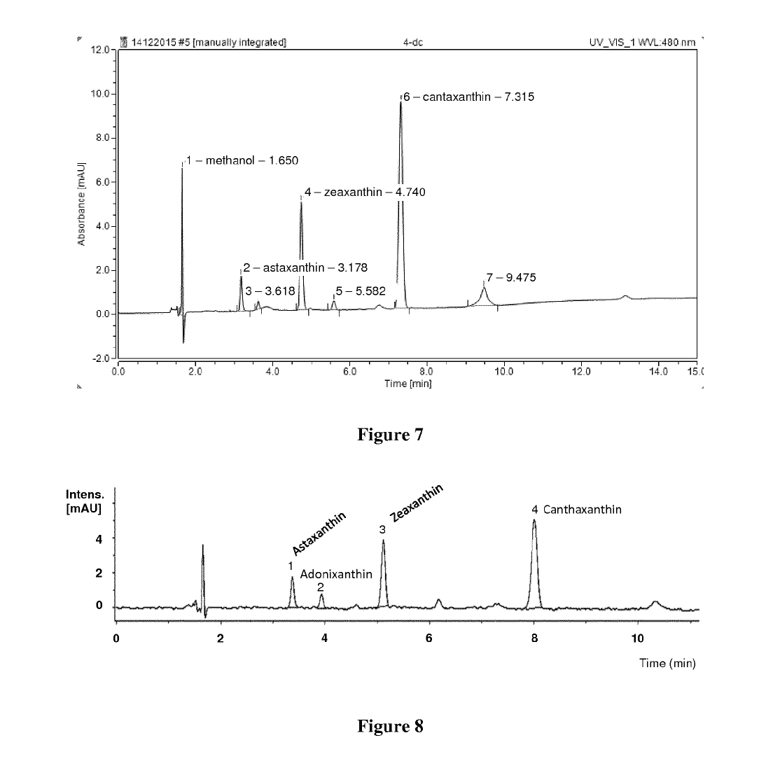

As shown in FIGS. 7 and 8, strain H expressing CrtY from P. ananatis, CrtZ from P. agglomerans and CrtO from D. geothermalis and strain I expressing CrtY from P. ananatis, CrtZ from P. agglomerans and CrtW from Paracoccus sp. N81106, were able to produce astaxanthin, contrary to the wild-type D. geothermalis strain or strains A to G.

### Example 6: Production of Beta-Carotene

Several Deinococcus geothermalis bacteria were genetically engineered to produce beta-carotene as described above in example 2. Briefly, the gene encoding the lycopene beta-cyclase (CrtY) from Pantoea agglomerans (SEQ ID NO: 9), Porphyrobacter cryptus (SEQ ID NO: 75), Franconibacter pulveris (SEQ ID NO: 77), Phormidium tenue (SEQ ID NO: 79), Siccibacter colletis (SEQ ID NO: 81), Erythrobacter vulgaris (SEQ ID NO: 83), Rhizobium sp. Leaf321 (SEQ ID NO: 85) or Parvularcula oceani (SEQ ID NO: 87) was inserted into the chromosome and placed under the control of a constitutive promoter. The resulting constructs were checked by sequencing. The cultures for beta-carotene production were performed at 48° C. and 250 rpm for 24 h.

As shown in Table 1 below, each of these recombinant bacteria was able to produce beta-carotene at 48° C.

TABLE 1
Production of beta-carotene

| Origin of the CrtY gene | Production of beta-carotene |
|---|---|
| P. ananatis | + |
| P. agglomerans | +++ |
| Porphyrobacter cryptus | +++ |
| Franconibacter pulveris | +++ |
| Phormidium tenue | ++ |
| Siccibacter colletis | + |
| Erythrobacter vulgaris | ++ |
| Rhizobium sp. | ++ |
| Parvularcula oceani | ++++ |

+: detection of beta-carotene,
++: good production of beta-carotene,
+++: high level of beta-carotene production,
++++: very high level of beta-carotene production.

### Example 7: Production of Zeaxanthin

Several Deinococcus geothermalis bacteria were genetically engineered to produce zeaxanthin as described above in example 3. Briefly, the gene encoding the beta-carotene hydroxylase (CrtZ) from Franconibacter pulveris (SEQ ID NO: 89), Siccibacter colletis (SEQ ID NO: 91) or Cronobacter malonaticus (SEQ ID NO: 93) was inserted into the chromosome of strain B of example 2 and placed under the control of a constitutive promoter. The resulting constructs were checked by sequencing. The cultures for zeaxanthin production were performed at 42° C. and 250 rpm for 24 h.

The inventors found that each of these recombinant bacteria was able to produce zeaxanthin and cryptoxanthin when cultured at 42° C.

1-33. (canceled)

34. A recombinant Deinococcus bacterium comprising a heterologous nucleic acid sequence encoding a polypeptide exhibiting lycopene cyclase activity and being able to produce beta-carotene when cultured at a temperature greater than 40° C.

35. The recombinant bacterium of claim 34, further comprising a heterologous nucleic acid sequence encoding a polypeptide exhibiting beta-carotene hydroxylase activity.

36. The recombinant bacterium of claim 34, further comprising a nucleic acid sequence encoding a polypeptide exhibiting beta-carotene ketolase activity.

37. The recombinant bacterium of claim 34, wherein the polypeptide exhibiting lycopene cyclase activity is selected from the group consisting of SEQ ID NOs: 1, 3, 5, 7, 9, 75, 77, 79, 81, 83, 85 and 87, and polypeptides exhibiting lycopene cyclase activity and having at least 80% identity to SEQ ID NO: 1, 3, 5, 7, 9, 75, 77, 79, 81, 83, 85 or 87.

38. The recombinant bacterium of claim 35, wherein the polypeptide exhibiting beta-carotene hydroxylase activity is selected from the group consisting of SEQ ID NOs: 11, 13, 15, 17, 19, 21, 23, 25, 89, 91 and 93 and polypeptides exhibiting beta-carotene hydroxylase activity and having at least 80% identity to SEQ ID NO: 11, 13, 15, 17, 19, 21, 23, 25, 89, 91 or 93.

39. The recombinant bacterium of claim 36, wherein the polypeptide exhibiting beta-carotene ketolase activity is selected from the group consisting of beta-carotene ketolase of SEQ ID NOs: 27, 29, 31, 33, 35, 37, 39, 41, 43, 45, 47, 67, 69, 71, 73, 95 and 97, and polypeptides exhibiting beta-carotene ketolase activity and having at least 80% identity to SEQ ID NO: 27, 29, 31, 33, 35, 37, 39, 41, 43, 45, 47, 67, 69, 71, 73, 95 or 97.

40. The recombinant bacterium of claim 34, wherein the endogenous gene encoding LmbE like protein, the gene encoding carotenoid 3′,4′-desaturase (CrtD), the gene encoding glucosyltransferase (CruC), the gene encoding acyltransferase (CruD) and/or the gene encoding C-1′,2′ hydratase (CruF), are inactivated.

41. The recombinant bacterium of claim 34, wherein the endogenous gene encoding endogenous CrtO-type ketolase is inactivated.

42. The recombinant bacterium of claim 34, wherein the bacterium further exhibits increased FPP synthase, DXP synthase, IPP isomerase, phytoene synthase and/or phytoene desaturase activities by comparison to the wild-type bacterium.

43. The recombinant bacterium of claim 34, wherein said bacterium is a thermophilic Deinococcus.

44. The recombinant bacterium of claim 34, wherein said recombinant bacterium produces astaxanthin, or a precursor thereof, when cultured at a temperature between 42 and 48° C.

45. The recombinant bacterium of claim 44, wherein said recombinant bacterium produces zeaxanthin when cultured at a temperature between 42 and 48° C.

46. The recombinant bacterium of claim 44, wherein said recombinant bacterium produces canthaxantin when cultured at a temperature between 42 and 48° C.

47. A method of producing a compound selected from astaxanthin and a precursor thereof, comprising culturing a recombinant Deinococcus bacterium according to claim 34 under conditions suitable to produce said compound, and optionally recovering said compound.

48. The method of claim 47, wherein the compound is selected from the group consisting of β-carotene, echinenone, canthaxantin, adonirubin, 3′hydroxyechinenone, 3-hydroxyechinenone, β-cryptoxanthin, zeaxanthin and adonixanthin.

49. The method of claim 48, wherein the compound is β-carotene.

50. The method of claim 48, wherein the compound is canthaxantin.

51. The method of claim 48, wherein the compound is zeaxanthin.

52. The method of claim 48, wherein the recombinant Deinococcus bacterium is cultured at a temperature between 37 and 50° C.


SEQUENCE LISTING (98 sequences)

SEQ ID NO: 1 - protein, 382 aa, Pantoea ananatis
MQPHYDLILVGAGLANGLIALRLQQQQPDMRILLIDAAPQAGGNHTWSFHHDDLTESQHRWIAPLVVHHWPDYQVRFPTRRRKLNSGYFCITSQRFAEVLQRQFGPHLWMDTAVAEVNAESVRLKKGQVIGARAVIDGRGYAANSALSVGFQAFIGQEWRLSHPHGLSSPIIMDATVDQQNGYRFVYSLPLSPTRLLIEDTHYIDNATLDPECARQNICDYAAQQGWQLQTLLREEQGALPITLSGNADAFWQQRPLACSGLRAGLFHPTTGYSLPLAVAVADRLSALDVFTSASIHHAITHFARERWQQQGFFRMLNRMLFLAGPADSRWRVMQRFYGLPEDLIARFYAGKLTLTDRLRILSGKPPVPVLAALQAIMTTHR

SEQ ID NO: 2 - DNA, 1149 bp, Pantoea ananatis
atgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaa

SEQ ID NO: 3 - protein, 562 aa, Marchantia polymorpha
MASCTVSMSSTRYGLRAGAVKVRSGSPKHLLRSGFCPIAPTRGDELSSRCGEVNGNGSLLSPGLAARRLHMAETRVCSVRAAGGTLLEMKPKVELKKLNYEIPIFDPRKSKAVDLVVVGGGPAGLAVAQRVSGQGLSVCCIDPEPRSVWPNNYGVWVDEFEAMDLLDCLDYTWGSAVVYLDDDVKKLLQRPYGRVNRTRLKSKMIEQCITNGVQFYQSKVKHVTHSKDRSFVVCDDGFVIETAVVLDATGHSRRLVQYDKPFNPGYQIAYGIVAEVDCHPFDLDKMLFMDWRDSHLNKYPELKESNKKLPTFLYAMPFSPTKIFLEETSLVARPGVPLEVIQQRMEARLKHLGINVKSIEEDEVCVIPMGGVLPVIPQRVLGIGGTAGMVHPSTGYMVARTLAAAPIVADSLVKRLGGASHPHISSNTVLGKEAEPSSSQDEDIPTPPEPRKGPFGDEVAAGVWADLWPMERQQQREFFCFGMDVLLKLDLVGTRRFFDAFFDLDEHHWQGFLSSRLYFWELIGFGFSLFTHATNTAKAEIVVKGTPALLNMIKNLTLMKRQ

SEQ ID NO: 4 - DNA, 1689 bp, Marchantia polymorpha
atggcgtcgtgcacggtgtcgatgagctcgacgaggtatgggttgagagcaggggcggtgaaagtgaggagcggctcgccgaagcacctgttgcgttccggcttctgtccgattgcgccgacgcgaggggacgaattgagctctcgatgtggggaagtgaatgggaatgggagtttgctgtctcccggactggcggctcggcgtctgcacatggcggagacgagagtttgctctgtgagggccgcaggtggtactctgctcgagatgaagcccaaagtcgagctgaagaaactgaactatgagattccaatttttgatccgaggaagagtaaggctgtggatctcgtggtcgtcgggggaggtcctgcaggtttagccgtagcccagagagtgtcaggtcaaggactgagcgtttgctgcatcgacccggagccgaggagtgtgtggcccaacaattatggagtttgggtggacgaattcgaggcaatggacctgctggactgtctcgactacacgtggggaagcgccgtggtttacttggatgatgacgtaaagaagttactgcaacgtccttacggaagagtgaatcgaaccaggttgaagtcgaagatgattgagcagtgcataacgaatggggtgcagttttaccagtcgaaggtgaaacacgttacccacagcaaggaccgatcttttgtggtctgcgatgatggattcgtgattgagactgcagttgttttggatgccacagggcactccagacgtcttgtgcagtatgacaagcctttcaatccaggctatcaaattgcatacggtatagttgccgaggttgactgccatccttttgaccttgacaaaatgctgttcatggactggagggactcccatttgaacaaatatcctgaactcaaagaaagcaacaagaagctgccaactttcttgtatgccatgccattttctccgacgaaaatatttttggaggaaacatctctcgttgcaaggcccggagtgcccctggaggtgatccagcaacggatggaggcacggttgaaacatctgggtatcaatgtgaagagcattgaagaggacgaagtgtgtgtcatcccaatgggaggagtgctccctgtgattcctcaaagggtcttggggatcggaggaacggcagggatggttcatccttcaactggttacatggtggcgcgaactctggctgccgcaccgatcgttgcggactctttggtgaaaaggcttggtggcgcaagccatccccacatatcttccaacactgtcctggggaaggaagcagagccatcttctagtcaggatgaggacattccgactcctccagaaccccggaaaggaccctttggagatgaagtagcagcaggagtatgggctgacttatggcctatggaacgacaacagcaaagggagtttttctgttttggaatggacgtactcctaaagcttgaccttgttggaacgaggcgattctttgatgcatttttcgaccttgacgagcatcattggcaagggttcttgtcttctagattgtacttctgggagcttattgggtttggattttcgctcttcacgcatgcgaccaacactgcaaaggccgagattgtggtcaagggaactccagctcttttgaacatgatcaagaacttaacattgatgaagcggcaataa

SEQ ID NO: 5 - protein, 577 aa, Haematococcus pluvialis
RTFLSQTAIMLSPLQRLCAVPGSSYSAGTALVLLPPAPRVCLSRQHEQTTQWPRRRLVRASAATASEQRPARSSPSGYIMRDPMPWPTASDIALTQHDLQATPEVDLIVAGAGPSGIAVAERVAAAGFSVCVVDPEPLGIWPNNYGAWVDEFEAMGLGEYMEIVWPKASVHLSNKPEGEKFLSRPYGRVDRPRLKSMLLKKCAAHGVTFLEGKVDGVNHGEGRSAVSLADGRSIRGSLVLDATGHVRKLIKFDQKFDPGYQGAYGIVAEVESHPFDLETMLFMDWRDEHTQGNPAMHAANNALPTFLYAMPFSKTRIFLEETSLVARPAVGFDELKERLDARLKWLGIKVKAVEEEEYCLIPMGGVLPTHPQRVLGIGGTAGMVHPSTGFMMSRMLGAVPTIADAIVDQLSAPADKATSLAARKPGSEAEAEAMSAAVWRAAWPVERIRQRLFNTFGMELLLSLDLQQTRDFFSAFFNLSDFHWHGFLSTRLSFSQLLGFGITLFFKSSNNIRIHLLKLGVPGLVRILFMLAPTLRGYYKHAPTVRQQKAVADAAAAVAARTAAAAKPADLPPAMIK

SEQ ID NO: 6 - DNA, 1735 bp, Haematococcus pluvialis
gcgcacgttcctgtcacaaacggcaatcatgttgtcgccgctgcagagattgtgtgcagtgcccgggagcagctactctgcagggaccgcgctagtgctgcttccccccgcgcctcgcgtgtgtttgagcaggcaacacgaacagaccactcaatggccacgacgacgcttagtgcgggcttcggctgccacagccagcgagcagcgaccagcgcgtagctcaccgtcaggctatattatgcgcgatcctatgccttggcccaccgctagcgacattgctttgacccagcacgacttgcaagccacgcccgaggtggacctcatcgtggctggtgccgggccatctggcatagctgtagcagagcgggtcgctgcggccgggttctcggtgtgcgttgttgacccagagccgttgggcatttggcccaacaactacggggcatgggtggatgagttcgaggccatgggcctgggggagtacatggagattgtgtggcccaaggcctcggtgcatctcagcaacaagccagagggcgagaagttcctaagtcgcccctacggtcgcgtggaccggccacgcttgaaaagcatgctgctgaagaagtgtgcggcccatggagtgacctttctggaaggcaaagttgacggggtcaaccatggtgagggacgctccgccgtcagcctggctgatgggcgcagcatccgcggctctctggtgctggacgccactggccacgtgcgcaagctgatcaagttcgaccagaagtttgacccaggttatcagggcgcgtacggcattgtggcagaggtggagtctcacccattcgacctggagaccatgctgttcatggactggcgtgatgagcacactcagggcaaccccgcaatgcatgcggccaacaacgccctgcctacctttttgtacgccatgcccttctccaagaccaggatattcctggaagagacgtccctggtggcgcggccagctgtcgggtttgacgagctgaaggagcgcctggacgcacgtctcaagtggctgggcatcaaggtcaaggcagtggaggaggaggaatactgcctgatccctatgggaggggtgctgcctacccacccccaacgtgtgctgggcatcggcggcacggcgggcatggtgcatccatcaacagggttcatgatgtcacgcatgttgggcgccgtacccaccatcgctgacgccatcgtggaccaactgtccgcccctgctgacaaggccacaagtttagctgcccgcaagcctggcagcgaggccgaagctgaagccatgtctgctgcggtgtggcgcgccgcctggcccgtggagcgcatccgccagcgcttgttcaacacctttggcatggagctgctgctgtccctggacctgcagcagacacgcgacttcttctccgccttcttcaacttgtcagacttccactggcatggcttcctgtccactcggctgtccttctcccagctgcttggcttcggcatcacgctcttcttcaagtcttccaacaacatccgcatccacctgctcaagctgggcgtgccaggcctcgtccgcatcctcttcatgttggcgcccacgcttcgcggctactacaagcacgcgcccactgtgcggcagcagaaggccgtagcggacgccgccgctgctgtagcggcgcggacggccgcagcggccaagccagcagatctgccgccagccatgatcaagtag

SEQ ID NO: 7 - protein, 482 aa, Deinococcus deserti
MTYLQYHLVFIVPVLLVLAALTARRRGPVAGRYNTNDRWSWSWFWALPLVAAAYTTPWDNYLVFREVWSYPPDRVLGRLGYVPYEEYAFFFLQTLITGLLLLLLLRRGGPARIASRPELVRWGGAALLLGLAFIGALCLRQESTLYLGLILAWAMPVLAGQWAFGGDLLMGRARLFWTATLLPTVYLWLTDAYAITHGIWQISARYTTGLNLGPLPLEEMLFFLVTNLLVVTGLMLFLHPAALERLGRARPYLRPWLGFAALYLLAKIPVPLWPGGFPLLGMLSTVFLCLAALTYAAQRVGWGRALSLLLLGSGTGWLVEWVGSQTGLPFGHYSYQGAPAPTLLGVPLIVPLGWFAMTLVATLLSGGRAWLAGLLLVAWDLGLEPLMTSQGYWTWQDPHPLWSGAPLLNFVGWWGIGTALSWAFVRLAPGLQPAASALHPGQVYLVELLFLPGGLLLFGQPVAAGVTLVAMALVLSQTRRAV

SEQ ID NO: 8 - DNA, 1449 bp, Deinococcus deserti
atgacgtacctgcagtaccacctggtgtttatcgtgccggtgctgctggtgctggcggcactgaccgcgcggcgccgtggtccagtggcaggccggtataacaccaacgaccgctggagctggagctggttctgggccctgccgctggtggccgccgcatacaccactccctgggacaactatctggtgttccgcgaggtgtggtcctacccgcccgaccgggtgctgggccgtctgggatacgtgccttacgaggagtacgcgttctttttcctccagaccctgatcacgggtctgctgctgctgctgctgctgcgccggggcggaccggcacggatcgcgtcccggccagagctcgtccggtggggcggcgcggcgctgctgctggggcttgcctttataggagcgctgtgtctgcgtcaggaaagcacgctgtacctgggtctgatcctggcctgggccatgcccgtcctggccggacagtgggcctttgggggagacctgctgatgggccgcgcacggctgttctggaccgcgacgctgctgcccacggtgtacctgtggctgaccgacgcgtacgcgatcacgcacggcatctggcagatttccgcgcggtacaccactggcctgaacctgggcccccttcctcttgaggagatgctgtttttcctggtgaccaacttgctggtggtcactggactgatgctgttcctgcatccggcggctcttgagcgccttggccgcgcccgtccgtacctgcgtccatggctgggctttgcagcgctgtacctgctcgccaaaattccggttcccctgtggccaggtgggttcccgttgctcggaatgctgagcacggtgtttctgtgcctggcagctctgacatacgccgcccagcgggtgggttggggccgggccctgtctctgctgctgctgggcagcgggacgggctggctggttgagtgggtcggcagccagaccgggcttcctttcggccactacagctaccagggcgctccggcccccacgcttctgggcgttcccctgattgtgccgctgggctggtttgccatgaccctggtggccacgctgctgtcgggtgggcgggcgtggctcgcgggactgctgctggtggcctgggacctgggacttgaacccctgatgacctcccagggctactggacctggcaggacccccacccgctgtggagcggcgctccactgctgaatttcgtcggctggtggggcataggcaccgcgctgagctgggcgttcgtgcggctggccccgggtttgcagccggcagcttcggccctccatccggggcaggtgtatctggtcgaactgctgtttctgcccggaggtctgctgctgttcggtcagccggtcgctgcaggcgtcaccctggtggccatggcgctggtcctgagccagacccggcgggcggtgtga

SEQ ID NO: 9 - protein, 386 aa, Pantoea agglomerans
MPRYDLILVGAGLANGLIALRLRQQRPSLRILLIDAEREPGANHTWSFHAEDLTETQHRWIAPLVVHHWPGYEVRFPQRSRSLNSGYFCVTSERFVQVIRDRFAPDLLLNTRVAGIASRTVTLDDGRVLESDAVIDGRGYQPDAALCMGFQSFVGQEWQLSEPHGLTAPIIMDATVDQQAGYRFVYSLPFSADTLLIEDTHYIDNATLEGDRARQNIRAYAAQQGWRLDRLLREEQGALPITLTGDVAAFWQKHDLPCSGLRAGLFHPTTGYSLPLAVALADRLAQMQTFTSETLHATIQQFASQAWQQQRFFRMLNRMLFLAGPADQRWQVMQRFYGLPEGLIARFYAGKLTLPDRLRILSGKPPVPVLAALQAIMTPHRQQAMQ

SEQ ID NO: 10 - DNA, 1161 bp, Pantoea agglomerans
atgccgcggtatgatctgattctggtgggcgcgggactggccaacggtctgatcgccctgcgcctgcggcagcagcggccttccctgcgcattctgctgattgacgccgaacgtgaaccgggtgccaatcacacctggtcgtttcatgcggaagatctcaccgaaacgcagcatcgctggatcgctccgctggtagtacatcactggcctggctatgaggtccgctttccccaacgcagtcgcagtctgaacagtggctatttttgcgtgactgcggagcgcttcgcgcaggtcatccgcgacaggtttgcgccggatctgctgctgaatacccgggttgcgggcatcgcttcacgcacggttacgctggacgatggccgggtgctggagagtgacgcagtgattgatggccgtggctaccagcccgatgccgcgctgcgcatgggcttccagtcgtttgtcggacaggagtggcagctcagcgagccacatggcctgaccgcgccgatcatcatggatgccacggtagatcagcaggcaggctatcgctttgtctacagcctgcccttttctgcagacaccctgctgattgaagatacccattacattgataacgccacgctggaaggcgatcgcgcccgtcagaatatccgcgactatgccgcgcagcaggggtggcgattagaccggctgctacgcgaagagcaaggcgcattacccattacgctgaccggcgacgtggtcgccttctggcagaaacgcgagctgccctgcagcggtttgcgtgccgggttattccatccgactaccggctattctctgccgctggcggtcgcgctggctgaccggctggcgcagatgcagacgtttacctctgagaccctgcacgccaccattcagcaattcgccagtcaggcctggcagcaacagcgcttcttccgcatgctgaaccgtatgctgtttctggcgggtccggctgaccagcgctggcaggttatgcaacgtttttacggccttcccgaaggtctgatcgcccgtttttatgcgggaaaactcactttgcctgatcggctgcgcatcttaagcggcaagcccccggtccccgttctggcggcgttacaggctattatgactcctcaccgtcaacaggcgatgcaatga

SEQ ID NO: 11 - protein, 340 aa, Marchantia polymorpha
MLKVVASGATAVASLGVVRSGRECGRDGIGLEQLRHRALPSFPSLGLSSLEFNPLMTRTGIQRRIRIQRSIGPPSVLQIDEHQHGESPAPIEEHLLETEQSADVADKVESSFPDTPAVSKMQQRLTKRQTERKAYLLAAIASTTGFTTLAVAAVFYRFIWQMQGSGEIPYTEIFGTFALAVGAAVGMEYWARWAHKALWHASLWNMHESHHRPREGPFEMNDIFAIINAVPAVSLMLYGFLNRGLVPGLCFGAGLGITMFGIAYMFVHDGLVHRRFPVGPIADVPYLQKVAAAHQLHHADLYEGVPYGLFLGPKELEEVGGLDELERVMKQRAKPSASSK

SEQ ID NO: 12 - DNA, 1023 bp, Marchantia polymorpha
atgctgaaggtcgtagcatctggcgccaccgctgttgcctctctcggggtcgtgaggagcggtcgtgaatgtgggcgagatgggattgggctcgagcagctgagacacagggcgctgccgagcttcccgagtctgggattgagcagcttggaatttaatccgttgatgacgagaaccggaattcaacgcaggattcgaattcagcgcagcatcggtcctccctccgttttgcagattgatgagcaccagcatggtgaatctccagctcccatcgaggagcacctgctcgagactgagcaatccgctgatgttgccgacaaagttgagagcagttttcccgacactcccgctgtcagtaaaatgcagcaaagattgaccaagagacaaacagaacgtaaagcatacctcttagcagccatcgcatctacgacaggattcaccacgctcgccgtcgccgccgtcttctatcgttttatctggcaaatgcagggaagtggcgagatcccgtacacagaaatattcggaacatttgccctcgctgtcggagccgcggttgggatggaatactgggctagatgggcgcacaaagctctgtggcacgcatcgctgtggaacatgcacgagtcacatcaccgacccagagaaggaccttttgaaatgaacgacatttttgctatcataaacgcagttcccgccgtctctctgatgctctacggatttcttaacagaggacttgttcctggtctctgcttcggagcgggtttaggcatcactatgttcggtatagcctacatgttcgttcacgatggtcttgtacaccgacgattcccagtcggacctatcgccgatgtcccatatcttcagaaggttgccgcagcccatcagcttcatcacgctgacctttacgagggtgtaccctatggtcttttcctcggcccaaaggagctggaagaagttggaggattggacgaactcgagagagtcatgaagcagagagccaagccctctgcatcctccaagtag

SEQ ID NO: 13 - protein, 176 aa, Pantoea agglomerans
MLVNSLIVILSVIAMEGIAAFTHRYIMHGWGWRWHESHHTPRKGVFELNDLFAVVFAGVAIALIAVGTAGVWPLQWIGCGMTVYGLLYFLVHDGLVHQRWPFHWIPRRGYLKRLYVAHRLHHAVRGREGCVSFGFIYARKPADLQAILRERHGRPPKRDAAKDRPDAASPSSSSPE

SEQ ID NO: 14 - DNA, 531 bp, Pantoea agglomerans
atgctagtaaatagtttaatcgtcatcttgagcgttattgcgatggagggcatcgccgcgtttacccaccgctacattatgcacggctggggatggcgctggcatgagtcacaccataccccgcgcaagggcgtatttgagctaaacgatctctttgcggtggtgtttgccggggtggctatcgcgctgattgccgtgggcacggcgggcgtttggcccctgcagtggattggctgcggcatgacggtctatggcctgctctacttcctggtgcacgacggtctggtgcatcagcgctggcccttccactggatcccgcgccggggctacctgaagcggctctacgtcgcccaccgcctgcaccacgcggtgcgcggccgggagggctgcgtctccttcggttttatttacgcccgcaagcctgccgacctacaggcgatcctgcgtgaacgtcatggccgcccgcctaaacgggacgctgccaaagaccggccggacgcggcgtcaccctcgtcgtcttcgcccgaataa

SEQ ID NO: 15 - protein, 296 aa, Haematococcus pluvialis
ITTMLSKLQSISVKARRVELARDITRPKVCLHAQRCSLVRLRVAAPQTEEAVGTQQAAGAGDEHSADVALQQLDRAIAERRARRKREQLSYQAAAIAASIGVSGIAIFATYLRFAMHMTVGGAVPWGEVAGPLLLVVGGQLGMEMYARYAHKAIWHESPLGWLLHKSHHTPRTGPFEANDLFAIIKGLPAMLLCTFGFWLPNVLGTACFGAGLGITLYGMAYMFVHDGLVSRRFPTGPIAGLPYMKRLTVAHQLHHSGKYGGAPWGMFLGPQELQHIPGAAEEVERLVLELDWSKR

SEQ ID NO: 16 - DNA, 891 bp, Haematococcus pluvialis
attaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcggtgggaacccagcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggccctctcctcttggtggttggtggccagctcggcatggagatgtatgcccgctatgcacacaaagccatctggcatgagtcgcctctgggctggctgctgcacaagagccaccacacacctcgcactggaccctttgaagccaacgacttgtttgcaatcatcaaaggactgcccgccatgctcctgtgtacctttggcttctggctgcccaacgtcctggggacggcctgctttggcgcggggctgggcatcacgctatacggcatggcatatatgtttgtacacgatggcctggtttccaggcgctttcccaccgggcccatcgctggcctgccctacatgaagcgcctgacagtggcccaccagcttcaccacagcggcaagtacggtggcgcgccctggggcatgttcttgggtccacaggagctgcagcacataccaggtgcggcggaggaggtggagcgactggtcctggaactggactggtccaagcggtag

SEQ ID NO: 17 - protein, 161 aa, Brevundimonas sp. SD212
MAWLTWIALFLTAFLGMEAFAWIMHRYVMHGFLWSWHRSHHEPHDHPLEKNDLFAVVFAAPAIVMVAVGLHLWPWALPVGLGITAYGMVYFFFHDGLVHRRFPTGFSGRSGFWTRRIQAHRLHHAVRTREGCVSFGFLWVRSARALKAELAQKRGSSSSGA

SEQ ID NO: 18 - DNA, 486 bp, Brevundimonas sp. SD212
atggcatggctgacctggatcgcactgttcctgaccgcattcctgggtatggaggctttcgcgtggatcatgcaccgttatgtcatgcacggtttcttgtggtcgtggcatcgtagccatcacgagccgcacgaccacccgctggaaaagaacgacctgtttgccgttgtctttgccgctccggcgattgttatggtggcggtgggtctgcacctgtggccttgggccttgccggtcggtctgggtattactgcgtacggcatggtttacttcttctttcatgatggcctggtgcatcgtcgtttcccgacgggctttagcggtcgcagcggcttttggacccgtcgcatccaggcgcaccgtctgcaccatgcagtccgcacgcgtgagggctgcgtgtcctttggcttcttgtgggttcgcagcgcgcgtgccctgaaagcggaactggcgcaaaaacgcggtagcagcagctctggtgcataa

SEQ ID NO: 19 - protein, 175 aa, Pantoea stewartii
MLWIWNALIVFVTVVGMEVVAALAHKYIMHGWGWGWHLSHHEPRKGAFEVNDLYAVVFAIVSIALIYFGSTGIWPLQWIGAGMTAYGLLYFMVHDGLVHQRWPFRYIPRKGYLKRLYMAHRMHHAVRGKEGCVSFGFLYAPPLSKLQATLRERHAARSGAARDEQDGVDTSSSGK

SEQ ID NO: 20 - DNA, 528 bp, Pantoea stewartii
atgttgtggatttggaatgccctgatcgtgtttgtcaccgtggtcggcatggaagtggttgctgcactggcacataaatacatcatgcacggctggggttggggctggcatctttcacatcatgaaccgcgtaaaggcgcatttgaagttaacgatctctatgccgtggtattcgccattgtgtcgattgccctgatttacttcggcagtacaggaatctggccgctccagtggattggtgcaggcatgaccgcttatggtttactgtattttatggtccacgacggactggtacaccagcgctggccgttccgctacataccgcgcaaaggctacctgaaacggttatacatggcccaccgtatgcatcatgctgtaaggggaaaagagggctgcgtgtcctttggttttctgtacgcgccaccgttatctaaacttcaggcgacgctgagagaaaggcatgcggctagatcgggcgctgccagagatgagcaggacggggtggatacgtcttcatccgggaagtaa

SEQ ID NO: 21 - protein, 175 aa, Pantoea ananatis
MLWIWNALIVFVTVIGMEVVAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKLQATLRERHGARAGAARDAQGGEDEPASGK

SEQ ID NO: 22 - DNA, 528 bp, Pantoea ananatis
atgttgtggatttggaatgccctgatcgttttcgttaccgtgattggcatggaagtggttgctgcactggcacacaaatacatcatgcacggctggggttggggatggcatctttcacatcatgaaccgcgtaaaggtgcgtttgaagttaacgatctttatgccgtggtttttgctgcattatcgatcctgctgatttatctgggcagtacaggaatgtggccgctccagtggattggcgcaggtatgacggcgtatggattactctattttatggtgcacgacgggctggtgcatcaacgttggccattccgctatattccacgcaagggctacctcaaacggttgtatatggcgcaccgtatgcatcacgccgtcaggggcaaagaaggttgtgtttcttttggcttcctctatgcgccgcccctgtcaaaacttcaggcgacgctccgggaaagacatggcgctagagcgggcgctgccagagatgcgcagggcggggaggatgagcccgcatccgggaagtaa

SEQ ID NO: 23 - protein, 162 aa, Paracoccus sp. N81106
MTNFLIVVATVLVMELTAYSVHRWIMHGPLGWGWHKSHHEEHDHALEKNDLYGLVFAVIATVLFTVGWIWAPVLWWIALGMTVYGLIYFVLHDGLVHQRWPFRYIPRKGYARRLYQAHRLHHAVEGRDHCVSFGFIYAPPVDKLKQDLKMSGVLRAEAQERT

SEQ ID NO: 24 - DNA, 489 bp, Paracoccus sp. N81106
atgaccaatttcctgatcgtcgtcgccaccgtgctggtgatggagttgacggcctattccgtccaccgctggatcatgcacggccccctgggctggggctggcacaagtcccaccacgaggaacacgaccacgcgctggaaaagaacgacctgtacggcctggtctttgcggtgatcgccacggtgctgttcacggtgggctggatctgggcgccggtcctgtggtggatcgccttgggcatgactgtctatgggctgatctatttcgtcctgcatgacgggctggtgcatcagcgctggccgttccgttatatcccgcgcaagggctatgccagacgcctgtatcaggcccaccgcctgcaccatgcggtcgaggggcgcgaccattgcgtcagcttcggcttcatctatgcgcccccggtcgacaagctgaagcaggacctgaagatgtcgggcgtgctgcgggccgaggcgcaggagcgcacgtga

SEQ ID NO: 25 - protein, 309 aa, Tagetes erecta
MAAAIAVPCSSRPFGLGRMRLLGHKPTTITCHFPFSFSIKSFTPIVRGRRCTVCFVAGGDSNSNSNNNSDSNSNNPGLDLNPAVMNRNRLVEEKMERKKSERFTYLVAAIMSTFGITSMAVMAVYYRFSWQMEGGEIPYVEMFGTFALSVGAAVGMEYWARWAHEALWHASLWHMHESHHKPREGPFELNDVFAITNAVPAIALLSYGFFHKGIIPGLCFGAGLGITVFGMAYMFVHDGLVHRRFQVGPIANVPYLRRVAAAHQLHHTEKFNGVPYGLFLGPKELEEVGGTEELDKEIQRRIKLYNNTK

SEQ ID NO: 26 - DNA, 996 bp, Tagetes erecta
ggcacgagcctctctctatttttacacttcaatggcggcagcaattgctgtcccttgtagctcaagaccatttggcttaggtcgaatgcggttacttggtcataaacccacaaccataacttgtcacttccccttttctttttctatcaaatcatttaccccaattgttaggggcagaagatgtactgtttgttttgttgccggtggcgacagtaatagtaacagtaataataatagtgacagtaatagtaataatccgggtctggatttaaacccggcggttatgaaccgtaaccgtttggttgaagaaaaaatggagaggaaaaaatcggaacgatttacttatcttgttgcagctattatgtctacttttggaattacttcaatggcggttatggcggtttattaccggttttcatggcaaatggagggtggagaaattccttatgtggagatgtttggtacatttgctctctccgttggtgctgcggtaggaatggagtattgggcaagatgggctcatgaggcactatggcatgcttctttgtggcacatgcatgagtcacaccataagccacgagaaggtccgtttgagcttaatgatgtgtttgctataacaaatgcggtcccggccattgcgttgcttagttatgggtttttccacaaaggcataattccgggtctttgttttggggcgggactgggaattacggtgtttggaatggcgtatatgttcgtccacgacgggctagttcacagaagattccaagtgggtccgattgcgaatgttccctatcttcgaagggttgcagcggctcatcagctgcatcacacggaaaaatttaatggtgttccttatggcttgttcttgggacctaaggagctagaagaagtgggtggtacggaagaattggacaaggagattcaaagaagaattaaattgtataataatactaaataaataaattttgtataaaattaatataatttaatgat

SEQ ID NO: 27 - protein, 244 aa, Brevundimonas sp. SD212
MTAAVAEPRIVPRQTWIGLTLAGMIVAGWGSLHVYGVYFHRWGTSSLVIVPAIVAVQTWLSVGLFIVAHDAMHGSLAPGRPRLNAAVGRLTLGLYAGFRFDRLKTAHHAHHAAPGTADDPDFYAPAPRAFLPWFLNFFRTYFGWREMAVLTALVLIALFGLGARPANLLTFWAAPALLSALQLFTFGTWLPHRHTDQPFADAHHARSSGYGPVLSLLTCFHFGRHHEHHLTPWRPWWRLWRGES

SEQ ID NO: 28 - DNA, 735 bp, Brevundimonas sp. SD212
atgaccgccgccgtcgccgagccccgcatcgtcccgcgccagacctggatcggtctgaccctggcgggaatgatcgtggcgggatgggggagcctgcacgtctacggcgtctattttcaccgctggggcacctccagtctggtgatcgtcccggcgatcgtagcggtccagacctggttgtcggtcggccttttcatcgtcgcccatgacgccatgcacggctccctggcgccgggacggccgcggctgaacgccgcagtcggccggctgaccctggggctctatgcgggcttccgcttcgatcggctgaagacggcgcaccacgcccaccacgccgcgcccggcacggccgacgacccggacttttacgccccggcgccccgcgccttccttccctggttcctgaacttctttcgcacctatttcggctggcgcgagatggcggtcctgaccgccctggtcctgatcgccctcttcggcctgggggcgcggccggccaatctcctgaccttctgggccgcgccggccctgctttcagcgcttcagctcttcaccttcggcacctggctgccgcaccgccacaccgaccagccgttcgccgacgcccaccacgcccgcagcagcggctacggccccgttctttccctgctcacctgcttccacttcggccgccaccacgaacaccacctcaccccctggcggccctggtggcgtttgtggcgcggcgagtcttga

SEQ ID NO: 29 - protein, 242 aa, Paracoccus sp. PC1
MSGRKPGTTGDTIVNLGLTAAILLCWLVLHAFTLWLLDAAAHPLLAVLCLAGLTWLSVGLFIIAHDAMHGSVVPGRPRANAAIGQLALWLYAGFSWPKLIAKHMTHHRHAGTDNDPDFGHGGPVRWYGSFVSTYFGWREGLLLPVIVTTYALILGDRWMYVIFWPVPAVLASIQIFVFGTWLPHRPGHDDFPDRHNARSTGIGDPLSLLTCFHFGGYHHEHHLHPHVPWWRLPRTRKTGGRA

SEQ ID NO: 30 - DNA, 729 bp, Paracoccus sp. PC1
atgtccggacggaagcctggcacaactggcgacacgatcgtcaatctcggtctgaccgccgcgatcctgctgtgctggctggtcctgcacgcctttacgctatggttgctagatgcggccgcgcatccgctgcttgccgtgctgtgcctggctgggctgacctggctgtcggtcgggctgttcatcatcgcgcatgacgcaatgcacgggtccgtggtgccggggcggccgcgcgccaatgcggcgatcgggcaactggcgctgtggctctatgcggggttctcgtggcccaagctgatcgccaagcacatgacgcatcaccggcacgccggcaccgacaacgatcccgatttcggtcacggagggcccgtgcgctggtacggcagcttcgtctccacctatttcggctggcgagagggactgctgctaccggtgatcgtcaccacctatgcgctgatcctgggcgatcgctggatgtatgtcatcttctggccggtcccggccgttctggcgtcgatccagattttcgtcttcggaacttggctgccccaccgcccgggacatgacgattttcccgaccggcacaacgcgaggtcgaccggcatcggcgacccgttgtcactactgacctgcttccatttcggcggctatcaccacgaacatcacctgcatccgcatgtgccgtggtggcgcctgcctcgtacacgcaagaccggaggccgcgcatga

SEQ ID NO: 31 - protein, 242 aa, Paracoccus sp. N81106
MSAHALPKADLTATSLIVSGGIIAAWLALHVHALWFLDAAAHPILAIANFLGLTWLSVGLFIIAHDAMHGSVVPGRPRANAAMGQLVLWLYAGFSWRKMIVKHMAHHRHAGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 32 - DNA, 729 bp, Paracoccus sp. N81106
atgagcgcacatgccctgcccaaggcagatctgaccgccaccagcctgatcgtatcgggcggcatcatcgccgcttggctggccctgcatgtgcatgcgctgtggtttctggacgcagcggcgcatcccatcctggcgatcgcaaatttcctggggctgacctggctgtcggtcggattgttcatcatcgcgcatgacgcgatgcacgggtcggtggtgccggggcgtccgcgcgccaatgcggcgatgggccagcttgtcctgtggctgtatgccggattttcgtggcgcaagatgatcgtcaagcacatggcccatcaccgccatgccggaaccgacgacgaccccgatttcgaccatggcggcccggtccgctggtacgcccgcttcatcggcacctatttcggctggcgcgaggggctgcttctgcccgtcatcgtgacggtctatgcgctgatccttggggatcgctggatgtacgtggtattctggccgctgccgtcgatcctggcgtcgatccagctgttcgtgttcggcacctggctgccgcaccgccccggccacgacgcgttcccggaccgccacaatgcgcggtcgtcgcggatcagcgaccccgtgtcgctgctgacctgttttcactttggcggttatcatcacgaacaccacctgcacccgacggtgccgtggtggcgcctgcccagcacccgcaccaagggggacaccgcatga

SEQ ID NO: 33 - protein, 329 aa, Haematococcus pluvialis
MQLAATVMLEQLTGSAEALKEKEKEVAGSSDVLRTWATQYSLPSEESDAARPGLKNAYKPPPSDTKGITMALRVIGSWAAVFLHAIFQIKLPTSLDQLHWLPVSDATAQLVSGTSSLLDIVVVFFVLEFLYTGLFITTHDAMHGTIAMRNRQLNDFLGRVCISLYAWFDYNMLHRKHWEHHNHTGEVGKDPDFHRGNPGIVPWFASFMSSYMSMWQFARLAWWTVVMQLLGAPMANLLVFMAAAPILSAFRLFYFGTYMPHKPEPGAASGSSPAVMNWWKSRTSQASDLVSFLTCYHFDLHWEHHRWPFAPWWELPNCRRLSGRGLVPA

SEQ ID NO: 34 - DNA, 990 bp, Haematococcus pluvialis
atgcagctagcagcgacagtaatgttggagcagcttaccggaagcgctgaggcactcaaggagaaggagaaggaggttgcaggcagctctgacgtgttgcgtacatgggcgacccagtactcgcttccgtcagaagagtcagacgcggcccgcccgggactgaagaatgcctacaagccaccaccttccgacacaaagggcatcacaatggcgctacgtgtcatcggctcctgggccgcagtgttcctccacgccatttttcaaatcaagcttccgacctccttggaccagctgcactggctgcccgtgtcagatgccacagctcagctggttagcggcacgagcagcctgctcgacatcgtcgtagtattctttgtcctggagttcctgtacacaggcctttttatcaccacgcatgatgctatgcatggcaccatcgccatgagaaacaggcagcttaatgacttcttgggcagagtatgcatctccttgtacgcctggtttgattacaacatgctgcaccgcaagcattgggagcaccacaaccacactggcgaggtgggcaaggaccctgacttccacaggggaaaccctggcattgtgccctggtttgccagcttcatgtccagctacatgtcgatgtggcagtttgcgcgcctcgcatggtggacggtggtcatgcagctgctgggtgcgccaatggcgaacctgctggtgttcatggcggccgcgcccatcctgtccgccttccgcttgttctactttggcacgtacatgccccacaagcctgagcctggcgccgcgtcaggctcttcaccagccgtcatgaactggtggaagtcgcgcactagccaggcgtccgacctggtcagctttctgacctgctaccacttcgacctgcactgggagcaccaccgctggcccttcgccccctggtgggagctgcccaactgccgccgcctgtctggccgaggtctggttcctgcctag

SEQ ID NO: 35 - protein, 255 aa, Erythrobacter litoralis
MSDGPAFSLPARSRQQAIGLTLAVLIAGAWLGIHAYAMFVFELSWQNLPFALLLATVQTWLSVGVFIVSHDAMHGSLAPGRPTLNSAIGAFLLALYAGFGWRRMRDAHFTHHKLAGHAGDPDFDEHNPRSFWRWYGTFFRRYFSWQSILFVHVVVGIYWLVLDIPMVQIVLLYGAPALLSSLQLFYFGTFRPHRRSEEGFADRHNARSDGFGTLASLATCFHFGYHLEHHRRPDVPWWALPGAREAGIGMEAQNA

SEQ ID NO: 36 - DNA, 768 bp, Erythrobacter litoralis
atgtctgacggtcctgccttttcgttgcctgcgcgctcgcgtcagcaggcgatcgggttgacgctcgccgtgctgatcgctggcgcgtggctcggcatccacgcctatgcgatgttcgttttcgagctgagttggcagaacctgcccttcgcacttctgctagcaacggtgcaaacgtggctgtcggttggcgtcttcatcgttagtcatgacgcaatgcacggctcgctggcacccggcaggccgacgctgaattcggcgatcggcgcgtttctcttggccctttacgcaggcttcggctggcgacgaatgcgtgacgcgcattttacccaccacaagctggcgggccatgcgggagacccggatttcgacgagcacaatccgcgcagtttctggcgctggtacggaaccttcttccggcgctatttcagctggcaatcgatcctgttcgtgcatgtcgttgtcggcatctactggctagtcctcgacattccgatggtgcagatcgtcctgctctacggcgcaccggcactgctttcatcgctgcagcttttctacttcgggacctttcgtccacatcggcgttccgaagagggttttgccgaccggcacaacgcgcgcagcgatggcttcggcacgttggccagcctcgccacgtgcttccatttcggctatcatctcgaacatcatcggcgtcccgacgtgccgtggtgggccttgcccggcgcgcgagaagcggggatcggaatggaagcgcaaaacgcatga

SEQ ID NO: 37 - protein, 350 aa, Sphingomonas sp. PB304
MAGMSPSRMNLPEKSRPPVAAAISGMMMSSVRLATMVPKAAPMITPTARPSTLPRVMKDRKSLSMQFLPPEGLKKGRQAQAGRSLVCPGVTDVARTAAHGYIAAMTGDGERRRQTLVGLGLAAGIIAAFVLLHVSAVFFVPLNGGWLLAAPLLVAVLSWLSVGLFIVAHDAMHGSLAPGRPGVNLFFGRLTLLLYAGFWMDRLRPKHFDHHKHVGTEHDPDFSADHPTRFWPWYLTFMKRYFGLREYLILNVLVLAYVVVLKAPIANLLLYWALPAILSSLQLFYFGTYLPHRHEDAAFADEHKARSNDYPAWLSLLTCFHFGYHREHHLSPGTPWWALPTKRRQLQLPA

SEQ ID NO: 38 - DNA, 1053 bp, Sphingomonas sp. PB304
tcaggcgggaagctgaagttggcggcgcttggtcggaagcgcccaccagggcgttccggggctcagatgatgctcgcgatggtagccgaagtggaagcaggtgagcagcgacagccaggccggatagtcgttgcttcgggccttgtgctcgtcggcgaaggcggcgtcctcgtgccggtgcggaagataggtgccgaaatagaatagctgcagcgaggacaggatggccggcagcgcccagtagaggaggagattggcgattggagccttgagcacgaccacataggcgagcaccagtacgttcaggatcagatactcgcgaagcccgaagtagcgcttcatgaaggtcaggtaccagggccagaaacgggtcgggtgatcggccgaaaagtccggatcatgctcggtgccgacatgcttgtgatggtcgaaatgcttcggccgcagacggtccatccagaagcccgcatagagcaggagggtgagccgcccgaaaaagaggttcacgcccggccggcccggcgcaaggcttccgtgcatcgcgtcatgggcgacgatgaacagcccgacggacagccagctcagcaccgccaccagcaaaggagcggcgagcagccagcccccgttgagcggaacgaagaagacggcgcttacatggagcaggacgaaggcagcaatgatcccggcggcgaggccgagccccaccaacgtctgccgccgccgttcaccatcacctgtcatggcggcgatatagccatgagcggccgttcgtgccacgtctgtcacgccgggacagaccaaagaccgcccggcctgtgcttggcgtcccttcttcaatccttccggggggaggaactgcatgctcagggatttccgatctttcatcacgcgcggcaacgtgctcggccttgcggtcggcgtgatcatcggcgccgccttcggcaccattgtcgcaagcctgaccgaagacatcatcatgccgctgatcgctgcggcgacaggaggtctcgacttttccggcaagttcatcctgctgggcgacattccggccac

SEQ ID NO: 39 - protein, 258 aa, Bradyrhizobium sp. ORS 278
MHAATAKATEFGASRRDDARQRRVGLTLAAVIIAAWLVLHVGLMFFWPLTLHSLLPALPLVVLQTWLYVGLFIIAHDCMHGSLVPFKPQVNRRIGQLCLFLYAGFSFDALNVEHHKHHRHPGTAEDPDFDEVPPHGFWHWFASFFLHYFGWKQVAIIAAVSLVYQLVFAVPLQNILLFWALPGLLSALQLFTFGTYLPHKPATQPFADRHNARTSEFPAWLSLLTCFHFGFHHEHHLHPDAPWWRLPEIKRRALERRD

SEQ ID NO: 40 - DNA, 777 bp, Bradyrhizobium sp. ORS 278
atgcatgcagcaaccgccaaggctactgagttcggggcctctcggcgcgacgatgcgaggcagcgccgcgtcggtctcacgctggccgcggtcatcatcgccgcctggctggtgctgcatgtcggtctgatgttcttctggccgctgacccttcacagcctgctgccggctttgcctctggtggtgctgcagacctggctctatgtaggcctgttcatcatcgcgcatgactgcatgcacggctcgctggtgccgttcaagccgcaggtcaaccgccgtatcggacagctctgcctgttcctctatgccgggttctccttcgacgctctcaatgtcgagcaccacaagcatcaccgccatcccggcacggccgaggatcccgatttcgacgaggtgccgccgcacggcttctggcactggttcgccagctttttcctgcactatttcggctggaagcaggtcgcgatcatcgcagccgtctcgctggtttatcagctcgtcttcgccgttcccttgcagaacatcctgctgttctgggcgctgcccgggctgctgtcggcgctgcagctgttcaccttcggcacctatctgccgcacaagccggccacgcagcccttcgccgatcgccacaacgcgcggacgagcgaatttcccgcgtggctgtcgctgctgacctgcttccacttcggctttcatcacgagcatcatctgcatcccgatgcgccgtggtggcggctgccggagatcaagcggcgggccctggaaaggcgtgactaa

SEQ ID NO: 41 - protein, 258 aa, Nostoc sp. PCC 7120
MVQCQPSSLHSEKLVLLSSTIRDDKNINKGIFIACFILFLWAISLILLLSIDTSIIHKSLLGIAMLWQTFLYTGLFITAHDAMHGVVYPKNPRINNFIGKLTLILYGLLPYKDLLKKHWLHHGHPGTDLDPDYYNGHPQNFFLWYLHFMKSYWRWTQIFGLVMIFHGLKNLVHIPENNLIIFWMIPSILSSVQLFYFGTFLPHKKLEGGYTNPHCARSIPLPLFWSFVTCYHFGYHKEHHEYPQLPWWKLPEAHKISL

SEQ ID NO: 42 - DNA, 777 bp, Nostoc sp. PCC 7120
atggttcagtgtcaaccatcatctctgcattcagaaaaactggtgttattgtcatcgacaatcagagatgataaaaatattaataagggtatatttattgcctgctttatcttatttttatgggcaattagtttaatcttattactctcaatagatacatccataattcataagagcttattaggtatagccatgctttggcagaccttcttatatacaggtttatttattactgctcatgatgccatgcacggcgtagtttatcccaaaaatcccagaataaataattttataggtaagctcactctaatcttgtatggactactcccttataaagatttattgaaaaaacattggttacaccacggacatcctggtactgatttagaccctgattattacaatggtcatccccaaaacttctttctttggtatctacattttatgaagtcttattggcgatggacgcaaattttcggattagtgatgatttttcatggacttaaaaatctggtgcatataccagaaaataatttaattatattttggatgataccttctattttaagttcagtacaactattttattttggtacatttttgcctcataaaaagctagaaggtggttatactaacccccattgtgcgcgcagtatcccattacctcttttttggtcttttgttacttgttatcacttcggctaccacaaggaacatcacgaataccctcaacttccttggtggaaattacctgaagctcacaaaatatctttataa

SEQ ID NO: 43 - protein, 444 aa, Chlamydomonas reinhardtii
MGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALAPGPLPVPAAAAATAATAAAAAAATGSPAPASRAGSASSASAAASGFGSGHSGSVAAQPLSSLPLLSEGVKGLVEGAMELVAGGSSSGGGGEGGKPGAGEHGLLQRQRQLAPVGVMA

SEQ ID NO: 44 - DNA, 1335 bp, Chlamydomonas reinhardtii
atgggccctgggatacaacccacttccgcgcgaccgtgttctaggaccaaacacagtcgatttgcgctacttgccgcagcgctgaccgcacgacgcgtcaagcagttcacgaagcagttccgctcgcgtaggatggcggaggacatactgaagctgtggcagcgccaatatcacctgccgcgcgaggattctgacaagcgcacgctgcgcgagcgcgttcacctgtaccgcccgccgcgttcagacctaggtggcattgcggtcgctgtgacagtcatcgcgctgtgggcgacgctgtttgtctacgggctgtggttcgtcaagctgccatgggcgctcaaagtgggcgagacagccacgtcctgggcaaccattgctgctgtattctttagcctggaattcctttacaccgggctcttcatcaccacgcacgacgcgatgcatggcaccatcgcgctgcgcaaccggcgcctgaacgactttctgggccagctggcaatcagcctatacgcctggtttgactactccgtcctgcaccgcaagcactgggagcaccacaaccacaccggggagccgcgtgtggatccggacttccaccgcggcaaccccaacctggcggtgtggttcgcgcagttcatggtgtcgtacatgaccctcagccagttcctcaagatcgcggtctggtccaacctgctgctgctggcgggtgcgccgctggccaaccagctgctgttcatgacggcggcgcccatcctgtccgccttccgcctgttctactacggcacctacgtgccgcaccacccggagaaggggcacaccggcgccatgccctggcaggtatcccgcaccagctccgcctcccggctgcagtcgttcctcacctgctaccacttcgacctgcactgggagcaccaccgctggccctacgcgccctggtgggagctgcccaagtgccgccagattgcccgcggcgcagccctggcgcccgggccgctgcccgtgccggcagcggcggcggctacagccgccaccgcggcggcggcagcagcagctacaggcagccccgctcccgccagccgagcagggtcagcttcctccgcctccgcagcggcctccggatttggatccggacacagcggctctgtggctgcgcagccgctgtcttcgctgcccttgctgagcgagggcgtgaaggggttggtggagggggcgatggagttggtggcaggtggcagcagcagcggcggtggtggagagggcggcaagccgggcgcgggggagcacgggctgctgcagcggcagcggcagctggcgcctgttggcgtgatggcttga

SEQ ID NO: 45 - protein, 328 aa, Chlamydomonas reinhardtii
MGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALA

SEQ ID NO: 46 - DNA, 987 bp, Chlamydomonas reinhardtii
atgggccctgggatacaacccacttccgcgcgaccgtgttctaggaccaaacacagtcgatttgcgctacttgccgcagcgctgaccgcacgacgcgtcaagcagttcacgaagcagttccgctcgcgtaggatggcggaggacatactgaagctgtggcagcgccaatatcacctgccgcgcgaggattctgacaagcgcacgctgcgcgagcgcgttcacctgtaccgcccgccgcgttcagacctaggtggcattgcggtcgctgtgacagtcatcgcgctgtgggcgacgctgtttgtctacgggctgtggttcgtcaagctgccatgggcgctcaaagtgggcgagacagccacgtcctgggcaaccattgctgctgtattctttagcctggaattcctttacaccgggctcttcatcaccacgcacgacgcgatgcatggcaccatcgcgctgcgcaaccggcgcctgaacgactttctgggccagctggcaatcagcctatacgcctggtttgactactccgtcctgcaccgcaagcactgggagcaccacaaccacaccggggagccgcgtgtggatccggacttccaccgcggcaaccccaacctggcggtgtggttcgcgcagttcatggtgtcgtacatgaccctcagccagttcctcaagatcgcggtctggtccaacctgctgctgctggcgggtgcgccgctggccaaccagctgctgttcatgacggcggcgcccatcctgtccgccttccgcctgttctactacggcacctacgtgccgcaccacccggagaaggggcacaccggcgccatgccctggcaggtatcccgcaccagctccgcctcccggctgcagtcgttcctcacctgctaccacttcgacctgcactgggagcaccaccgctggccctacgcgccctggtgggagctgcccaagtgccgccagattgcccgcggcgcagccctggcgtga

SEQ ID NO: 47 - protein, 514 aa, Deinococcus geothermalis
MRAMPDFDVVVMGAGHNALVTAAYAAKAGLKVGVFERRHLVGGAVSTEELVPGYRFDYGGSAHILIRMTPVVRELELSRHGLHYLEVDPMFHASDGETPWFVWRDAERTARELEALFPGQGEAYRRFLEDWTPFARSVADLFNSAPGPLELGRMALRGGQGRAWQAQLPRILRPYGDVAREYFSEERVRAPLVWMAAQSGPPPTDPLSAPFLLWHPLYHVGGVARPKGGSGGLTQALRRAVEAGGGQVLTNAPVKRILVKGGRAQGIELAGGERYTARAVVSGTHILATAGALPPEHVPEAARRVRVGDGFGMVLRLALSERVRYRQHREPESRVGLGLLIKHERQLLRGYGEYLAGEPTRDPPLVAMSFSAVDDSLAPPGGEVLWLWAQYYPFELASGTWESRQAEARENILRAFEHYAPGTRDSIVGELVQTPQWLERELGLLRGNVMHLEMSFDQMFAFRPWLGASQYRWPGVRGLYLTGASTHPGGGIMGASGRNAARVLLRDLTRRRGT

SEQ ID NO: 48 - DNA, 1545 bp, Deinococcus geothermalis
atgcgggctatgccggacttcgacgttgtggtgatgggcgcggggcacaacgcgctggtgacggccgcctacgcggccaaggccggactgaaggtcggcgtatttgagcggcggcatctggtgggcggcgcggtgagcaccgaggaactcgtccccggttaccgctttgactacggcggcagcgcccacatcctgattcgtatgacgccggtggtgcgcgagctggaactctcgcgccacggcctgcattacctggaagtggacccgatgttccacgcctcggacggcgagacgccttggtttgtctggcgcgacgccgagcgcacggcacgggagctggaggccctgtttcccggccagggcgaggcgtaccgccgctttctggaagactggacgccctttgcgcgcagcgtcgcggacctcttcaactcggcccccggtccgctggagctggggaggatggccctgcggggcgggcaggggagagcctggcaggcccagcttccccgcatcctgcgcccttacggcgacgtggcccgcgagtatttttcagaggagcgggtgcgggcaccgctggtctggatggcggcccagagtggcccgccccccacggatccgctcagtgcccccttcttgctgtggcaccccctctaccacgtgggcggcgtggcgcggcccaagggaggaagcggcggactgacccaggccctgcgccgggccgtggaggcgggcggcggccaggttttgaccaacgcgccggtaaaacgcatcctggtgaaaggtggccgggcgcagggcatcgagctggcgggcggcgagcggtacaccgctcgagcggtcgtgtccggaacccacatcctcgcgacggcaggggcactgccgcctgaacacgtgcccgaggcagcgcgcagggtccgggtgggggacggctttgggatggtgctgcggctcgcccttagcgaacgggtgcgctaccgccaacaccgtgagccggaaagccgggtggggctgggcctgctgatcaagcatgagcggcagctcctgcggggctacggcgaatacttggcgggcgaacccacccgcgatccgcccctggtcgccatgagcttttcggcggtggacgacagcctggcgccgcccggcggcgaggtgctgtggctgtgggcgcagtattacccctttgagctggcgtcgggaacgtgggagagccgccaagcagaagcgcgtgagaacattctgcgggcctttgaacactacgcccctgggacacgggacagcattgtgggcgaactggtgcagacgccgcagtggctggaaagggagctgggcctcttgcgcggcaacgtgatgcacctggagatgagttttgaccagatgttcgcttttcgcccctggttgggggccagccagtaccggtggccgggcgtgcggggcctttacctcaccggcgcgagcacccaccctggcggcggcatcatgggggcgtcaggcaggaacgcggcgcgggttctgttgcgggatctaacacgccggagggggacatga

SEQ ID NO: 49 - protein, 329 aa, Deinococcus geothermalis
MHPDLLPRVLSLLPNRGDRPELRAFSAMLRDYPERGGKGIRSELLLASARAHGALPSTSAWEGALWLAAGLELFQNWVLIHDDIEDDSEERRGRPALHRLYGVPVAINVGDALHASMWAAVHRAGVPGGLEEFLNMVWRTAEGQHLDLTWVQERSWNLGEADYLAMVRLKTALYTVVVPLRLGALAAGVLPDERFTAAGEALGAAFQIRDDVLNLAGDPAKYGKEIGGDLWEGKRTLIVLHWLAHAPEEQRQVFLEQMRRDRADKDAAAIAAIHRWLLESGSVQYAQAYADTQAREGLARLTEALENAPDPQAARALLAQLRGLATREA

SEQ ID NO: 50 - DNA, 990 bp, Deinococcus geothermalis
atgcaccccgacctgctcccccgcgtgctgtctctgcttcccaaccgcggtgaccgcccggaactgcgcgccttctccgccatgctgcgcgactatcccgagcgcggcggaaagggcatccgctcggaactgctgctcgccagtgcccgcgcccatggcgccttgcccagcacttcagcctgggaaggagcgctgtggctcgcggcgggcctggaactgttccagaattgggtgctgatccacgacgacatcgaggatgactcggaggagcgccggggcaggcccgcgctgcaccgcctgtacggcgtgccggtagccatcaatgtgggtgacgccctccacgcctcgatgtgggccgccgtccaccgcgcgggggtgccgggcggcctggaagaattcctgaacatggtctggcgcacggcggagggccagcacctcgatctcacctgggtgcaggagcgcagctggaacctgggcgaagccgactatctggcgatggtgcgcctgaaaacggccctgtacacggtggtggtgccgctgcggctcggggcactcgccgccggagtgctcccggacgagcgctttacggcagcgggcgaagccctgggcgcggccttccaaattcgcgacgacgtgctgaacctggccggggatcccgcgaagtacggcaaggagatcggcggcgacctctgggagggcaaacgcaccttgattgtgctgcactggctcgcccacgcgccggaggagcagcggcaggtctttctggaacagatgcgccgggaccgagcggacaaggacgccgccgccatcgcagccatccaccgctggctgctggagagcggcagcgttcagtacgcccaggcctacgccgacacacaggcccgcgaaggcctcgcccgcctgaccgaggcgctggagaacgcacccgacccgcaggcggcgcgggcactcctggcccagttgcgggggctggcgacgcgcgaagcgtag

SEQ ID NO: 51 - protein, 329 aa, Deinococcus radiodurans
MRPELLARVLSLLPETSATPELARFYALLRDYPQRGGKGIRSELLLASARAHGLSESDTGWESALWLAAALELFQNWVLVHDDIEDDSEERRGRPALHHLCGMPVALNVGDALHAYMWAAVGKANVPGAFEEFLQMVYRTAEGQHLDLAWVEGREWGLRPADYLQMVGLKTAHYTVIVPLRLGALAAGMAPQDAFTPAGLALGTAFQIRDDVLNLAGDPVKYGKEIGGDLLEGKRTLIVLDWLTTAPDDRKAIFLDQMRRHRADKDPAVIDEIHRWLLESGSVEAAQDYAQAQAAEGLDLLEKALADAPDAQAAAALLASVRELATREK

SEQ ID NO: 52 - DNA, 990 bp, Deinococcus radiodurans
atgcgtcccgaactgctcgcccgcgtgctctcgctgctgcccgaaaccagcgccacgcccgaactcgcccgcttctacgcgctgctgcgcgactacccgcagcgcggcggcaagggcatccgctcggagctgctgctcgccagcgcccgcgcccacggcctgagcgagtcggacaccgggtgggagagcgccctgtggctcgccgccgcgctggaactgtttcagaactgggtgctggtccacgacgacatcgaggacgactccgaggagcgccggggccggcccgcgctgcaccacctgtgcgggatgccggtggcgctcaacgtgggcgacgcgctgcacgcctatatgtgggcggcggtgggcaaagcgaacgtgcccggcgctttcgaggaattcttgcagatggtttaccgcaccgccgaggggcagcacctcgacctcgcctgggtggagggccgcgagtgggggctgcggcccgccgactacctgcaaatggtgggcctgaaaaccgcgcactacacggtgatcgtgccgctgcggctgggggcattggcggcaggcatggcgccgcaggacgcttttaccccggcgggactggcactcggcaccgccttccagattcgggacgacgtgctcaacctcgcgggcgacccggtgaaatatggcaaggaaatcggcggcgacttgctcgaaggcaagcgcaccctgatcgtgctggactggctgaccaccgcacccgacgaccggaaggcgatttttctcgaccagatgcgccggcaccgcgccgacaaggaccccgccgtcattgacgagattcaccgctggctgctggaaagcggcagcgtggaagccgcgcaggactacgcgcaggcgcaggctgccgaggggctggacctgctggaaaaagcgttggcagacgccccggacgcgcaggccgccgccgcactgctcgccagcgtacgcgaactggcgacgcgggagaagtga

SEQ ID NO: 53 - protein, 329 aa, Deinococcus deserti
MRPDLLSRVLSLLPDGGTRPELRQFSEMLRDYPQRGGKGIRSELLLASAQAHGVAHDTDAWDQAQWLAAGLELFQNWVLIHDDIQDDSEERRGQPALHRLYGVPLAINVGDALHAYMWAAVHRAGVPGAMEEFLTMIHRTAEGQHLDLSWVEHREWKLREADYLEMVQLKTAHYTVIVPLRLGALAAGVQPASAFTNAGLALGSAFQIRDDVLNLLGDPLKYGKEIGGDLLEGKRTLIVLRWLAQAPAEQREVFLDQMRRDRAAKDPAVIAQIHRWLLDSGCVTAAQDYAAARAEEGLSLLSSALSGAPGQAAVQQILGAMRALATRDA

SEQ ID NO: 54 - DNA, 990 bp, Deinococcus deserti
atgcgtcccgatctgctttcgcgtgtgctgtcgctgcttccggacggtggcacacggcccgaactgcgccagttttccgagatgctgcgcgactacccccagcgcggcggcaaaggtattcgcagcgaactgctgctggcctcagcccaggcacacggcgttgcgcacgataccgacgcctgggatcaggcgcagtggctggctgcgggcctggagctgtttcagaactgggttctgattcacgacgacatccaggacgacagcgaggaacgccgcggtcagccggcgctgcaccgtctgtacggcgtgccgctggcgatcaacgtgggcgacgcgctgcacgcctacatgtgggctgcggtccaccgcgcaggagtgccaggtgcgatggaagagttcctgaccatgatccaccggactgctgaaggccagcacctggatctgagctgggtcgaacaccgcgaatggaagctgcgggaggccgactacctggaaatggtgcagctcaagaccgcccattacaccgtgattgtgccgctgcgcctgggagccctggctgccggcgtgcagcccgccagtgcctttacgaacgctggtctggcgctgggaagcgcctttcagattcgtgatgatgtcctgaatctgctgggcgatccgctcaagtacggcaaggaaatcggcggcgacctgctggaaggcaagaggacgctgatcgtgctgcgctggcttgcccaggctccagcagagcagcgcgaggtgtttctggaccagatgcgccgggaccgcgccgccaaggacccggccgtgatcgcgcagattcatcggtggctgctggacagcggctgcgtgaccgctgcccaggactacgcagctgcccgggccgaggagggcctgtccctgctgagctcggctctgtcaggggcgccgggtcaggctgctgtgcagcagatcctcggcgccatgcgtgcactggccacgcgggatgcctga

SEQ ID NO: 55 - protein, 298 aa, Deinococcus geothermalis
MPYPAFPSPTDSRPPAAAARHCWEVTRQHSRTFSLGSRFFPAGQREAVWAVYAACRAGDDSVDECHGNEAKQALAAWWARVQAAFAGRPGPHPIDTALAWAVSHWPIPLAAFEELHEGLRMDLEGYHYRDLEDLTLYCRRVAGVVGFMIAPICGYSGGEDTLRRALRLGQAMQLTNILRDVGEDLERGRVYLPEDLLREYGVTRAALEEGRVTPGYRALMRDLSALARAWYAEGRAGIPCLRGRARIGVQAAARAYEGILDDLARGDFDNFRRRAHVSGPRKLLMLPQAWWEVRGAPP

SEQ ID NO: 56 - DNA, 897 bp, Deinococcus geothermalis
atgccataccccgccttcccctcgcccacggattcgcgtcctccagcggcggcggcccgtcactgctgggaggtgacgcggcaacacagccggacgttctcgctgggatcgcgtttcttcccggcgggccagcgcgaggccgtgtgggcggtatacgcggcctgtcgagctggcgatgacagcgtggatgagtgccacgggaacgaggccaaacaagcccttgctgcgtggtgggcccgagttcaggctgccttcgcgggcagacccggcccgcaccccatcgacacggcgctggcctgggctgtgtcgcactggccgattcccctcgcggcttttgaagaactgcacgaaggactgaggatggacctggagggttaccactaccgcgacctggaggacctgaccctatactgccgccgcgtggcgggcgtggtgggctttatgatcgctcctatctgcggttacagcgggggtgaggatacgctgcgccgggcgctccgactgggtcaggcgatgcagctcacgaatatcctgcgcgatgtgggcgaggatctggaacgcgggcgggtgtatctgcccgaggacctgctgcgcgagtacggtgtcactcgcgctgccctggaagaaggccgggtgactcccggctaccgcgcgttgatgcgtgatctctctgcgctcgcccgcgcgtggtatgccgagggccgcgccgggattccctgcctgcggggccgcgcccggatcggcgtgcaggccgctgcccgcgcttatgagggcatcctcgacgatcttgcccggggagacttcgacaacttccgccgccgcgcccatgtgagcggcccccgcaagctgctgatgctgccccaggcatggtgggaggtgcggggagcgccaccttga

SEQ ID NO: 57 - protein, 325 aa, Deinococcus radiodurans
MRSRAGLSLRLPTRTLTVTDYSPALPCTELRRPPLAQAVRYCRDLTRQHSKTFYLGSQLFSPPERAAVWAVYAACRAGDDIVDEAGNGDRERELREWRSRIDAAFAGQPADDPISTALAWAAGRYAIPHSAFAELHEGLNMDLRGHEYRDMDDLLLYCRRVAGVVGFMVAPISGYRGGAATLNDALQLGQAMQLTNILRDVGEDLTRGRVYLPQSLLDEYGLSRAALERWGQGEPLSPAYRALMTHLGGLAREWYAAGRAGIPQLDGRGPLAVLTAARAYEGILDDLERAGYDNFGRRAYVSGRRKLLMLPQAWWELRSLGAVHG

SEQ ID NO: 58 - DNA, 978 bp, Deinococcus radiodurans
tcagccgtggaccgcgcccagcgagcgcagttcccaccacgcctgcggcagcatcagcagtttgcgccggccactcacgtaggcgcggcgcccgaaattgtcgtagccggcgcgttcgaggtcgtccagaatgccctcgtaggcgcgggcggcggtcagcaccgccagcgggccgcgtccgtcgagctgcggaatcccggcgcggccagcggcgtaccactcgcgcgccagcccgccgaggtgcgtcatcagggcgcggtaggcggggctgagcggttcgccctggccccagcgctccagcgcggcgcgtgacaggccgtattcgtcgagcaggctctgcggcaggtagacccgtccgcgcgtcaggtcctcgcccacgtcgcgcaggatgttggtgagctgcatcgcctgaccgagttgcaaggcgtcgttcagcgtcgccgcgccgccccggtagccgctgataggcgcgaccatgaaccccaccaccccggccacccggcggcagtagagcaacaggtcgtccatgtcgcggtactcgtgcccacgcaggtccatgttcaggccctcgtgcagctcggcaaaggcgctgtggggaatggcgtagcgccccgccgcccacgccagcgccgtgctgatggggtcgtccgccggttgccccgcaaacgccgcgtcaatccggctgcgccactcacggagttcgcgctcgcggtccccgtttcccgcctcgtccacgatgtcgtccccggcgcggcaggcggcgtagaccgcccacacggcggcccgctcgggtggcgaaaacagttgcgaccccagataaaacgtcttgctgtgctgccgcgtcaggtcccggcagtacctcactgcctgcgccagcggcgggcggcgcagttcggtgcagggcagggcaggtgaatagtccgtcacggtgagggtcctcgtcggcagtctaagagacaaaccggccctagacctcac

SEQ ID NO: 59 - protein, 313 aa, Deinococcus deserti
MPDLNLPGSPPLAALRWCRVMTRVHSQTFYLGSLLYPRRQRLAVWAVYAACRVGDDIADEYTGAGARVELDRWWARVCSAFCGNPGDDPMQTALAWATREYPIPLDAFAELYEGFCMDLTGQSYDSLDDLTLYCRRVAGVVGFMVAPIGGYRGGAHTLEQALMLGQAMQLTNILRDVGEDLSRGRVYLPAELLSRFGVTHADLQAGRVTPEYRALMAQLCGLAREWYARGREGIPALEGRARVGVAAAARTYEGILDDLEAHGYDNFSRRAHVPPRHKLRLLWQEYRAHSSPASACPVAWAEQQYLRLKGLRG

SEQ ID NO: 60 - DNA, 942 bp, Deinococcus deserti
atgcctgacctcaatctgcccggatctcctccactggccgccctgcgctggtgccgagtgatgacccgggtccacagccagacgttctacctgggctcgctgctgtatccacggcggcaacggctggcggtctgggcggtctatgccgcctgccgggtcggcgacgacatcgcggatgaatacaccggcgccggggccagggtagagcttgaccgctggtgggccagggtctgctccgccttctgcggtaaccccggggatgacccgatgcagaccgcactggcctgggcgacccgggaatacccgattccgctggacgcgtttgccgagttatatgagggcttctgcatggacctgacgggccagtcctacgacagcctggacgacctgaccctgtactgccggcgggtcgcgggagtcgtgggctttatggtcgcgcctatcggcggctaccgtggcggggcacacaccctggaacaggccctgatgctggggcaggccatgcagctgaccaatattctgcgggatgtgggagaggacctctcacgcgggcgggtctacctgccggctgagctgctgagccgcttcggtgtcacacacgccgacctgcaggccggcagggtcacgccggagtaccgcgcactcatggcacagctgtgtggtctggcgcgggaatggtacgccaggggtcgggagggcattccggcactggaggggcgtgcccgcgtgggggtcgcggccgccgcacgcacctacgagggaattctggatgacctggaagcccacggctacgataatttcagccggcgtgcccatgtgccgccccggcacaaactgaggctgctgtggcaggaataccgcgcccattcttcgccggcgtcggcttgcccggtggcctgggcggagcagcagtacctgcgcctcaagggattgcgcggctga

SEQ ID NO: 61 - protein, 556 aa, Deinococcus geothermalis
MTDFSTLHRRKQALIIGSGIGGLSLGIRLQSLGFETTILEKLDGPGGRAYQKRTPDGYVFDMGPTVITVPHFIEELFALERDGGWPGQPDYPAEVLNGERVRVGESGGPRTREYVRLVPILPFYRIYFDDGTFFDYDGDPASTRRQIAELAPGDLAGYERFHADAEAIFRRGFLELGYTHFGDVGTMLRVVPDLLKLDAVRTLFSFTSRYFQSPKLRQVFSFETLLIGGNPLSVPAIYAMIHFVEKTWGIHYALGGTGALVRALVRKFEELGGRIEYGAEVEQILVTDEWGKPVRRPLGPRVARGVRLKGGEQRPADLVISNGDWANTYLKRVPRQARLVNNDLRVKAARQSMSLLVIYFGFREDGRSLDLRHHNIILGPRYEALLQEIFGQKVLGADFSQYLHVPTLTDPSLAPPGHHAAYTLIPVPHNGSGLDWTVEGPRLVDRVLAFLDERGYIPGLRERLTYLDFITPDYFEGTLDSYLGNAFGPEPILAQSAYFRPHNRSEDVQNLYLVGAGVQPGAGTPSVMMSAKMTARLIAEDFGLQGAVRPVDSVRR

SEQ ID NO: 62 - DNA, 1671 bp, Deinococcus geothermalis
atgactgatttctccactcttcacagacgcaaacaggccctcatcatcggctcagggataggtggcctgagtcttggcattcgccttcagagccttggcttcgagaccaccatcctggagaaactggacgggccgggcgggcgggcctaccaaaaacggacgccagatggctatgtcttcgacatggggccgaccgtcatcacggtgccgcatttcatcgaggaactgtttgcgctggaacgggacggggggtggcccgggcagcccgattacccggctgaggtgctgaacggcgaacgggtccgggtgggggaaagcggcggtccccgcacccgcgagtatgtgcggctggtgccgattctgcccttctaccgcatctatttcgacgacggcacctttttcgactatgacggcgaccccgccagtactcggcgccagattgccgaacttgcgcccggcgacctggcgggctacgaacgctttcacgcggacgcggaggcgatcttccggcgtggctttctggagctgggctacacgcatttcggtgacgtgggcaccatgctgcgcgttgtgcccgacctcctgaagctggacgccgtgcggacgctcttctcctttaccagccggtacttccagtcgcccaagctgcggcaggtcttctccttcgagacgctgctgatcggcggcaatccgctgagcgtccccgccatctacgcgatgattcactttgttgagaaaacctggggcattcactacgcgctgggcgggaccggcgccctggtgcgggccctggtgcgcaagttcgaggaactcggcgggcgcatcgagtacggcgcggaggtggagcagattctggtgacggacgagtggggcaagcctgtccggcgtccgctcggcccacgggtggcgcgtggcgtgcggctgaaggggggtgagcagcgtcccgccgacctggtgatcagcaacggggactgggccaacacctatctcaaacgggtgccccggcaggcccgcctggtcaacaatgacctgcgcgtcaaggcggctcgccagagcatgagcctgctggtgatctatttcggctttcgcgaggacggcaggtcgctcgacctgcgccaccacaacatcatcctggggccgcgctacgaggctctgctgcaagagatcttcggccagaaggtgctgggggccgactttagccagtacctccacgtgcccaccctcacggatcccagcttggcgccgcccggtcaccatgccgcctataccctgattcctgtgccgcacaacggcagtggtctcgactggacagtggaggggccgcggctggtggatcgcgtgctggcttttctggacgaacgcgggtacattccggggctgcgcgagcggctcacgtacctagacttcatcacccccgattacttcgagggcacgctcgatagctacctaggaaacgcttttggcccggaaccgattctggcccagagtgcctactttcgtccgcacaaccgctcagaggacgtgcagaacctctaccttgttggcgcgggcgtgcagcccggcgcaggcacacccagcgtgatgatgagcgccaagatgaccgcccggctgatcgcggaggacttcgggcttcagggggcagtgcggcccgtggacagcgtccggcgctag

SEQ ID NO: 63 - protein, 548 aa, Deinococcus radiodurans
MTSALPRPAPSPYARRKTALVIGAGFGGLALGIRLQSLGFDTTILERLDGPGGRAYQKRTPDGYVFDMGPTVLTVPHFIEELFALERDRAGLDAPDYPPEVLSGERVKEGVSGGPHTSRYVTLVPILPFYRIVFHDGTYFDYDGDPESTRRQIAELAPGDLAGYERFHADAEAIFRRGFLELGYTHFGDVPTMLRVVPDLLKLDAVRTLFSFTSKYFQSDKLRQVFSFETLLVGGNPLSVPAIYAMIHFVEKTWGIHYAMGGTGALVRGLVQKFEELGGAIRYGAGVDEVLVDGNLPGKRTARGVRLESGEELRADLVASNGDWANTYLKRVRPSARLVNSDLRVKAASESMSLLVVYFGFRGGDDLPLKHHNILLGPRYEALLSEIFGTKRLGEDFSQYLHVPTLTDPALAPAGHHAAYTLVPVPHNGSGIDWDVEGPKLAEAALADIERRGLIPGLRERLTHFEFITPDYFAGTLDSYLGNAFGPEPRLVQSAFFRPHNRSEDLHNFYLVGAGAQPGAGTPSVMMSAKMTARLIAEDFGIHADIRR

SEQ ID NO: 64 - DNA, 1647 bp, Deinococcus radiodurans
tcagcgccggatgtccgcgtgaatcccgaagtcctcggcgatcaggcgggccgtcatcttggccgacatcatcacgctcggcgtcccggcgcccggctgcgcgcccgcgccgacgaggtagaagttgtgcaggtcctcgctgcggttgtgcgggcggaaaaaagcgctctggacaagccgtggctccggcccgaaggcgttgccgaggtagctgtcgagcgtgcccgcgaagtagtcgggcgtgatgaactcgaagtgggtcaggcgctcacgcagccccggaatcaggccgcgccgttcgatgtcggccagggcagcttcggccagctttggcccttccacgtcccagtcgatgccgctgccgttgtggggcaccggcaccagcgtgtaggcggcgtggtgcccggcgggggccagcgcggggtcggtcagcgtgggcacatgcaggtactggctgaaatcctcgccgagccgcttggtcccgaaaatctcgctcagcagcgcctcgtagcgcggccccagcaggatgttgtggtgcttgagcggcagatcgtcgccgccgcgaaagccgaaatagacgacgagcaggctcatgctttcgctggcggccttcacccgcaggtccgaattgaccagccgcgccgagggacgcacgcgcttgaggtaggtgttcgcccagtcgccgttgctcgccaccaggtcggcccgcagctcttcgccgctctccagccgcacaccgcgcgctgtgcgtttgccgggtagattgccgtccaccagcacctcgtctacgccggcgccgtagcggatggctccgccgagctcctcaaatttctgcaccagcccgcgcaccagcgccccggtgccgcccatcgcgtagtggatgccccaggtcttctcgacgaagtgaatcatcgcgtagatggcgggcacgctcagcggattgccgccgacgagcagcgtctcgaaggaaaagacctgccgcagtttgtccgactggaagtatttagacgtgaacgaaaagagggtgcgcaccgcgtcgagcttcagcaggtcgggcaccacccgcagcatcgtcggcacgtcgccgaaatgggtgtagccgagttccagaaagccccgccggaaaatcgcctcggcgtcggcgtgaaagcgctcatagcccgcgaggtcgcccggcgcgagttcggcaatctggcgccgggtgctctccgggtcgccgtcgtagtcgaaatacgtgccgtcatgaaagacgatgcggtagaagggcaaaatcggcaccagcgtcacgtaccggctggtgtgggggccgccgctcacgccctccttgacccgctcgccgctcagcacctcgggtgggtagtcgggcgcgtccaggccagcgcggtcccgctccagcgcgaacagttcctcgatgaaatgcggcaccgtcagcacggtcggccccatgtcgaacacatacccgtccggcgtgcgcttctggtaggcgcgaccccccggtccatcgagccgttccagaatggtggtgtcgaagcctaggctttgcagccgaatgccgagcgcgaggccgccgaagcccgcgccgatgacgagggccgtcttgcggcgggcatagggcgaaggagcagggcgaggaagtgcagatgtcat

SEQ ID NO: 65 - protein, 550 aa, Deinococcus deserti
MARPPELTRRKVAVIIGAGIGGLSLGIRLQSLGFDTTILERLDGPGGRGYQKRTPDGYVFDMGPTVITVPHFIEELFALERDRAALGAEDFPPAVRQAPRVTSGESGGPRTRDYVKLVPILPFYRIVFDDGSYFDYDGDPEGTRRQIAALAPEDLEGYERFHRDAQAIFERGFLELGYTHFGDVPTMLGAVPDLLRLDAVRTLFSFTSKYFRSDKMRQVFSFETLLVGGNPLSVPAIYAMIHFVEKTWGIHYAMGGTGALVQAMVRKFEELGGTVRYDAGVEEILVTDEWGQLVRTPLGRRQARGVRLESGETVPADLVVSNGDWANTYLRRVPRAARLVNSDLRVRAARQSMSLLVIYFGFRDNGRPLGLRHHNILLGPRYEALLREIFGQKVLGADFSQYLHVPTLTDPSLAPEGHHAAYTLVPVPHNGSGLDWTVQGPLLVDRVLNYLEERGVIPDLRARLTHLEYVTPDYFETTLDSYLGNAFGPEPVLTQSAFFRPHNRSEDVSNLYLVGAGAQPGAGTPSVMMSAKMTARLIAQDFGVPAEITG

SEQ ID NO: 66 - DNA, 1653 bp, Deinococcus deserti
atggcgcgtccaccagaactcacccggcgcaaggttgccgtgattatcggggcaggcattggcgggctgagcctgggcatccgcctgcagagcctgggcttcgacacgaccattctggagcgccttgacgggcccggtggacgcgggtatcagaaacgcacgcccgacggctacgtgttcgatatgggcccgaccgtcatcacggtgccgcactttatcgaggaactgtttgcactggaacgtgaccgcgccgccctgggcgccgaagattttccgccggcggtccgtcaggcgccgagggtgacgtccggcgaaagcggtggcccccggacccgcgactacgtcaagctggtgccgatcctgccgttctaccggatcgtgtttgatgacggctcgtatttcgattacgacggtgacccagaaggaacccgccggcagatcgcggcgctggcgcccgaggatctggagggctacgagcgctttcaccgtgacgcccaggccatcttcgagcgaggattcctggagctgggctacacgcatttcggtgatgtgcccacgatgctgggcgccgtgcctgacctgctccgcctggacgccgtgcgcacactgttctcgttcacgtcaaagtacttccgctcggacaagatgaggcaggtgttttcgttcgagacgttgctggtcggcggcaacccgctgagcgttccggcgatttacgccatgattcattttgtcgagaagacctggggaatccattacgcgatgggcgggaccggggcgctggttcaggccatggtgcgcaaatttgaggagctcggcgggaccgtccgatatgacgccggggtcgaggagattctggttaccgacgagtggggtcagctggtccggactccgctgggacgtcgtcaggcgcgtggcgtgcgcctggaaagcggcgagacagtgccggccgatctggtggtcagcaacggcgactgggccaacacctacctccggcgggttccgcgcgcggcgcggctggtcaactccgacctgcgggtgcgcgccgcccgccagagcatgagcctgctggtgatctatttcggatttcgggacaacggccggccgctcgggctgcgccaccacaacattctgctgggcccgcgctatgaagccctgttgcgagagatcttcggacagaaggtgctgggggcagacttcagccagtacctgcacgtgccgaccctgaccgatccgtcccttgctcccgaagggcaccacgcggcgtacaccctggttcctgtgccgcacaacggcagcggactggactggaccgttcagggacctctgctggtggaccgcgttctgaattatctggaagaacggggcgttattccagacctgcgggcgcgcctgacgcatctggagtacgtgacgcccgattacttcgaaacgacgctggacagttatctggggaatgccttcgggcctgaaccggtgctgacccagagcgccttcttccgtccgcacaaccgcagcgaggacgtgtccaacctgtatctggtgggcgctggggcccagccgggcgccgggacgcccagtgtgatgatgtcggccaagatgaccgcgcgcctgatcgcccaggactttggagtgcctgccgaaatcacggggtag

SEQ ID NO: 67 - protein, 242 aa, Paracoccus haeundaensis
MSAHALPKADLTATSLIVSGGIIAAWLALHVHALWFLDAAAHPILAIANFLGLTWLSVGLFFIAHDAMHGSVVPGRPRGNAAMGQLVLWLYAGFSWRKMIVKHMAHHRHTGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 68 - DNA, 729 bp, Paracoccus haeundaensis
atgagcgcacatgccctgcccaaggcagatctgaccgccaccagcctgatcgtctcgggcggcatcatcgccgcgtggctggccctgcatgtgcatgcgctgtggtttctggacgcggcggcgcatcccatcctggcgatcgcgaatttcctggggctgacctggctgtcggtcggtctgttcttcatcgcgcatgacgcgatgcacgggtcggtcgtgccggggcgtccgcgcggcaatgcggcgatgggccagctggtcctgtggctgtatgccggattttcgtggcgcaagatgatcgtcaagcacatggcccatcaccgccataccggaaccgacgacgaccccgatttcgaccatggcggcccggtccgctggtacgcgcgcttcatcggcacctatttcggctggcgcgaggggctgctgctgcccgtcatcgtgacggtctatgcgctgatcctgggggatcgctggatgtacgtggtcttctggccgctgccgtcgatcctggcgtcgatccagctgttcgtgttcggcacctggctgccgcaccgccccggccacgacgcgttcccggaccgccataatgcgcggtcgtcgcggatcagcgaccccgtgtcgctgctgacctgctttcactttggtggttatcatcacgaacaccacctgcacccgacggtgccttggtggcgcctgcccagcacccgcaccaagggggacaccgcatga

SEQ ID NO: 69 - protein, 245 aa, Paracoccus sanguinis
MNPAARALRPPAATPVSVALAVAIVAAWLGLHVHAVWFLDAPAHPVRAALCLIGLTWLSVGLFIVAHDAMHGAILPGRPRLNAAIGRLALTLYAGFSWPKLMRHHMAHHRAPGSADDPDFGHGHRGIVAWYADFVRTYFGWREVFVLGGAVILYALILGPRWPYVAFWAVPSLMASVQLFVFGTFLPHRLEAGPFADRHNAASTRLAVPLSLLTCYHFGGYHHEHHLHPGVPWWALPRTRTGPRR

SEQ ID NO: 70 - DNA, 738 bp, Paracoccus sanguinis
atgaacccggccgctcgtgcactccgcccgcccgccgccacgcccgtgagcgtggcgctcgccgtggcgatcgtggcggcgtggctggggctgcacgtccatgcggtgtggtttctcgacgcgccggcccatccggtccgtgcggcgctgtgcctgatcgggctgacctggctgtcggtggggctgttcatcgtcgcccatgacgccatgcatggcgcgatcctgcccggccgcccgcgcctcaacgccgccatcgggcggctggccctgacgctctatgccgggttttcctggcccaagctgatgcgccaccacatggcccaccaccgcgcgccggggtcggccgacgacccggatttcggccacggccaccgcggcatcgtcgcgtggtacgccgatttcgtccgcacctatttcggctggcgcgaggtcttcgtgctcggcggagcggtgatcctctacgcgctgatcctcgggccgcgctggccctatgtcgccttctgggcggtgccgtcgctcatggcctcggtgcagctgttcgtcttcggcaccttcctgccgcaccgccttgaggccggcccgttcgccgaccgccacaacgcggcctcgacacggctcgccgtgcccttgtcgctgctgacctgctatcatttcggcggctatcaccacgagcaccacctgcatccgggcgtgccgtggtgggcgctgccccggacccggacggggccgcggcgatga

SEQ ID NO: 71 - protein, 237 aa, Paracoccus sphaerophysae
MTRLRRPTLTSLGIAVGVTAAWLALHVAAVWRLDALAHPGQAVLALAGLTWLSVGLFIIAHDAMHGALAPGYPWVNAAIGRVALTLYAGFSWRKLIVKHMAHHLYAGTASDPDFGHGHRPVVLWYLDFVRTYFGLREVVVLGGATIAYALILGPRWPYVAFWAIPSILASIQLFVFGTWLPHREAGAPFTDRHRARSTALSVPLSLLTCFHFGGFHHEHHLHPAVPWWALPKTRVAR

SEQ ID NO: 72 - DNA, 714 bp, Paracoccus sphaerophysae
atgacacgccttcgccgccccaccctgaccagcctcgggatcgccgtcggggtaaccgccgcttggctggcgttgcatgtcgccgccgtgtggcggctggacgcgctggcgcatccggggcaggccgtgctggcgcttgccgggctgacatggctgtcggtcgggctgttcatcatcgcccacgacgcgatgcacggcgcactggcgccgggctacccgtgggtcaacgccgcgatcgggcgggttgcgctgacgctttatgccgggttctcgtggcgcaagctgatcgtcaagcacatggcccaccacctctacgcggggaccgcgagcgatcccgacttcggccacggccaccggccggtcgtgctgtggtatctggacttcgtgcgcacctatttcggcttgcgcgaggtggtggtcctgggcggggcgaccattgcctatgcgctgatccttggcccgcgctggccctatgttgccttttgggcgatcccgtcgatcctcgcttcgatccagctgttcgtcttcggcacctggctgccgcaccgcgaggccggggcgccgttcaccgaccgccaccgcgcccgctccaccgcgctcagcgtgccgctgtcgctgctgacctgtttccacttcggtggatttcaccacgaacatcacctgcaccccgccgtgccgtggtgggcgctgcccaagacgcgggtcgcgcgatga

SEQ ID NO: 73 - protein, 239 aa, Sphingomonase sp. SRS2
MTNRAAQRAVGLTLAALVFGSWLLLQILALFLVQIDTLHPLLIMILVATLCWLDVGLFIIAHDAMHGSLAPGSPRLNRVIGTVMLAAYAGFSFDRLRPKHMAHHLTPGTVADPDFCERSPANPFRWYVAFFREYFGWREVLVLTLLVGVMTFAARVPYANLLLFWALPAILASVQLFLFGTFLPHAHRGPDFVDGHRARSSNFGWITSLLTCYHFGYHHEHHLHPHLPWWRLPSARCRG

SEQ ID NO: 74 - DNA, 720 bp, Sphingomonase sp. SRS2
atgactaatcgcgctgcgcaacgagccgttggcctcacgcttgccgcactggtgttcggcagctggcttctactccagatactcgctctcttcctcgtccagatcgacacgctgcacccgctgctgatcatgatactggtggcgacgctgtgctggctcgacgtcggcttgttcatcatcgcgcatgacgccatgcacgggtcactcgcaccgggatcgccgcgcctcaaccgggtgatcggcacggtgatgctcgcggcctatgcgggtttctcgttcgaccggcttcgcccaaagcatatggcgcaccacctgacgccggggaccgtggcggatcccgatttctgcgaacgatcgccagccaacccattccgctggtatgtcgctttcttccgcgaatatttcgggtggagggaagtcctcgtcctcaccctcctggtcggcgtgatgaccttcgccgcccgcgtgccctatgccaatctgctgcttttctgggcgctacccgcgatactcgcttcggtgcagctattcctgttcggcaccttcctgccgcacgcccaccgcggcccggacttcgtcgatgggcatcgcgcgcgtagctcgaacttcggttggatcacgtcgctgctgacatgctatcatttcggctatcatcacgaacatcaccttcatccgcatctgccctggtggcggctgccgagtgctcgatgccgggggtga

SEQ ID NO: 75 - protein, 422 aa, Porphyrobacter cryptus
MVDSPPNPAEAGTAQARHDVVIVGGGLAGGLIALALHRHAPACRFLLIEAGRTLGAHHRWSWFETDIPERERGLMAGFALSGWEEGYDITFPRLGRTLPTAYRSLASAEFHRALMAELPAERVMLAARAQALDAGGVTLGDGRRLAARCVLDCRPFQPSKALAGGWQVFLGQHIRCNTPHGLTRPVIMDASVAQLAPHGNGEAYRFVYVLPLSATEVFVEDTYYADQPRMDAGVLKGRIAAYARSNGWEGEVIDEEAGILPVISGGDFAAALAEVTIPGVALAGARGGFSHPLTSYTLPFAVSNALAIARLIAARPGLTGSELAAFCLARARRHWRAMAYYRMLSRMLFEAAEPGKRVVVFEHFYALRGALVERFYAGRSTWADRLRILTGKPPVAISRAIGALFSSGRPLETRLQTLETPA

SEQ ID NO: 76 - DNA, 1269 bp, Porphyrobacter cryptus
tcatgccggggtctccaatgtctgaagtctggtctcgagcggcctgcccgaggagaacagcgcgccgatcgcgcgcgagatagctacgggcggcttgccggtgaggatgcgcagccggtcagcccaggtcgagcggccggcatagaagcgctcgaccagcgcgccgcgcagggcatagaaatgctcgaacaccaccacccgcttgcccggctcggccgcctcgaacagcatccggctaagcatgcggtaataggccatggcgcgccagtggcgtcgggcgcgggcgaggcagaaggcggcaagttcactgccggtgagccccgggcgcgccgcgatcagccgggcgatggcgagcgcattgctgaccgcaaagggcagggtgtagctggtgagcgggtgcgaaaagccgccccgcgcgcccgccagcgccaccccggggatggtcacttctgcgagcgcagcggcaaaatccccgcccgagatcaccggcaggatgcccgcttcctcgtcgatcacctcgccctcccagccgttgctcctggcataggcggcgatccggcccttcaagacgccagcatccatccttggctggtcggcatagtaggtgtcctcgacgaagacctcggttgccgagagcggcaggacatagacgaagcgataggcttcgccattgccatggggcgccagctgcgcgacgctcgcatccatgatcaccgggcgggtgaggccgtggggcgtgttgcagcggatatgctggccgaggaacacctgccagccgccggcaagcgccttcgagggctggaacgggcggcaatcgagcacgcagcgcgcggcaaggcgcctgccatcgccgagcgtcaccccgccggcatcgagcgcctgtgcccgcgccgcgagcatcacccgctcggccggcagctcggccatcagcgcgcggtggaactcggcgctggcgagcgagcgataggcggtgggcagggtgcggccaagccgcgggaaggtgatgtcatagccctcctcccagccactgagggcaaagcccgccatcaggccgcgctcgcgctcgggaatatcggtctcgaaccagctccagcggtgatgggcgccgagcgtgcgcccggcctcgatcagcaggaagcggcacgcgggggcatggcggtggagcgcgagcgcaatcagcccgccggcaagcccgccgccgacgatgacgacatcatgccgcgcctgcgcggtgccggcctcggcagggttggggggtgaatcgaccat

SEQ ID NO: 77 - protein, 388 aa, Franconibacter pulveris
MKPQWDVILVGAGLANGLIAWRLHQRQPHLKVLLLEAGPQAGGNHTWSFHEQDLTPEQLAWLSPLVAHRWQGYDVRFPDRERTLPGAYLTITSDHFAHQLRQKLNAALRCNVSVMHVAPGEVTLDNGERLTARAVIDGRGPLLNEHIACGYQAFIGQQWRLSAPHGLERPILMDATVDQQRGYRFVYTLPLSPDTLLIEDTYYTNHPEINQAQARANIHAYAAAQGWRLESLQREERGCLPITLAGNPRAFWRSKAGQPCSGLRAGLFHATTGYSLPQSAQLADLLAWQTDFSHPSLYALIARHAQKQWRRQRFFRLLNRMLFLAGRPEQRWRVMARFYGLSDGIIARFYAGRLTPADMARLLSGKPPVPVGEALLAILKQPLRQRGR

SEQ ID NO: 78 - DNA, 1167 bp, Franconibacter pulveris
tcatcgtcctcgttgccgcagcggttgtttaagtatcgccagcagcgcctcgccgacgggcaccggcggcttgccgctgagaaggcgcgccatatcggcgggcgtaagccgcccggcgtagaagcgggcgataatcccgtcgctcaggccataaaagcgcgccatcacccgccagcgctgctcaggtcgcccggcgagaaacagcatgcggttaagcaggcgaaagaagcgttgccgccgccactgtttctgcgcgtggcgggcgataagcgcgtaaagcgaggggtgcgaaaaatcggtttgccaggcgagcaggtcggcaagctgcgccgactgcggcaatgaatagccggtggtggcgtgaaacagcccggcgcgcaggccgctgcagggctggcccgctttgctgcgccagaaagcgcgagggttgccggcaagcgtaatgggcaggcagccgcgctcttcccgttgcaggctctccagccgccagccctgggcggcggcataggcgtgaatattggcccgcgcctgcgcctgatttatctccgggtggttggtgtagtacgtgtcttcaatcaacagcgtgtcaggcgaaagcggcagggtatagacgaagcggtagccccgctgctggtcgaccgtggcgtccatcaaaatggggcgctcaaggccgtgaggtgctgagagccgccactgctggccgataaacgcctggtagccgcacgcgatatgctcattcagcagcgggccgcggccgtcgatgactgcgcgggcggtgagccgctcgccgttatccagcgtcacttcgccgggcgccacgtgcataaccgatacgttgcagcgcagcgccgcgttaagcttctggcggagctgatgggcgaaatggtcggaagtgatagtcaggtaggcgcccggcagcgtgcgctcacggtcaggaaagcgcacgtcgtaaccctgccagcggtgcgccaccagcggcgagagccaggcgagctgctccggcgtgaggtcctgctcgtggaaagaccaggtgtggttgccgcccgcctgcggcccagcttcgagcagcaagaccttgaggtgcggctggcgctggtgaagacgccaggcgataagcccgttcgccagccccgcgcccaccagaatcacatcccattgcggcttcat

SEQ ID NO: 79 - protein, 409 aa, Phormidium tenue
MQDVLVIGAGPAGLSLVAALAETGLAVQVLALGDPAHPWPNTYGIWVDELEDVGLVPFLEHRWKDCVVHVNSGPVPLHREYGLLSNDRLQAHWLNQAEQHQVTWCRDKAIHIEHRPTHTQVTTASGEKLTARLVVDATGHQTALVQRPAASDLSFQAAYGIVGRFSKPPTDPQQMVLMDFRDDYLTPAQRQEPPTFLYAMDLGDGVYFVEETSLAHHPAISIEVLTQRLHQRLRHRGIEVEYIHHVERCLFPMNQPLPDFTQRVVGFGGAASMVHPASGYMVGALLRRGPGLAGAIAAALSSPHTPPDQVAHQAWQALWPADRVRKHYLYLFGLENLMAFDAPQLHQFFNAFFNLPTDDWAGFLTDNLSLPEVVQAMLGLFGRAPNPVRWGLMRSVFSHGHLLGRTLMS

SEQ ID NO: 80 - DNA, 1230 bp, Phormidium tenue
atgcaggatgtgttggtcattggcgcggggccggcggggctgtcgctagtggcagccctggcagagacggggctggcggtgcaggtgttggccctgggcgatccggcgcacccctggcccaacacctacggcatctgggtcgatgaactcgaagatgtgggtctggtgccgtttttagagcatcgctggaaggattgcgtggtgcatgtcaacagtggccccgtgcccctgcatcgcgagtatgggttactgagcaacgatcgcctgcaagcccactggctaaaccaggctgagcagcaccaagtcacctggtgccgagataaagccattcacattgagcacaggcccacccatacccaggtcacgaccgcatcgggtgaaaagcttactgctcggctggtggtcgatgccacggggcatcagacggcgctagtgcagcgtccggctgcctcagacctgtcgtttcaagcggcctacggtattgtggggcggttttctaagccgcctaccgaccctcagcaaatggtgctgatggacttccgcgacgactatctcacacccgcccagcgacaggaaccgcccacctttctctacgcgatggatttgggcgatggggtgtactttgtggaagaaacctcgctggcccaccacccggccatttctatagaggtgctgacgcagcggctgcaccagcggctgcgccaccggggtattgaggtcgagtatattcaccatgtcgagcggtgcctgtttccgatgaaccagccgctgcccgattttacccagcgggtggtgggctttggtggggctgctagcatggtgcatccagcatcgggctatatggtgggggcactgctgcggcgggggccggggctggctggggcgatcgccgctgcccttagctcaccccacacgccccctgaccaagtggcccaccaggcttggcaagccctatggcccgccgatcgcgttcgcaagcactacctctatcttttcggcctcgaaaatctgatggcctttgacgcgccgcagctgcatcagttttttaatgccttttttaacctgcccaccgatgactgggccgggtttttgaccgacaatctctcgctgcctgaggtagtgcaggccatgctggggctgtttggccgcgcccctaatccggtgcgctgggggctgatgcgatcggtctttagccatggccacctgctggggcgcaccttgatgagctaa

SEQ ID NO: 81 - protein, 388 aa, Siccibacter colletis
MKTWDLILVGGGLANGLIAWRLKQRHPTLTILMLESGDAPGGNHTWSFHHGDLTPAQHSWLAPLVSHRWQGYDVHFPGLTRTLPHDYFTVTSERFAQAISALLQDHLLANVTVSRITDREVALSDGRRFTAGAVIDGRGYRPSPHLAIGYQSFVGQEWRLARPHGLERPILMDARVAQGAGYRFVYTLPLSADRVLIEDTHYIDGAQLDASVARQHIADYARDQGWQLSELVREEQGSLPITLAGDNTAFWQALRRQPVSGLRAGLFHATTGYSLPLAVRLADHIANAPSLHQAALFDLIARFAGHHWQTQGYFRLLNRMLFLAGAPDQRWRVMQRFYQLDEQLIARFYAGQLQATDKLRILMGKPPVPVVGALKALLHTHHPLRAKK

SEQ ID NO: 82 - DNA, 1167 bp, Siccibacter colletis
atgaaaacatgggatctgattctggtgggcggcgggctggcgaacgggctgattgcctggcggctgaagcagcgccatccgacgctgaccattctgatgcttgagagcggcgacgcgccgggtgggaatcatacctggtcctttcatcatggcgatcttaccccggcacagcatagctggctggcaccgctggtcagtcaccgctggcagggctacgatgtgcatttccccggcctgacgcgcaccctgccgcatgactatttcaccgttacctcggaacgctttgctcaggctatctccgccctgctgcaggatcaccttctggccaatgtgacggtgtcacgcataaccgatcgcgaagtggcgctgagcgatggacgccgctttaccgccggagcagtgatcgacgggcgtggctaccggccttcaccgcatctcgctattggttaccagtcgtttgtcggtcaggagtggcgtctggcccggccgcatggcctggagcgtccgatcctgatggatgcccgggtggctcaaggtgccgggtatcgcttcgtttataccctgcccctgagcgctgaccgggtgctgatcgaagacacccattatattgacggcgcgcagcttgacgccagcgtcgcccggcagcacatcgccgactatgcccgcgaccagggctggcagttaagcgaacttgtgcgtgaagaacagggttcactgccaattaccctcgctggcgacaacacagccttctggcaagcgctgcgccgccagccggtaagcgggctgcgcgccggactgtttcacgccactaccggctactccctgccgctggccgtcaggctggcggaccatatcgctaatgcgccctcgctgcatcaggcggcgctgttcgatctgattgcgcgcttcgccggtcaccactggcagacgcagggttatttccgtctgctcaaccgcatgctgtttctcgccggcgcaccggaccagcgctggcgcgtgatgcaacgtttttatcaacttgatgagcagctgattgcccgcttttatgccggccagctgcaggctaccgacaaactgcggatcctgatgggaaaaccgccggtgccggtggtcggggcgctgaaagccctgctgcacactcatcaccctttgcgagccaaaaaatga

SEQ ID NO: 83 - protein, 395 aa, Erythrobacter vulgaris
MSIEELDIAIVGGGLAGGTLAMALRKHAPDLSCALFEAGDVYGGNHRWSWFEHDLDEDGAALLEPFARTAWTGGNEVRFPGYSRRLESNYRSLDSRDFDAGIRRALPQEAIRTGARVVGLDGAGITLGTGERIAARAVVDCRDALPSEHLAGGWQVFLGQHLKTTAQHGIKRPVIMDATVNQPGAYRFVYLLPLGPDEIFVEDTYYADSPELDAEILRQRIADYAAGKDWQFEIVHEETGVLPVITDGDFEAYRASLSTPGVTLAGARGGFVHPLTSYTVPIAVRNALAIARAASDDIAALPEFVDSRAKQHWRDTAYYRLLGKMLFQAAEPAERYRVFERFYRLPEVLISRFYAARSTLPDKLRILTGKPPVSVSRAIRALLGKGAPLVQGKSP

SEQ ID NO: 84 - DNA, 1188 bp, Erythrobacter vulgaris
atgtccatcgaggagctagatatcgccatcgtcggcggcggccttgcgggcggcacgctggccatggccctgcgcaaacatgcacctgatctttcatgcgcgttgttcgaagcaggcgatgtttacggcggtaaccaccgctggagctggttcgagcatgatctcgacgaagacggtgccgccttgctcgaacctttcgccaggaccgcctggactggcgggaacgaggtccggtttcccggctattcacgcaggctggaaagcaattaccgttccctggacagccgcgacttcgatgccggaatacgcagggcgctaccgcaggaagccattcgcaccggcgcaagggtggtcgggctcgatggcgcaggcattacgcttgggaccggcgagcggattgcggcgcgcgccgtcgtcgattgccgcgatgccctgccgagcgagcatctcgccggcgggtggcaggttttcctgggccagcacctgaaaaccaccgcccagcatggcatcaagcggcctgtcatcatggatgcgactgtcaatcagccgggcgcataccgcttcgtctacctgttgccgctaggcccggacgagatctttgtcgaggatacctattacgccgacagtcccgagcttgacgcggagatcttgcgccagcgcatcgccgactatgcggccggaaaagactggcagttcgagattgtgcacgaagaaacgggcgtgctgccggtcatcaccgacggagatttcgaggcctaccgcgcttcgctcagcactccgggcgtcactctggcaggcgcgcgcggaggtttcgttcatccgctcacaagttacacagtgccgattgcggtccgcaacgcgctggcaatcgcaagggcggccagcgacgacatcgcggcgttgccggagtttgtcgacagccgcgccaaacagcactggcgcgacaccgcctattatcgtctcctcggcaagatgctgttccaggcggcagaacctgcggaacgatatcgcgtattcgagcgcttctatcgcctgcccgaagtgctcatttcccgtttttacgcagcccgctctaccttgcccgacaaattgcgcatcctcaccgggaaaccgcccgtatctgttagccgcgctataagagccctgcttggcaagggcgcaccgctggtccaaggaaagtccccatga

SEQ ID NO: 85 - protein, 395 aa, Rhizobium sp. Leaf321
MESGSHDLSLPIVLVGAGLASAVIAQRLSTVRDAPPIVILEATDTPFGEHTWSFHKADLQPSSLAWIAPLIAHEWDGQSVRFGSYERHLPSGYASLTSTSVSEAIERLPNVSLSKNTEVTALSAAQVTFANGSTQAASCVIDCRGYRPSASMVLGYQKFVGLEVELSEPHGLPHPVIMDATVDQLDGYRFVYLLPMSPTRVLIEDTRYSEGADLNDAVISEDIHTYATRQGWDITQVVRRENGVLPIVLAHDFERFWSELPADIPHAGLRAGLFHPTTGYSLPDAVHVADLIGENWPIGSAALPRAIRGHAARQHKKQGFYRLLNRMLFLAAEPDRRHLVLERFYKLPTPLIERFYAGRTTMTDILRILTGKPPVPIHRALACLSEAKLFENGSS

SEQ ID NO: 86 - DNA, 1188 bp, Rhizobium sp. Leaf321
atggagagcgggtcgcacgatctgtcgctgcccatcgtgctcgtcggggcaggtcttgccagcgcagtcatcgcgcaacggctctcgactgtccgtgatgcgccaccgatcgtcattctcgaagccacggacacgcccttcggcgagcatacctggtccttccacaaagcggatctgcaaccctccagccttgcctggatagcgcccctgatcgcccatgaatgggacggacagtcggtgcggttcggcagctatgagcgccacctgccgtccgggtatgcttcacttacatctacgtccgtcagcgaagccatcgaacgtctgccaaatgtgtcgctcagtaagaacaccgaggttactgcgctttcggctgcgcaggtcacgttcgccaatggcagtacgcaggccgcgtcatgtgtcatcgattgccgtggctatcgaccaagcgcgtcgatggtgctcggctaccagaagttcgtcggcctcgaagtggagctttcggagcctcacggcttgccgcatccggtcatcatggacgcaacggtcgaccagctcgacggctaccgattcgtctacctgcttccaatgtcgccgacgcgcgtgctgatcgaagacacacgctactcggaaggcgccgatctcaacgatgcggtgatatcagaggatatccacacctatgccacccgccagggttgggatatcacgcaggtcgtccgccgcgagaatggcgttctgccgatcgtgcttgcgcatgatttcgagcgcttctggtcagaattgccggcggatatcccccatgctggtctacgcgccggcctgttccatccgaccactggttacagcttgccggatgcggtccacgtcgctgatctgatcggcgagaactggcccatcggcagcgccgctctgccccgtgcgatccgcggacatgccgcccgccaacacaagaagcagggtttctaccgcctgctgaacagaatgctgttccttgccgcggagcctgatcggcgccatctcgtgctcgaacgtttctacaagctaccgacgccactgatcgaacggttttatgccggacggacaacgatgaccgatattctgcgcattcttaccggaaagcctccggtccccatccatagggcgctcgcctgtttgagcgaggccaagttgttcgagaacggatcctcatga

SEQ ID NO: 87 - protein, 390 aa, Parvularcula oceani
MADAYDLLFAGGGLSSGLTAYRVAQARSDLSIAIVEAAPRLGGNHTWSFHATDVAPGTLRWLTPFIVHDWPAQRVAFPRRERRIETPYRSVTSERLHEVVSAVPNISLLTGKPVKAVRRDGLTLEDGAVLSGPVLDGRGARSSPHLSLGFQKFLGQELRTARPHGVETPLIMDARVPQTDGYRFVYVLPLGPDILHVEDTYYADGDDLPREALREEIMAYAHRQGWEVAEILREEDGVLPIVLEGDIDGFWDAHRAGPAPLGLRAGLFHPVTGYSLPSAARLADEIAEACGSAIPAPGALFELVEDHARRQWEAQGFYRLLNRMLFRAAKPDLRYVVLQRFYGLPRALIERFYAGETTAADKARILAGKPPVPLGAALRSLRPQALLGQG

SEQ ID NO: 88 - DNA, 1173 bp, Parvularcula oceani
tcatccttgcccaagaagagcctgcggccgaagcgaccgcagcgccgcaccgagcggcactggcggcttgcctgccaatatgcgcgccttgtccgccgccgtcgtctctcctgcgtagaatcgttcgatcagcgcgcgaggcaagccatagaagcgttgaaggacgacgtaacgcaggtccggcttcgccgcgcggaacagcatgcggttgaggaggcggtagaacccctgggcttcccattgccgccgggcatggtcctcgacgagttcgaagagggcgccgggtgcgggaatcgcggacccgcaggcctccgcgatctcgtcggcgagccgcgccgcgctcggcaggctgtagccggtcacggggtggaagagcccggcccgcaggcccagcggcgcagggcctgcgcgatgggcatcccagaagccgtcgatgtcgccttcgagcacgatgggcaggacgccgtcctcctcgcgcaggatctcggcgacctcccagccctgccggtgcgcataggccatgatctcttcgcgcagcgcctcgcgcggcaggtcgtcgccatcggcataataggtgtcctcgacatgcaggatgtcggggccgagcggcaggacatagacgaagcggtagccgtcggtctggggcacccgcgcatccatgatgagcggcgtttcgaccccgtggggccgcgcggtgcgcaattcctgtccgaggaatttctggaagccgagcgagagatggggcgagcttcgcgcgccgcgcccgtcgaggaccgggcctgacaatacggcaccgtcttccagggtcaggccgtcgcgccgcacagccttcaccggcttcccggtcaggagcgagatgttcggaacggccgagacgacctcgtggagccgctcggaggtcacgctgcgatagggcgtttcgatccgccgctctcgcctcggaaaggcgacgcgctgggccggccagtcatggacgatgaagggggtgagccagcggagcgtgcccggcgccacgtcggtggcgtggaagctccaggtgtggttgccgccgaggcgcggcgccgcctccacgatggcaatggaaaggtctgaccgcgcctgcgccacccgataggcggtcaggccggaggagagccctccgcctgcaaaaagaagatcgtaagcgtctgccac

SEQ ID NO: 89 - protein, 188 aa, Franconibacter pulveris
MLVLWNTLIVITTFCLMEIVATLAHKYIMHGWGWGWHRSHHEPRHGWFEVNDLYAVVFALLAIVLIALGTAGWWPLQWLGAGMTLYGLFYFLVHDGLVHRRWPFNYIPRRGYLKRLYQAHRLHHAVNGKEGCVSFGFLYAAKPEALQAELRRRHGRKPKADAARARRDGKPVAQSESRAQDLPPKSAE

SEQ ID NO: 90 - DNA, 567 bp, Franconibacter pulveris
atgcttgtgctgtggaatacgcttatcgtcatcacgacattttgcctgatggagatagtggcgacgcttgcccataaatacatcatgcacggctggggatggggctggcaccgttcgcaccacgagccgcgccacggctggtttgaggtcaacgatctctacgcggtggtgttcgcgctgctcgccatcgtgctgatcgcgctgggaacagcgggctggtggccgctgcaatggcttggcgcaggcatgacgctctacgggctgttctatttcctcgtgcacgacgggctggtgcaccgccgctggccgtttaactacattccgcgccgcggctaccttaagcggctttaccaggcgcaccggctgcaccacgcggtgaatggcaaagaggggtgcgtctccttcggttttctctacgcggcgaagccggaagcgttgcaggcggagctgcgccgccgtcacgggcgcaaacctaaagcggacgccgccagagcccggcgggacgggaaacctgtcgcgcagagcgagagccgagcgcaagacctgccgccgaaatcagcagagtga

SEQ ID NO: 91 - protein, 185 aa, Siccibacter colletis
MLALWNTGIVILTIIIMEGVATFAHKYIMHGWGWGWHLSHHSPRKGKFELNDMYAVVFALLAIVLIYAGTEGYWPLQWIGAGMTGYGIIYFIVHDGLVHQRWPFRYIPRRGYLKRLYMAHRLHHAVRGREGCVSFGFIYAPPVEKLQAVLRARHGRPASAGAARDADRAAATPPSGKRSPASRRR

SEQ ID NO: 92 - DNA, 558 bp, Siccibacter colletis
atgcttgcgttgtggaacaccgggatcgttattctcactattattatcatggaaggcgtggcgacgtttgcccacaagtacatcatgcacggctggggatggggatggcatctgtcgcaccactcgccgcggaaagggaagtttgagcttaacgatatgtatgccgtggtgtttgcgctgctggctatcgtcctgatttatgccggcaccgaaggctactggccgctgcagtggatcggcgcgggcatgaccggatacggcatcatctactttatcgtccacgacggcctggtccaccagcgctggccgttccgctatattccgcgtcgcggctatctgaagcgtctgtatatggcgcaccggctgcatcacgcggtgcgcggacgggaagggtgcgtctcctttggctttatttacgcgcctccggtcgagaaactacaggcggtactgcgcgcccgccacggacgccccgctagcgcgggcgctgccagagatgcggatcgcgcggcggccactccgccttccgggaagcggtcgcctgcttcacgccggcgataa

SEQ ID NO: 93 - protein, 180 aa, Cronobacter malonaticus
MIVLYNVAIVLLTVAAMEVVAALTHKYVMHGWGWGWHLSHHSPRKGWFEVNDLYAVVFAGVAILLIALGAGGRWPLQWIGAGMTLYGALYFIVHDGLVHQRWPFRYVPRRGYLKRLYLAHRLHHAVRGREGCVSFGFLYAPPVAKLQAVLRERNGRPARAAAARAPKGETTTTRRENSQP

SEQ ID NO: 94 - DNA, 543 bp, Cronobacter malonaticus
atgatagtgctgtataacgtagcgattgtgctgctgacggtcgcggcgatggaagtcgtggcggcgctgacgcacaaatatgtaatgcatggctggggatggggctggcatctttcccaccattcgccgcgcaaaggctggtttgaagtcaacgatctctacgcggtggtgtttgcgggcgtggcgattttgcttattgcgcttggggcaggcggacgctggccgttgcagtggattggcgcgggcatgacgctttacggcgcgctttacttcatcgtgcatgacgggctggtccaccagcgctggccatttcgctatgtgccgcgccggggctatctgaagcggctctatctggcgcaccggctgcatcatgcggtgcgcgggcgcgaaggctgcgtctcctttggttttctctacgccccgccggtggctaagctgcaggcggtgctgcgtgagcgtaacggtcgtcctgcgcgcgcggccgctgccagagcgccgaagggcgagacgacgacgacgcgccgcgagaactcacagccataa

SEQ ID NO: 95 - protein, 511 aa, Deinococcus murrayi
MPDYDVLVMGAGHNALVTAAYAARAGLKVGVFERRHIVGGAVSTEELVPGYRFDYGGSAHILIRMTPVVRELELTRHGLHYLEVDPMFHASDGETPWFVWRDAERTARELEALFPGQGEAYQRFLNDWTPFARSVADLFNSAPGPLEMGKMVLGSGRGREWQTQLSRILRPYGDVAREYFSEERVRAPLTWMAAQSGPPPSDPLSAPFLLWHPLYHEGGVARPKGGSGGLTRALRRAIEDYGGEVHVNAPVRRILVRGGRAQGVELEGGERYTARAVVSGAHILTTAGALPDEHVPEAARRVRVGNGFGMILRLALSEKVRYRQHHEPESRVGLGLLIKDERQLAQAYGEYLAGEPTRDPPLIAMSFSAVDDSLAPPGGEVLWLWAQYYPYELASGSWETRTAEARENILRSFEHYAPGTRDTIVGELVQTPQWLETNLGLHRGNVMHLEMSFDQMFAFRPWMAASGYRWPGVGGLYLTGASTHPGGGIMGASGRNAARVLLRDLTRRGWK

SEQ ID NO: 96 - DNA, 1536 bp, Deinococcus murrayi
atgccagactacgacgtactcgtgatgggagcgggccacaacgccctggtgacggcggcctacgcggccagggccgggctgaaggtgggggtcttcgagcgccgccatatcgtcgggggggcggtgagcaccgaagaactggtgcccggctaccgcttcgactacgggggcagcgcccacatcctgattcggatgaccccggtggtgcgcgagctggagctgacccgccacggcctgcattacctcgaagtggaccccatgttccacgcctcggacggcgagacgccatggtttgtctggcgtgacgctgagcgcaccgcccgcgagctggaggccctctttcccggtcagggggaggcgtaccagcgcttcttgaacgactggaccccctttgcccgctcggtggccgacctctttaactcggcaccggggccgctggagatgggcaagatggtgctgggcagtggccggggccgcgagtggcagacccagctctcgcgcatcctgcggccttacggggacgtggcgcgggaatacttctcggaagagcgggtgcgggcaccgctgacctggatggcggcgcaaagcggcccgccgccgtcggaccccctgagtgcgccttttttgctgtggcaccccctctaccacgagggcggggtggcgcggcccaagggcggcagcggcggcctgacgcgggcgctgcggcgggcgatcgaggattacggcggcgaggtccacgtcaatgccccggtgcggcggattctggtgcgagggggcagggcgcagggcgtcgagctggaggggggcgagcgctacaccgcccgcgcggtcgtctccggtgcccacatcctgacgacggcgggggcgctgcccgacgagcacgtcccggaggcggcgcggcgggtgcgggtgggcaacggcttcggcatgattctgcggctggccctgagcgagaaggtgcgctaccgccagcaccacgagccggagtcgagggtggggctgggcctcctcatcaaggacgagcggcaactcgcgcaggcgtacggcgagtacctcgcgggtgagccgacccgtgacccgcccctcatcgccatgagcttttcggcggtcgacgacagccttgcgccccccggcggcgaggtgctgtggctgtgggcgcagtactacccctacgagctggcctccggcagttgggagacgcgcacggcggaagcgcgggagaacatcctgcgctcgttcgagcactacgcaccgggcacgcgggataccatcgtgggagaactcgtgcagacgccgcagtggttggagacgaacctcggcctgcaccggggcaacgtgatgcacctggagatgagctttgaccagatgttcgccttccggccctggatggccgcgagcgggtaccgctggccgggcgtggggggcctgtacctgacgggcgcgagcacccaccccggcggcggcatcatgggggcgtcggggcggaatgcggcgcgggtactgctgcgggacctgacccggcgggggtggaagtga

SEQ ID NO: 97 - protein, 505 aa, Deinococcus maricopensis
MGGGHNALVTAAYAARAGYRVGVFERRHVIGGAVSTEELVPGYRFDYGGSAHILIRMTPVVQELELARFGLQYLELDPLFHCSDGDTPWFIHRSADRTAHELETLFPGQGDAYARFMRDWTPFARAVNDLFLSAPSPLEMGKMMRRSGGELGMQAQLARILRPYGDVAREYFTEERVRAPLTWMAAQSGPPPTEPLSAPFLLWHPLYHEGGVARPRGGSGGLTRALARAIEAYGGQVHLNAAVSRILVERGRATGVELADGSRFTARAVVSGTHVLATAGALPPEHVPEAARNVRVGNGFGMVLRLALKEQVRYRVHHEPASRVGLGLLIRDERQLARAYGDYLAGEPTRDPPLVAMSFSAVDDSLAPPGGEVLWLWAQYYPFQLARGTWAQRELEAREGILNAFEHFAPGTRDNIVGELVQTPQWLHDHMGLVHGNVMHLEMGFDQMFAFRPFMAASQYRWPGVRDLYLTGASTHPGGGIMGASGRNAARALLHSLSRRGWRRA

SEQ ID NO: 98 - DNA, 1518 bp, Deinococcus maricopensis
atgggcggcgggcataacgccctggtgaccgccgcgtacgccgcgcgggccgggtaccgcgtgggcgtgttcgagcggcggcacgtgatcggcggcgccgtcagcaccgaggaactcgtgcccggctaccgcttcgactacggtggcagcgcgcacatcctgatccgcatgaccccggtcgtgcaggaactggagctcgcgcggttcgggctgcagtacctcgaactcgacccgctgttccactgcagcgacggcgacacaccgtggttcattcaccgcagcgccgaccgcaccgcgcacgaactggaaacgctgttccccgggcagggcgacgcgtacgcgcggttcatgcgcgactggacgccgttcgcgcgcgccgtgaacgatctgttcctgagcgcgcccagcccgctcgaaatgggcaagatgatgcgccgcagcggcggcgaactgggcatgcaggcgcaactggcgcgcatccttaggccgtacggggacgtcgcgcgcgagtacttcacggaggagcgcgtgcgcgcgccgctcacgtggatggcggcgcagtccgggccgcccccgaccgagccgctcagcgcgccgttcctgctgtggcacccgctgtaccacgagggcggcgtcgctcgcccccgcgggggctccggaggcctcacgcgcgccctggcgcgcgccatcgaggcgtacggtgggcaggtgcacctgaacgcggccgtgtcacggattctggtggagcggggccgcgcgacgggcgtggaactcgcggacgggtcgcgcttcacggcgcgcgccgtcgtgagcggcacgcacgtgctcgccaccgccggagcgctgccgccggagcacgtgccggaagcggcgcgcaacgtccgcgtgggcaacgggttcgggatggtcctgcggctcgccctgaaagagcaggtgcgctaccgcgtgcaccacgaacccgcgagccgcgtcggcctgggcctgctgatccgcgacgagcggcagctggcgcgcgcgtacggcgactacctcgcgggcgaacccacccgcgacccacccctcgtcgccatgagcttctcggcggtggacgacagcctcgctccgcccggcggggaggtgctgtggctgtgggcgcagtactacccgttccagctcgcgcgcggcacgtgggcgcagcgggagctggaggcgcgcgaaggcatcctgaacgccttcgagcatttcgcgccgggcacgcgcgacaacatcgtcggcgagctggtgcagacgccgcagtggctgcacgaccacatgggcctcgtgcacggcaacgtgatgcaccttgagatgggcttcgatcagatgttcgcgttccggccattcatggccgccagccagtaccgctggccgggcgtgcgcgacctgtacctgacgggcgcgagcacgcacccgggcggcggcatcatgggcgcgtccgggcggaacgccgcgcgggcgctgctgcacagcctgtccagacgcgggtggcgccgagcgtga